(19) United States
(12) Patent Application Publication
(10) Pub. No.: US 2006/0286611 A1
(43) Pub. Date: Dec. 21, 2006

(54) Antibodies against biotinylated histones and related proteins and assays related thereto

(75) Inventors: Janos Zempleni, Lincoln, NE (US); Gautam Sarath, Lincoln, NE (US)

Correspondence Address: SHERIDAN ROSS PC, 1560 BROADWAY, SUITE 1200, DENVER, CO, 80202, US

(73) Assignee: Board of Regents of University of Nebraska, Lincoln, NE (US)

(21) Appl. No.: 11/173,982

(22) Filed: Jun. 30, 2005

Related U.S. Application Data
(60) Provisional application No. 60/674,221, filed on Apr. 22, 2005.

(51) Int. Cl. G01N 33/574; G01N 33/53; C07K 16/40

(52) U.S. Cl. 435/7.23; 435/7.5; 530/388.26

(57) ABSTRACT

Described are specific biotinylation sites in histones, polypeptide fragments of histones comprising such biotinylation sites, and antibodies that selectively bind to such biotinylated sites. Also described are methods to detect biotinylation in a sample, to detect biotinyl transferase activity in a sample, to identify regulators of biotinylation, and to detect activities associated with histone biotinylation. Also described is an assay to detect or measure histone debiotinylation.

### CROSS-REFERENCE TO RELATED APPLICATIONS

This application claims the benefit of priority under 35 U.S.C. § 119(e) from U.S. Provisional Application No. 60/674,221, filed Apr. 22, 2005. The entire disclosure of U.S. Provisional Application No. 60/674,221 is incorporated herein by reference.

### GOVERNMENT SUPPORT

This invention was supported, in part, by federally funded Grant Nos. DK 60447, 1 P20 RR16469, DK 063945, each awarded by the National Institutes of Health, and by Grant No. EPS-0346476, awarded by the National Science Foundation. The government has certain rights to this invention.

### REFERENCE TO SEQUENCE LISTING

This application contains a Sequence Listing submitted on a compact disc, in duplicate. Each of the two compact discs, which are identical to each other pursuant to 37 CFR § 1.52(e)(4), contains the following file: “Sequence Listing”, having a size in bytes of 38 kb, recorded on 30 Jun. 2005. The information contained on the compact disc is hereby incorporated by reference in its entirety pursuant to 37 CFR § 1.77(b)(4).

### FIELD OF THE INVENTION

The present invention generally relates to the identification of biotinylation sites in histones; to polypeptide fragments of histones comprising such biotinylation sites; to antibodies that selectively bind to such sites, and to assays or methods for detecting biotinylation in a sample, for detecting biotinyl transferase activity in a sample, for identifying regulators of biotinylation, and for detecting activities associated with histone biotinylation, all such assays and methods using the biotinylation sites, peptides and antibodies of the invention. The present invention also relates to an assay for debiotinylation of a sample.

### BACKGROUND OF THE INVENTION

Histones are small proteins (11 to 22 kDa) that mediate the folding of DNA into chromatin. The following five major classes of histones have been identified in eukaryotic cells: H1, H2A, H2B, H3, and H4 (Wolffe 1998). DNA is wrapped around octamers of core histones, each consisting of one H3-H3-H4-H4 tetramer and two H2A-H2B dimers, to form the nucleosomal core particle. Histone H1 associates with the DNA connecting nucleosomal core particles. Nucleosomes are stabilized by electrostatic interactions between negatively charged phosphate groups in DNA and positively charged ε-amino groups (lysine residues) and guanidino groups (arginine residues) in histones.

Histones consist of a globular C-terminal domain and a flexible N-terminal tail (Wolffe 1998). The amino terminus of histones protrudes from the nucleosomal surface; lysine, arginine, serine, and glutamate residues in the amino terminus are targets for acetylation, methylation, phosphorylation, ubiquitination, poly (ADP-ribosylation), and sumoylation (Wolffe 1998, Fischle et al., 2003; Jenuwein and Allis, 2001; Boulikas et al., 1990; Shiio and Eisenman, 2003). These modifications play important roles in chromatin structure, regulating processes such as transcriptional activation or silencing of genes, DNA repair, and mitotic and meiotic condensation of chromatin. Some regions in C-terminal domains (e.g., hinge regions) are also exposed at the nucleosomal surface, and are potential targets for covalent modifications (Wolffe, 1998). For example, K120 in histone H2B is a target for ubiquitination (Fischle et al., 2003), and K108, K116, K120, and K125 in histone H2B are targets for acetylation (Zhang et al., 2003). Histone H2A is unique among core histones in having its C-terminal tail exposed at the nucleosomal surface (Wolffe, 1998; Luger et al., 1997). Consistent with this observation, the following modifications have been identified in the C-terminus of histone H2A and its variant H2A.X: ubiquitination of K119 (Fischle et al., 2003; Ausio et al., 2001) and phosphorylation of S139 (Downs et al., 2004; Paull et al., 2000), respectively.

Evidence has been provided for a novel modification of histones: covalent binding of the vitamin biotin (Hymes et al., 1995; Stanley et al., 2001). Two enzymes can independently catalyze biotinylation of histones: biotinidase (EC 3.5.1.12), using biocytin (biotin-e-lysine) as a substrate (Hymes et al., 1995) and holocarboxylase synthetase, using biotin and ATP as a substrate (Narang et al., 2004). Biotinylation of histones is likely to play a role in processes such as gene silencing (Peters et al., 2002), cell proliferation (Stanley et al., 2001; Narang et al., 2004), and DNA repair or apoptosis (Peters et al., 2002; Kothapalli and Zempleni, 2004). These observations have important implications for human health. For example, alterations in the biotinylation pattern of histones might be an early signaling event in response to DNA damage. Second, mutations of the genes encoding biotinidase (Swango et al., 1998; Wolf et al, 2002; Moslinger et al., 2003) and holocarboxylase synthetase (Yang et al., 2001) have been documented; some of these mutations are fairly common (Wolf and Heard, 1991; Wolf, 1991). Fibroblasts from individuals with mutated holocarboxylase synthetase are deficient in histone biotinylation (Narang et al., 2004). Likewise, in vitro studies provided evidence that mutated biotinidase is not capable of catalyzing biotinylation of histones (Hymes et al., 1995). Future study may unravel abnormal patterns of gene silencing (Peters et al., 2002), cell proliferation (Stanley et al., 2001; Narang et al., 2004), and DNA repair or apoptosis (Peters et al., 2002; Kothapalli and Zempleni, 2004) in individuals carrying mutations of genes coding for biotinidase and holocarboxylase synthetase.

Although all five major classes of histones appear to be biotinylated in human cells (Stanley et al., 2001), prior to the present invention, the amino-acid residues that are targets for biotinylation had not yet been identified. The different post-translational modifications of histones can influence each other in synergistic or antagonistic ways, thereby mediating gene regulation. For example, phosphorylation of S10 inhibits methylation of K9 in histone H3, but is coupled with K9 and/or K14 acetylation during mitogenic stimulation in mammalian cells (Jenuwein and Allis, 2001). Conversely, deacetylation of K14 in histone H3 facilitates subsequent methylation of K9, leading to transcriptional silencing. Ultimately, modifications of histones affect the access of enzymes such as RNA polymerases and DNA repair enzymes to DNA. Identification of biotinylation sites in histones is the first step in deciphering the cross-talk between biotinylation and other covalent modification of histones that regulate gene expression.

The gap in the understanding of histone biotinylation has created a significant obstacle for investigating roles of biotinylated histones in cell biology, based on the following lines of reasoning. As long as biotinylation sites remain unknown, no site-specific antibodies to biotinylated histones can be generated. Such antibodies are invaluable tools (i) to study the cross-talk among modifications of histones, e.g., biotinylation and acetylation of lysine residues; (ii) to investigate cellular distribution patterns of biotinylated histones by using immunocytochemistry; and (iii) to investigate roles for biotinylation of histones in the regulation of transcriptional activity of genes by using chromatin immunoprecipitation assays.

Moreover, mechanisms mediating debiotinylation of histones are poorly understood. Circumstantial evidence has been provided that biotinidase might catalyze both biotinylation and debiotinylation of histones (Ballard et al., 2002). Variables such as the microenvironment in chromatin, and posttranslational modifications and alternate splicing of biotinidase might determine whether biotinidase acts as biotinyl histone transferase or histone debiotinylase (Zempleni, 2005).

Therefore, there remains a need in the art for information regarding the biotinylation of histones, for tools to investigate, evaluate and manipulate such biotinylation, and for new assays to determine the mechanism of histone biotinylation and its role in gene expression, gene silencing, cell proliferation, and DNA repair or apoptosis.

### SUMMARY OF THE INVENTION

One embodiment of the present invention relates to an isolated antibody or antigen-binding fragment thereof that selectively binds to a biotinylated histone selected from biotinylated histone H2A, biotinylated histone H3, and biotinylated histone H4. Preferably, the antibody or antigen-binding fragment thereof does not bind to a non-biotinylated histone. In one aspect, the antibody is a monoclonal antibody. In another aspect, the antigen binding fragment is an Fab fragment. In another aspect, the antibody is a humanized antibody. In another aspect, the antibody is a bispecific antibody. In yet another aspect, the antibody is a monovalent antibody. The invention further includes compositions including any of the isolated antibodies or antigen binding fragments described herein, and a delivery vehicle comprising any of the isolated antibodies or antigen binding fragments described herein linked to an agent to be delivered.

In one aspect of this embodiment, the antibody or antigen binding fragment thereof selectively binds to biotinylated histone H4. Such an antibody or antigen binding fragment thereof can selectively bind to: (a) an epitope comprising the second lysine residue from the N-terminus in histone H4, wherein the second lysine residue is biotinylated; or (b) an epitope comprising the third lysine residue from the N-terminus in histone H4, wherein the third lysine residue is biotinylated. In another aspect, such an antibody or antigen binding fragment thereof can selectively bind to: (a) an epitope comprising the lysine at position 8 of SEQ ID NO:6, or the equivalent position thereto in a non-human histone H4 sequence, wherein the lysine residue is biotinylated; or (b) an epitope comprising the lysine at position 12 of SEQ ID NO:6, or the equivalent position thereto in a non-human histone H4 sequence, wherein the lysine residue is biotinylated. In one aspect, such an antibody or antigen binding fragment thereof selectively binds to an amino acid sequence selected from: SEQ ID NO:6, SEQ ID NO:7 and SEQ ID NO:10, wherein said amino acid sequence is biotinylated. Preferably, the antibody or antigen binding fragment thereof does not cross-react with histones H1, H2A, H2B and H3.

In another aspect of this embodiment, the antibody or antigen binding fragment thereof selectively binds to biotinylated histone H3. Such an antibody or antigen binding fragment thereof can selectively bind to: (a) an epitope comprising the first lysine residue from the N-terminus in histone H3, wherein the first lysine residue is biotinylated; (b) an epitope comprising the second lysine residue from the N-terminus in histone H3, wherein the second lysine residue is biotinylated; or (c) an epitope comprising the fourth lysine residue from the N-terminus in histone H3, wherein the fourth lysine residue is biotinylated. In another aspect, such an antibody or antigen binding fragment thereof can selectively bind to: (a) an epitope comprising the lysine at position 4 of SEQ ID NO:5, or the equivalent position thereto in a non-human histone H3 sequence, wherein the lysine residue is biotinylated; (b) an epitope comprising the lysine at position 9 of SEQ ID NO:5, or the equivalent position thereto in a non-human histone H3 sequence, wherein the lysine residue is biotinylated; or (c) an epitope comprising the lysine at position 18 of SEQ ID NO:5, or the equivalent position thereto in a non-human histone H3 sequence, wherein the lysine residue is biotinylated. In one aspect, the antibody or antigen binding fragment thereof selectively binds to an amino acid sequence selected from the group consisting of: SEQ ID NO:5, SEQ ID NO:30 and SEQ ID NO:32, wherein said amino acid sequence is biotinylated. Preferably, the antibody or antigen binding fragment thereof does not cross-react with histones H1, H2A, H2B and H4.

In yet another aspect of this embodiment, the antibody or antigen binding fragment thereof selectively binds to biotinylated histone H2A. Such an antibody or antigen binding fragment thereof can selectively bind to: (a) an epitope comprising the second lysine residue from the N-terminus in histone H2A, wherein the second lysine residue is biotinylated; (b) an epitope comprising the third lysine residue from the N-terminus in histone H2A, wherein the third lysine residue is biotinylated; (c) an epitope comprising the first lysine residue from the C-terminus in histone H2A, wherein the first lysine residue is biotinylated; (d) an epitope comprising the second lysine residue from the C-terminus in histone H2A, wherein the second lysine residue is biotinylated; or (e) an epitope comprising the third lysine residue from the C-terminus in histone H2A, wherein the third lysine residue is biotinylated. In another aspect, such an antibody or antigen binding fragment thereof can selectively bind to: (a) an epitope comprising the lysine at position 9 of SEQ ID NO:2, or the equivalent position thereto in a non-human histone H2A sequence, wherein the lysine residue is biotinylated; (b) an epitope comprising the lysine at position 13 of SEQ ID NO:2, or the equivalent position thereto in a non-human histone H2A sequence, wherein the lysine residue is biotinylated; (c) an epitope comprising the lysine at position 125 of SEQ ID NO:2, or the equivalent position thereto in a non-human histone H2A sequence, wherein the lysine residue is biotinylated; (d) an epitope comprising the lysine at position 127 of SEQ ID NO:2, or the equivalent position thereto in a non-human histone H2A sequence, wherein the lysine residue is biotinylated; or (e) an epitope comprising the lysine at position 129 of SEQ ID NO:2, or the equivalent position thereto in a non-human histone H2A sequence, wherein the lysine residue is biotinylated. In one aspect, the antibody or antigen binding fragment thereof selectively binds to an amino acid sequence selected from the group consisting of: SEQ ID NO.:2, SEQ ID NO:3, SEQ ID NO:48, SEQ ID NO:49 and SEQ ID NO:52, wherein said amino acid sequence is biotinylated. Preferably, the antibody or antigen binding fragment thereof does not cross-react with histones H1, H2B, H3, and H4.

Another embodiment of the present invention relates to a method to detect biotinylated histones in a biological sample. The method includes contacting a biological sample containing histones with any antibody or antigen-binding fragment thereof described herein, and detecting the amount of antibody or antigen-binding fragment thereof that binds to the biological sample. In one aspect, the biological sample is a eukaryotic cell sample or a nuclear extract thereof.

Yet another embodiment of the present invention relates to a method to detect DNA damage in a cell. The method includes contacting a nuclear extract from a cell or tissue to be evaluated with any antibody or antigen-binding fragment thereof described herein, and measuring the amount of antibody that binds to histones in the extract as compared to a control sample that does not have DNA damage.

Another embodiment of the present invention relates to a method to detect biotinyl transferase activity in a biological sample. The method includes the steps of: (a) contacting a biological sample with a histone or polypeptide fragment thereof, wherein the polypeptide fragment thereof comprises at least one biotinylation site in the histone, and wherein the histone or polypeptide fragment thereof is not biotinylated prior to contact with the biological sample; (b) incubating the biological sample and histone or polypeptide fragment thereof with biocytin or biotin and ATP; and (c) measuring the amount of histone or polypeptide fragment thereof that is biotinylated after step (b), wherein the amount of biotinylated histone or polypeptide fragment thereof is indicative of the amount of biotinyl transferase activity in the biological sample. In one aspect, the biological sample is a nuclear extract from a mammalian cell. In another aspect, the histone is selected from the group consisting of histone H1, histone H2A, histone H2B, histone H3 and histone H4. In another aspect, the polypeptide fragment thereof is an at least about 8 amino acid polypeptide fragment selected from: (a) a polypeptide fragment of human histone H4 (SEQ ID NO:6), comprising at least one lysine residue selected from the group consisting of: the lysine at position 8 and the lysine at position 12; (b) a polypeptide fragment of human histone H3 (SEQ ID NO:5), comprising at least one lysine residue selected from the group consisting of: the lysine at position 4, the lysine at position 9 and the lysine at position 18; (c) a polypeptide fragment of human histone H2A (SEQ ID NO:2) or H2A.X (SEQ ID NO:3), comprising at least one lysine residue selected from the group consisting of: the lysine at position 9 and the lysine at position 13; and (d) a polypeptide fragment of human histone H2A (SEQ ID NO:2), comprising at least one lysine residue selected from the group consisting of: the lysine at position 125, the lysine at position 127 and the lysine at position 129. In one aspect, step (c) comprises detecting the amount of biotinylated histones or polypeptide fragments thereof by contacting the histones or polypeptide fragments thereof with an antibody that selectively binds to the histone or polypeptide fragment when the histone or polypeptide fragment is biotinylated and not to non-biotinylated histone or polypeptide fragment thereof.

In one aspect of this embodiment, the histone or polypeptide fragment in step (a) are immobilized in an assay well, and step (c) comprises the steps of: (i) washing the assay well to remove the biological sample and biocytin; (ii) incubating the immobilized histone or polypeptide fragment with an antibody that selectively binds to the histone or polypeptide fragment when the histone or polypeptide fragment is biotinylated and not to non-biotinylated histone or polypeptide fragment thereof; and (iii) measuring the amount of antibody in (ii) that is bound to the biotinylated histone or polypeptide fragment thereof to indicate the amount of biotinyl transferase activity in the biological sample. In this aspect, step (iii) can include contacting the antibody with a labeled secondary antibody and detecting the amount of bound label.

In another aspect of this embodiment of the invention, step (c) can include the steps of: (i) separating the proteins and polypeptides after step (b) by gel electrophoresis; (ii) performing an immunoblot of the gel using an antibody that selectively binds to the histone or polypeptide fragment when the histone or polypeptide fragment is biotinylated and not to non-biotinylated histone or polypeptide fragment thereof; and (iii) measuring the amount of antibody in (ii) that is bound to the biotinylated histone or polypeptide fragment thereof to indicate the amount of biotinyl transferase activity in the biological sample.

Yet another embodiment of the present invention relates to an assay to detect debiotinylase activity in a biological sample. The method includes the steps of: (a) incubating a biological sample with a biotinylated histone or a biotinylated polypeptide fragment thereof; (b) contacting the biological sample and biotinylated histone or fragment thereof with an avidin-conjugated detectable label; and (c) measuring the amount of avidin-conjugated detectable label that is bound to the biotinylated histone or fragment thereof after incubation with the biological sample as compared to prior to the incubation step. An amount of reduction in the biotinylation of the histone or fragment thereof after the incubation step indicates the amount of debiotinylase activity in the biological sample.

Another embodiment of the present invention relates to a method to identify regulators of histone biotinylation. The method includes the steps of: (a) contacting a putative regulatory compound of histone biotinylation with a histone or a polypeptide fragment thereof, wherein the polypeptide fragment thereof comprises at least one biotinylation site in the histone, and wherein the histone or polypeptide fragment thereof is not biotinylated prior to contact with the biological sample; (b) contacting the histone or polypeptide fragment thereof with an enzyme selected from the group consisting of biotinidase and holocarboxylase synthetase, either after step (a) or at the same time as step (a); (c) contacting the histone or polypeptide fragment thereof with a substrate for the enzyme in (b), either after step (b) or at the same time as step (b); and (d) measuring the amount of histone or polypeptide fragment thereof that is biotinylated after step (c). A decrease in the amount of biotinylated histone or polypeptide fragment thereof in the presence of the putative regulatory compound as compared to in the absence of the putative regulatory compound indicates that the putative regulatory compound is an inhibitor of histone biotinylation. Alternatively, an increase in the amount of biotinylated histone or polypeptide fragment thereof in the presence of the putative regulatory compound as compared to in the absence of the putative regulatory compound indicates that the putative regulatory compound is an enhancer of histone biotinylation. In one aspect, step (c) includes detecting the amount of biotinylated histones or polypeptide fragments thereof by contacting the histones or polypeptide fragments thereof with an antibody that selectively binds to the histone or polypeptide fragment when the histone or polypeptide fragment is biotinylated and not to non-biotinylated histone or polypeptide fragment thereof. In one aspect, the histone is selected from histone H1, histone H2A, histone H2B, histone H3 and histone H4. In another aspect, the polypeptide fragment thereof is an at least about 8 amino acid polypeptide fragment selected from: (a) a polypeptide fragment of human histone H4 (SEQ ID NO:6), comprising at least one lysine residue selected from the group consisting of: the lysine at position 8 and the lysine at position 12; (b) a polypeptide fragment of human histone H3 (SEQ ID NO:5), comprising at least one lysine residue selected from the group consisting of: the lysine at position 4, the lysine at position 9 and the lysine at position 18; (c) a polypeptide fragment of human histone H2A (SEQ ID NO:2) or H2A.X (SEQ ID NO:3), comprising at least one lysine residue selected from the group consisting of: the lysine at position 9 and the lysine at position 13; and (d) a polypeptide fragment of human histone H2A (SEQ ID NO:2), comprising at least one lysine residue selected from the group consisting of: the lysine at position 125, the lysine at position 127 and the lysine at position 129.

### BRIEF DESCRIPTION OF THE FIGURES OF THE INVENTION

FIG. 1 is a digitized image showing that lysine-to-alanine substitutions in peptides affect their enzymatic biotinylation.

FIGS. 2A and 2B are digitized images showing that amino acid modifications affect the biotinylation of histone H4.

FIGS. 3A-3C are digitized images showing that nuclear extracts from Jurkat cells contain histone H4, biotinylated at lysine-12.

FIG. 4 is a digitized image showing the biotinylation of K4, K9 and K14 in the N-terminal tail in histone H3.

FIG. 5 is a digitized image showing the biotinylation of K18 and K23 in the N-terminal tail in histone H3.

FIG. 6 is a digitized image showing that nuclear extracts from Jurkat cells contain histone H3, biotinylated at K4, K9, and K18.

FIG. 7 is a digitized image showing that K9 in histone H2A is a good target for biotinylation by biotinidase.

FIG. 8 is a digitized image showing that substitution of K15 in histone H2A with alanine renders K13 a good target for biotinylation by biotinidase.

FIG. 9 is a digitized image showing that the N-terminus of H2A.X is a good substrate for biotinylation by biotinidase.

FIG. 10 is a digitized image showing that K9 and K13 in histone H2A.X are targets for biotinylation by biotinidase.

FIG. 11 is a digitized image showing that K125, K127, and K127 in the C-terminus of histone H2A are targets for biotinylation by biotinidase.

FIG. 12 is a digitized image showing that methylation and acetylation of amino acids in the N-terminus of histone H2A affect the subsequent biotinylation of adjacent lysine residues by biotinidase.

FIGS. 13A and 13B are digitized images showing Western blot analysis of biotinylated histones.

FIG. 14 is a graph showing the spectrophotometric quantitation of TMB oxidation.

FIG. 15 is a graph showing the temporal pattern and protein dependence of histone debiotinylation.

FIG. 16 is a graph showing that the debiotinylation of histone H1 by nuclear enzymes from NCI-H69 cells depended on the pH of the incubation buffer.

FIG. 17 is a graph showing that the activities of histone debiotinylases in nuclear extract from human cells depended on the tissue from which cells originated.

FIG. 18 is a graph showing the activities of histone debiotinylases at various phases of the cell cycle.

### DETAILED DESCRIPTION OF THE INVENTION

The present invention generally relates to the discovery by the present inventors of biotinlyation sites in histones, and particularly in histones H4, H3, and H2A, and to the provision of: (1) polypeptide fragments of histones comprising such biotinylation sites; (2) antibodies that selectively bind to such biotinylation sites in histones; (3) an assay for biotinyl transferase activity in biological samples; (4) an assay to quantify activities of histone debiotinylases in biological samples; (5) a method to identify regulators of histone biotinylation; and (6) a variety of methods of use of the antibodies and assays described herein to evaluate and modulate the effects of histone biotinylation on, for example, the regulation of gene expression, the regulation of cell proliferation, and the regulation of the cellular response to DNA damage. For example, the tools and assays of the invention can be used to evaluate the affect of DNA damage on: (i) the abundance of biotinylated histones, (ii) the activity of histone biotinyl transferase, and (iii) interactions between biotinylation and acetylation of histones; and will unravel many new interactions of the histone-code. In addition, the anti-biotinylated histone antibodies disclosed herein are provided in kits for quantifying histone biotinyl transferase activity, as well as analysis of biotinylated histones in biological samples.

Biotinylation sites in histones were unknown to researchers prior to the present invention. Thus, there is significant improvement of existing technology and the potential to greatly enhance detection of specific modifications in histones provided by the present invention. This invention provides research and diagnostic tools for a newly discovered modification of histones that is believed to play a role in gene expression (including gene silencing), cell proliferation and DNA repair or apoptosis. Prior to the present invention, technology for detecting biotinylation of histones relied on the use of avidin and avidin-related reagents. These reagents are non-specific and cannot be used to document histone biotinylation sites with any degree of precision.

As described in detail below, the present inventors have developed a peptide-based procedure to identify biotinylation sites in histones. Using this assay, the inventors have identified biotinylation sites in human histone H4 (see Example 1), human histone H3 (see Example 2), and human histone H2A (see Example 3), and have thus clearly described and demonstrated an assay that can now be used to identify the biotinylation sites in human histone 2B and H1. Specifically, the following biotinylation sites have been identified by the present inventors in human histones (described in detail herein): K9, K13, K125, K127, and K129 in histone H2A; K4, K9, and K18 in histone H3; and K8 and K12 in histone H4. In addition, the inventors have produced and characterized monoclonal and polyclonal antibodies that selectively recognize biotinylated sites in the various human histones, which are valuable tools for research and therapeutic applications, and can be used to trace and quantify biotinylated histones under various experimental and in vivo conditions. The antibodies of the present invention can be further used to study the “cross-talk” between different histone modifications, such as the interaction between acetylation and biotinylation of histones.

The present invention also relates to the use of the antibodies described herein, or antigen binding fragments thereof, or compounds that bind to the same epitope as the antibodies described herein, as tools in a variety of assays for the detection of enzyme activity, biotinylation activity and regulatory compound identification, as well as diagnostic tools to locate the site of biotinylated histones in a cell or tissue sample. Such reagents can be used, for example, to identify DNA damage in a cell or tissue and to localize the site of the DNA damage.

The present invention also relates to the use of the antibodies described herein, or antigen binding fragments thereof, or compounds that bind to the same epitope as the antibodies described herein, as targeting moieties to deliver compounds (e.g., drugs) to biotinylated histones in a cell or tissue. For example, such reagents could be used to target drugs to a site of DNA damage, or to modulate the expression of genes involved in DNA replication and repair, or to modulate chromatin structure. Inhibitors of the expression of genes or chromatin structure would be useful, for example, in cancer therapy.

The present invention also relates to methods to identify regulators of histone biotinylation, including regulators that enhance biotinylation and inhibit biotinylation. Such regulators can be used to manipulate a variety of cellular events modulated by histones including, but not limited to, gene expression (including gene silencing), cell proliferation and DNA repair or apoptosis. The method can include the identification of regulators of enzymes that mediate biotinylation of histones (biotinidase and holocarboxylase synthetase). Again, the identification herein of biotinylation sites in histones and antibodies that bind to such sites are valuable reagents for use in such assays.

The present inventors have also developed a novel assay to quantify the activities of histone debiotinylases in extracts from eukaryotic cells. Using this assay, the inventors have shown (i) that human cell nuclei contain histone debiotinylase activity; (ii) that debiotinylation of histones is mediated by debiotinylases rather than proteases; (iii) that the activities of histone debiotinylases are greater in cells derived from lung and lymphoid tissues compared with liver and placenta and enzyme activity in HCT-116 colon cancer cells was slightly less that the enzyme activities in NCI-H69; (iv) that debiotinylation of histones is mediated by biotinidase and, perhaps, other histone debiotinylases; (v) that biotinidase accumulates in the cell nucleus, consistent with the cellular distribution of histone debiotinylase activity; and (vi) that the activities of histone debiotinylases depend on the cell cycle: activities are maximal during S phase, and are minimal during G2 and M phase of the cycle. This assay can be used to further evaluate debiotinylase activity in eukaryotic cells. Furthermore, the identification of the biotinylation sites and antibodies described herein greatly enhances the specificity of this assay.

Polypeptides of the Invention

Accordingly, one embodiment of the invention relates to the identification of biotinylation sites on histones, and the use of such sites to provide various natural and synthetic polypeptide fragments of biotinylated histones for use in the methods of the invention. As discussed above, histones are small proteins that mediate the folding of DNA into chromatin. The following five major classes of histones have been identified in eukaryotic cells: H1, H2A, H2B, H3, and H4 (Wolffe 1998). DNA is wrapped around octamers of core histones, each consisting of one H3-H3-H4-H4 tetramer and two H2A-H2B dimers, to form the nucleosomal core particle. Histone H1 associates with the DNA connecting nucleosomal core particles. Nucleosomes are stabilized by electrostatic interactions between negatively charged phosphate groups in DNA and positively charged ε-amino groups (lysine residues) and guanidino groups (arginine residues) in histones.

Histone H1 associates with the DNA connecting the nucleosomal core particles and functions in the compaction of chromatin into higher order structures. Histone H1 may be involved in early apoptotic events through polyADP-ribosylation of the histone. The nucleotide and amino acid sequences of human histone H1 are known. For example, the amino acid sequence for human histone H1 (isoform 1) can be found in GenBank Accession No. NP_{—}005316, and is represented herein by SEQ ID NO:1.

Histone H2A and H2A.X contain biotinylation motifs in their N- and C-terminal domains (present inventors, data not shown). The N- and C-terminal regions of histone H2A have important functions in telomeric silencing in yeast (Wyatt et al., 2003). Phosphorylation of histone H2A.X plays a role in the cellular response to DNA damage (Paull et al., 2000). Various posttranslational modifications are known to occur in histone H2A, e.g., phosphorylation of S1 (Pantazis and Bonner, 1981), acetylation of K5, K9 (Goll and Bestor, 2003) and K13 (Zhang et al., 2003), ubiquitination of K119 (Fischle et al., 2003; Ausio et al., 2001), phosphorylation T120 (Aihara et al., 2004), and methylation of K125 or K127 (Zhang et al., 2003). Likely, these modifications affect subsequent biotinylation (Example 1). Collectively, identification of biotinylation sites in histones H2A and H2A.X is likely to produce valuable insights into roles of these histones in chromatin structure and genomic stability. The nucleotide and amino acid sequences of human histone H2A and H2A.X are known. For example, the amino acid sequence for human histone H2A.1 can be found in GenBank accession number M60752, and is represented herein by SEQ ID NO:2, and the amino acid sequence for human histone H2A.X can be found in GenBank accession number P16104 and is represented herein by SEQ ID NO:3.

Histone H2B plays a role in the cellular response to DNA damage and perhaps cell death and has an important function in the phosphorylation of S14 (Cheung et al., 2003). The nucleotide and amino acid sequences of human histone H2B are known. For example, the amino acid sequence for human histone H2B (member A) can be found in GenBank Accession No. NP_{—}003509, and is represented herein by SEQ ID NO:4.

Histone H3 has a pivotal role in regulating gene expression. The nucleotide and amino acid sequences of human histone H3 are known. For example, the amino acid sequence for human histone H3 can be found in GenBank Accession No. NP_{—}066403, and is represented herein by SEQ ID NO:5.

Histone H4 plays a central role in organizing the DNA-histone complex and in regulating the transcriptional activity of genes (Wolffe, 1998; Fischle et al., 2003). Post-translational modifications of H4 appear to be essential for cell cycle progression. The nucleotide and amino acid sequences of human histone H4 are known. For example, the amino acid sequence for human histone H4 can be found in GenBank Accession No. NM_{—}175054, represented herein by SEQ ID NO:6. The amino-acid sequence of H4 is highly conserved among species.

An isolated protein, according to the present invention, is a protein (including a polypeptide or peptide) that has been removed from its natural milieu (i.e., that has been subject to human manipulation) and can include purified proteins, partially purified proteins, recombinantly produced proteins, and synthetically produced proteins, for example. As such, “isolated” does not reflect the extent to which the protein has been purified. An isolated protein useful according to the present invention can be isolated from its natural source, produced recombinantly or produced synthetically. Smaller peptides (polypeptides) useful in the present invention (e.g., in assays or methods of the invention, as regulatory peptides or for antibody production) are typically produced synthetically by methods well known to those of skill in the art.

As used herein, the term “homologue” is used to refer to a protein or peptide which differs from a naturally occurring protein or peptide (i.e., the “prototype” or “wild-type” protein) by minor modifications to the naturally occurring protein or peptide, but which maintains the basic protein and side chain structure of the naturally occurring form. Such changes include, but are not limited to: changes in one or a few amino acid side chains; changes one or a few amino acids, including deletions (e.g., a truncated version of the protein or peptide) insertions and/or substitutions; changes in stereochemistry of one or a few atoms; and/or minor derivatizations, including but not limited to: methylation, glycosylation, phosphorylation, acetylation, myristoylation, prenylation, palmitation, amidation and/or addition of glycosylphosphatidyl inositol. A homologue can have either enhanced, decreased, or substantially similar properties as compared to the naturally occurring protein or peptide. A homologue can include an agonist of a protein or an antagonist of a protein. A functional homologue is a homologue of a reference protein that may have any degree of structural similarity to the reference protein and has the same or essentially the same function as the reference protein. Typically, a functional homologue is structurally similar to the reference protein at least at conserved regions of the protein that are required for the function of the protein (e.g., catalytic domain, substrate binding site, cofactor binding site, DNA binding site, receptor or ligand binding site, signal transduction domains). An ortholog is an example of a functional homologue. Therefore, reference to a homologue can include an ortholog. An ortholog is encoded by a gene in two or more species that has evolved from a common ancestor and therefore has a common function.

According to the present invention, the minimum size of a protein, portion of a protein (e.g. a fragment, portion, domain, etc.), or region or epitope of a protein, is a size sufficient to serve as an epitope or conserved binding surface for the generation of an antibody or as a target in an in vitro assay. In one embodiment, a protein of the present invention is at least about 4, 5, 6, 7 or 8 amino acids in length (e.g., suitable for an antibody epitope or as a detectable peptide in an assay), or at least about 10 amino acids in length, or at least about 15 amino acids in length, or at least about 20 amino acids in length, or at least about 25 amino acids in length, or at least about 50 amino acids in length, or at least about 100 amino acids in length, or at least about 150 amino acids in length, and so on, in any length between 4 amino acids and up to the full length of a protein (e.g., a histone or an enzyme) or portion thereof or longer, in whole integers (e.g., 4, 5, 6, 7, 8, 9, 10, . . . 25, 26, . . . 500, 501, . . . ). Preferably, a polypeptide fragment of a histone useful in the present invention includes at least one biotinylation site in the histone from which the polypeptide fragment is derived or produced.

A polypeptide fragment of a histone useful in the present invention includes any polypeptide (e.g., a polypeptide of the minimum size as discussed above) that includes at least one biotinylation site as described herein. Useful polypeptides can include both biotinylated and non-biotinylated polypeptides. The fragment is not a full-length histone protein, and is most preferably between about 8 and about 100 amino acids in length, or between about 8 and about 75 amino acids in length, or between about 8 and about 50 amino acids in length, or between about 8 and about 40 amino acids in length, or between about 8 and about 30 amino acids in length, or between about 8 and about 20 amino acids in length, or is less than 20 amino acids in length. A polypeptide fragment of a histone can include any histone from any eukaryotic species, and preferably, from a mammalian species, and most preferably, from humans. Polypeptide fragments of histones containing a biotinylation site from histones H1, H2A, H2B, H3 and H4 are encompassed by the invention, and such fragments are exemplified herein for H4, H3 and H2A (see Examples 1, 2 and 3, respectively). Also described herein is a novel method for identifying the biotinylation sites in histones using synthetic peptides as substrates for biotinidase as set forth in detail in the Examples section.

Particularly useful polypeptides described herein include, but are not limited to, polypeptide fragments of at least 8 amino acids in length selected from: (a) fragments of histone H4, including a polypeptide comprising the second lysine residue from the N-terminus in histone H4, a polypeptide comprising the third lysine residue from the N-terminus in histone H4, or a polypeptide comprising both of the lysine residues; (b) fragments of histone H3, including a polypeptide comprising the first lysine residue from the N-terminus in histone H3, a polypeptide comprising the second lysine residue from the N-terminus in histone H3, a polypeptide comprising the fourth lysine residue from the N-terminus in histone H3, or a polypeptide comprising two or all three of these residues; (c) fragments of histone H2A or H2A.X, including a polypeptide comprising the second lysine residue from the N-terminus in histone H2A or H2A.X, a polypeptide comprising the third lysine residue from the N-terminus in histone H2A or H2A.X, a polypeptide comprising the first lysine residue from the C-terminus in histone H2A, a polypeptide comprising the second lysine residue from the C-terminus in histone H2A, a polypeptide comprising the third lysine residue from the C-terminus in histone H2A, or a polypeptide comprising both N-terminal residues or two or all three C-terminal residues.

With particular regard to histone H4, preferred polypeptide fragments also include: a polypeptide comprising the lysine at position 8 of SEQ ID NO:6, or the equivalent position thereto in a non-human histone H4 sequence, a polypeptide comprising the lysine at position 12 of SEQ ID NO:6, or the equivalent position thereto in a non-human histone H4 sequence, or a polypeptide comprising both biotinylation sites. Some preferred H4 polypeptides include SEQ ID NO:7 and SEQ ID NO:10, although many others are described in Example 1 and are encompassed by the invention.

With particular regard to histone H3, preferred polypeptide fragments include: a polypeptide comprising the lysine at position 4 of SEQ ID NO:5, or the equivalent position thereto in a non-human histone H3 sequence, a polypeptide comprising the lysine at position 9 of SEQ ID NO:5, or the equivalent position thereto in a non-human histone H3 sequence, a polypeptide comprising the lysine at position 18 of SEQ ID NO:5, or the equivalent position thereto in a non-human histone H3 sequence, or a polypeptide comprising two or all three of the biotinylation sites. Some preferred H3 polypeptides include SEQ ID NO:30 and SEQ ID NO:32, although many others are described in Example 2 and are encompassed by the invention.

With particular regard to histone H2A (or histone H2A.X), preferred polypeptide fragments include: a polypeptide comprising the lysine at position 9 of SEQ ID NO:2, or the equivalent position thereto in a non-human histone H2A sequence, a polypeptide comprising the lysine at position 13 of SEQ ID NO:2, or the equivalent position thereto in a non-human histone H2A sequence, a polypeptide comprising the lysine at position 125 of SEQ ID NO:2, or the equivalent position thereto in a non-human histone H2A sequence, a polypeptide comprising the lysine at position 127 of SEQ ID NO:2, or the equivalent position thereto in a non-human histone H2A sequence, a polypeptide comprising the lysine at position 129 of SEQ ID NO:2, or the equivalent position thereto in a non-human histone H2A sequence, or a polypeptide comprising both of the N-terminal biotinylation sites or two or all three of the C-terminal biotinylation sites. Some preferred H2A or H2A.X polypeptides include SEQ ID NO:48, SEQ ID NO:49 and SEQ ID NO:52, although many others are described in Example 3 and are encompassed by the invention.

In one embodiment of the present invention, any amino acid sequence described herein can be produced with from at least one, and up to about 20, additional heterologous amino acids flanking each of the C- and/or N-terminal ends of the specified amino acid sequence. The resulting protein or polypeptide can be referred to as “consisting essentially of” the specified amino acid sequence. According to the present invention, the heterologous amino acids are a sequence of amino acids that are not naturally found (i.e., not found in nature, in vivo) flanking the specified amino acid sequence, or that are not related to the function of the specified amino acid sequence, or that would not be encoded by the nucleotides that flank the naturally occurring nucleic acid sequence encoding the specified amino acid sequence as it occurs in the gene, if such nucleotides in the naturally occurring sequence were translated using standard codon usage for the organism from which the given amino acid sequence is derived.

Antibodies and Antigen-Binding Fragments

Another embodiment of the invention relates to an antibody or an antigen binding fragment thereof that selectively binds to a biotinylated histone or a biotinylated polypeptide fragment thereof comprising a biotinylation site, wherein the antibody does not selectively bind to the non-biotinylated form of the histone or fragment thereof. Similarly, an antigen binding polypeptide with the same specificity is also particularly preferred for use in the present invention. In one aspect, the antibody selectively binds to the histone or fragment thereof in a manner such that the histone or fragment is inhibited or prevented from binding to another antibody or another protein or DNA with which it may normally (under natural or physiological conditions) interact. Particularly preferred antibodies and antigen binding fragments thereof include any of the antibodies specifically described herein, and can include antibodies that selectively bind to the biotinylated forms of any of the histones or polypeptide fragments thereof described above or in the Examples.

Antibodies (and antigen binding fragments thereof) that selectively bind to biotinylated histones and biotinylated polypeptide fragments thereof according to the invention are described and exemplified in detail herein. In one embodiment, the antibody or antigen binding fragment thereof binds to a conserved binding surface or epitope of such a protein (e.g., a biotinylated histone) or fragment thereof that is conserved among animal species, and particularly mammalian, species (i.e., the antibody is cross-reactive with a biotinylated histone or fragment thereof from two or more different mammalian species). In another embodiment, the antibody or antigen binding fragment thereof binds to a conserved binding surface or epitope of a particular histone (e.g., histone H4), but does not substantially bind to (does not cross-react with, or at most, only weakly cross-reacts with) other histones (e.g., histone H3). In another embodiment, the antibody or antigen-binding fragment thereof selectively binds to a conserved binding surface or epitope comprising a particular biotinylation site on the histone, but does not substantially bind to (does not cross-react with or at most only weakly cross-reacts with) a polypeptide or epitope comprising a different biotinylation site on the same histone.

Based on the identification of biotinylation sites in at least three histones as described in the Examples, the present inventors have produced and characterized several antibodies that bind to biotinylated histones and biotinylated fragments thereof. Such antibodies are described in detail in the Examples. Preferred antibodies or antigen-binding fragments thereof of the invention include antibodies or fragments that selectively bind to a biotinylated histone selected from biotinylated histone H2A, biotinylated histone H3 and biotinylated histone H4. The antibody or antigen-binding fragment thereof is further characterized in that it does not substantially bind to or cross-react with non-biotinylated histone.

With regard to biotinylated histone H4, the antibody or antigen binding fragment thereof preferably selectively binds to an epitope selected from: (a) an epitope comprising the second lysine residue from the N-terminus in histone H4, wherein the second lysine residue is biotinylated; (b) an epitope comprising the third lysine residue from the N-terminus in histone H4, wherein the third lysine residue is biotinylated; or (c) an epitope comprising both of these biotinylation sites, where one or both of the sites is biotinylated. Particularly preferred epitopes include: (a) an epitope comprising the lysine at position 8 of SEQ ID NO:6, or the equivalent position thereto in a non-human histone H4 sequence, wherein the lysine residue is biotinylated; (b) an epitope comprising the lysine at position 12 of SEQ ID NO:6, or the equivalent position thereto in a non-human histone H4 sequence, wherein the lysine residue is biotinylated; or (c) an epitope comprising both of these biotinylation sites, where one or both of the sites is biotinylated. One of skill in the art can readily align the sequence of a human histone (e.g., human histone H4) with the sequence of the equivalent histone from another animal species and determine the positions of the lysine residues that are biotinylated according to the present invention as described herein. For example, two specific sequences can be aligned to one another using BLAST 2 sequence as described in Tatusova and Madden, (1999), “Blast 2 sequences—a new tool for comparing protein and nucleotide sequences”, FEMS Microbiol Lett. 174:247-250, incorporated herein by reference in its entirety. Particular polypeptides against which antibodies of the invention can be raised and against which the antibodies of the invention bind are described in Example 1 and antibodies or antigen-binding fragments that selectively bind to such polypeptides are encompassed by the invention. In one embodiment, the antibody or antigen binding fragment thereof does not cross-react with histones H1, H2A, H2B and/or H3.

With regard to biotinylated histone H3, the antibody or antigen binding fragment thereof preferably selectively binds to an epitope selected from: (a) an epitope comprising the first lysine residue from the N-terminus in histone H3, wherein the first lysine residue is biotinylated; (b) an epitope comprising the second lysine residue from the N-terminus in histone H3, wherein the second lysine residue is biotinylated; (c) an epitope comprising the fourth lysine residue from the N-terminus in histone H3, wherein the fourth lysine residue is biotinylated; or (d) an epitope comprising two or three of these biotinylation sites. Particularly preferred epitopes include: (a) an epitope comprising the lysine at position 4 of SEQ ID NO:5, or the equivalent position thereto in a non-human histone H3 sequence, wherein the lysine residue is biotinylated; (b) an epitope comprising the lysine at position 9 of SEQ ID NO:5, or the equivalent position thereto in a non-human histone H3 sequence, wherein the lysine residue is biotinylated; (c) an epitope comprising the lysine at position 18 of SEQ ID NO:5, or the equivalent position thereto in a non-human histone H3 sequence, wherein the lysine residue is biotinylated; or (d) an epitope comprising two or all three of these biotinylation sites. Particular polypeptides against which antibodies of the invention can be raised and against which the antibodies of the invention bind are described in Example 2 and antibodies or antigen-binding fragments that selectively bind to such polypeptides are encompassed by the invention. In one embodiment, the antibody or antigen binding fragment thereof does not cross-react with histones H1, H2A, H2B and/or H4.

With regard to biotinylated histone H2A, the antibody or antigen binding fragment thereof preferably selectively binds to an epitope selected from: (a) an epitope comprising the second lysine residue from the N-terminus in histone H2A, wherein the second lysine residue is biotinylated; (b) an epitope comprising the third lysine residue from the N-terminus in histone H2A, wherein the third lysine residue is biotinylated; (c) an epitope comprising the first lysine residue from the C-terminus in histone H2A, wherein the first lysine residue is biotinylated; (d) an epitope comprising the second lysine residue from the C-terminus in histone H2A, wherein the second lysine residue is biotinylated; (e) an epitope comprising the third lysine residue from the C-terminus in histone H2A, wherein the third lysine residue is biotinylated; or (f) an epitope comprising both N-terminal biotinylation sites or two or all three C-terminal biotinylation sites. Particularly preferred epitopes include: (a) an epitope comprising the lysine at position 9 of SEQ ID NO:2, or the equivalent position thereto in a non-human histone H2A sequence, wherein the lysine residue is biotinylated; (b) an epitope comprising the lysine at position 13 of SEQ ID NO:2, or the equivalent position thereto in a non-human histone H2A sequence, wherein the lysine residue is biotinylated; (c) an epitope comprising the lysine at position 125 of SEQ ID NO:2, or the equivalent position thereto in a non-human histone H2A sequence, wherein the lysine residue is biotinylated; (d) an epitope comprising the lysine at position 127 of SEQ ID NO:2, or the equivalent position thereto in a non-human histone H2A sequence, wherein the lysine residue is biotinylated; (e) an epitope comprising the lysine at position 129 of SEQ ID NO:2, or the equivalent position thereto in a non-human histone H2A sequence, wherein the lysine residue is biotinylated; or (f) an epitope comprising both N-terminal biotinylation sites or two or all three C-terminal biotinylation sites. Particular polypeptides against which antibodies of the invention can be raised and against which the antibodies of the invention bind are described in Example 3 and antibodies or antigen-binding fragments that selectively bind to such polypeptides are encompassed by the invention. In one embodiment, the antibody or antigen binding fragment thereof does not cross-react with histones H1, H2B, H3, and/or H4.

In one embodiment, the epitope recognized by an antibody of the invention can also be defined more particularly as being a linear or non-linear epitope located within the three-dimensional structure of a portion of a biotinylated histone, wherein the epitope contains at least one biotinylation site on the histone. As used herein, the “three dimensional structure” or “tertiary structure” of a protein refers to the arrangement of the components of the protein in three dimensions. Such term is well known to those of skill in the art. As used herein, the term “model” refers to a representation in a tangible medium of the three dimensional structure of a protein, polypeptide or peptide. For example, a model can be a representation of the three dimensional structure in an electronic file, on a computer screen, on a piece of paper (i.e., on a two dimensional medium), and/or as a ball-and-stick figure.

According to the present invention, an “epitope” of a given protein or peptide or other molecule is generally defined, with regard to antibodies, as a part of or site on a larger molecule to which an antibody or antigen-binding fragment thereof will bind, and against which an antibody will be produced. The term epitope can be used interchangeably with the term “antigenic determinant”, “antibody binding site”, or “conserved binding surface” of a given protein or antigen. More specifically, an epitope can be defined by both the amino acid residues involved in antibody binding and also by their conformation in three dimensional space (e.g., a conformational epitope or the conserved binding surface). An epitope can be included in peptides as small as about 4-6 amino acid residues, or can be included in larger segments of a protein, and need not be comprised of contiguous amino acid residues when referring to a three dimensional structure of an epitope, particularly with regard to an antibody-binding epitope. Antibody-binding epitopes are frequently conformational epitopes rather than a sequential epitope (i.e., linear epitope), or in other words, an epitope defined by amino acid residues arrayed in three dimensions on the surface of a protein or polypeptide to which an antibody binds. As mentioned above, the conformational epitope is not comprised of a contiguous sequence of amino acid residues, but instead, the residues are perhaps widely separated in the primary protein sequence, and are brought together to form a binding surface by the way the protein folds in its native conformation in three dimensions.

One of skill in the art can identify and/or assemble conformational epitopes and/or sequential epitopes using known techniques, including mutational analysis (e.g., site-directed mutagenesis); protection from proteolytic degradation (protein footprinting); mimotope analysis using, e.g., synthetic peptides and pepscan, BIACORE or ELISA; antibody competition mapping; combinatorial peptide library screening; matrix-assisted laser desorption/ionization time-of-flight (MALDI-TOF) mass spectrometry; or three-dimensional modeling (e.g., using any suitable software program, including, but not limited to, MOLSCRIPT 2.0 (Avatar Software AB, Heleneborgsgatan 21C, SE-11731 Stockholm, Sweden), the graphical display program O (Jones et. al., Acta Crystallography, vol. A47, p. 110, 1991), the graphical display program GRASP, or the graphical display program INSIGHT). For example, one can use molecular replacement or other techniques and the known three-dimensional structure of a related protein to model the three-dimensional structure of a histone and predict the conformational epitope of antibody binding to this structure, particularly given the identification of biotinylation sites in the histones provided by the present invention. Indeed, one can use one or any combination of such techniques to define the antibody binding epitope. The present invention provides a novel approach to identify biotinylation sites in histones (see Examples 1, 2 and 3), and the use of peptides comprising such sites to develop a variety of antibodies that selectively bind to such sites and to identify an epitope bound by such antibodies.

As used herein, the term “selectively binds to” refers to the specific binding of one protein to another (e.g., an antibody, fragment thereof, or binding partner to an antigen), wherein the level of binding, as measured by any standard assay (e.g., an immunoassay), is statistically significantly higher than the background control for the assay. For example, when performing an immunoassay, controls typically include a reaction well/tube that contain antibody or antigen binding fragment alone (i.e., in the absence of antigen), wherein an amount of reactivity (e.g., non-specific binding to the well) by the antibody or antigen binding fragment thereof in the absence of the antigen is considered to be background. Binding can be measured using a variety of methods standard in the art, including, but not limited to: Western blot, immunoblot, enzyme-linked immunosorbant assay (ELISA), radioimmunoassay (RIA), immunoprecipitation, surface plasmon resonance, chemiluminescence, fluorescent polarization, phosphorescence, immunohistochemical analysis, matrix-assisted laser desorption/ionization time-of-flight (MALDI-TOF) mass spectrometry, microcytometry, microarray, microscopy, fluorescence activated cell sorting (FACS), and flow cytometry.

One can also readily determine whether a given antibody “cross-reacts” with a protein other than the protein against which the antibody was produced (a cross-reacting protein) using such an assay. As used herein, an antibody is cross-reactive with a protein other than the protein against which the antibody was produced if the level of binding to the protein (the cross-reacting protein) is statistically significantly higher than the background control for the assay, such that the binding to the protein is indicated to be other than a non-specific binding. The level of binding of the cross-reactive antibody to the cross-reacting protein can be less than the level of binding of the antibody to the protein against which it was produced. A weakly cross-reacting antibody can be defined herein as an antibody that cross-reacts with a protein other than the protein against which it was produced at a level that is about 20% or less than the level of binding of the antibody to the protein against which it was produced. However, one of skill in the art will be able to determine an appropriate standard or limit for determining cross-reactivity based on the assay conditions and antibodies and standards or controls used.

One embodiment of the present invention includes an antibody or antigen binding fragment thereof that is a competitive inhibitor of the binding of the biotinylated histone or fragments thereof to the anti-biotinylated histone antibodies described herein. According to the present invention, a competitive inhibitor of biotinylated histone binding to an anti-biotinylated histone antibody of the present invention is an inhibitor (e.g., another antibody or antigen binding fragment or polypeptide) that binds to the biotinylated histone (or biotinylated fragment thereof) at the same or similar epitope as the known anti-biotinylated histone antibody of the present invention such that binding of the known anti-biotinylated histone antibody to the biotinylated histone is inhibited. A competitive inhibitor may bind to the target (e.g., a biotinylated histone) with a greater affinity for the target than the anti-biotinylated histone antibody. A competitive inhibitor can be used in a manner similar to that described herein for the anti-biotinylated histone antibodies of the invention. For example, one embodiment of the invention relates to an isolated antibody or antigen binding fragment thereof that specifically binds to a biotinylated histone, wherein the antibody or fragment thereof competitively inhibits an anti-biotinylated histone antibody as described herein for specific binding to the biotinylated histone or to the specific biotinylated fragment thereof. Another embodiment relates to an isolated antibody or fragment thereof that specifically binds to a biotinylated histone, wherein the isolated antibody or fragment thereof competitively inhibits a second antibody or fragment thereof for specific binding to the biotinylated histone, and wherein the second antibody or fragment thereof binds to an epitope of a histone comprising a biotinylation site that is biotinylated.

Competition assays can be performed using standard techniques in the art (e.g., competitive ELISA or other binding assays). For example, competitive inhibitors can be detected and quantitated by their ability to inhibit the binding of a biotinylated histone to a known, labeled anti-biotinylated histone antibody (e.g., such as those described in the Examples).

According to the present invention, antibodies are characterized in that they comprise immunoglobulin domains and as such, they are members of the immunoglobulin superfamily of proteins. Generally speaking, an antibody molecule comprises two types of chains. One type of chain is referred to as the heavy or H chain and the other is referred to as the light or L chain. The two chains are present in an equimolar ratio, with each antibody molecule typically having two H chains and two L chains. The two H chains are linked together by disulfide bonds and each H chain is linked to an L chain by a disulfide bond. There are only two types of L chains referred to as lambda (λ) and kappa (κ) chains. In contrast, there are five major H chain classes referred to as isotypes. The five classes include immunoglobulin M (IgM or μ), immunoglobulin D (IgD or δ), immunoglobulin G (IgG or λ), immunoglobulin A (IgA or α), and immunoglobulin E (IgE or ε). The distinctive characteristics between such isotypes are defined by the constant domain of the immunoglobulin and are discussed in detail below. Human immunoglobulin molecules comprise nine isotypes, IgM, IgD, IgE, four subclasses of IgG including IgG1 (γ1), IgG2 (γ2), IgG3 (γ3) and IgG4 (γ4), and two subclasses of IgA including IgA1 (α1) and IgA2 (α2).

Each H or L chain of an immunoglobulin molecule comprises two regions referred to as L chain variable domains (V_{L} domains) and L chain constant domains (C_{L} domains), and H chain variable domains (V_{H} domains) and H chain constant domains (C_{H} domains). A complete C_{H} domain comprises three sub-domains (CH1, CH2, CH3) and a hinge region. Together, one H chain and one L chain can form an arm of an immunoglobulin molecule having an immunoglobulin variable region. A complete immunoglobulin molecule comprises two associated (e.g., di-sulfide linked) arms. Thus, each arm of a whole immunoglobulin comprises a V_{H+L} region, and a C_{H+L} region. As used herein, the term “variable region” or “V region” refers to a V_{H+L} region (also known as an Fv fragment), a V_{L} region or a V_{H} region. Also as used herein, the term “constant region” or “C region” refers to a C_{H+L} region, a C_{L} region or a C_{H} region.

Limited digestion of an immunoglobulin with a protease may produce two fragments. An antigen binding fragment is referred to as an Fab, an Fab′, or an F(ab′)₂ fragment. A fragment lacking the ability to bind to antigen is referred to as an Fc fragment. An Fab fragment comprises one arm of an immunoglobulin molecule containing a L chain (V_{L}+C_{L} domains) paired with the V_{H} region and a portion of the C_{H} region (CH1 domain). An Fab′ fragment corresponds to an Fab fragment with part of the hinge region attached to the CH1 domain. An F(ab′)₂ fragment corresponds to two Fab′ fragments that are normally covalently linked to each other through a di-sulfide bond, typically in the hinge regions.

The C_{H} domain defines the isotype of an immunoglobulin and confers different functional characteristics depending upon the isotype. For example, μ constant regions enable the formation of pentameric aggregates of IgM molecules and a constant regions enable the formation of dimers.

The antigen specificity of an immunoglobulin molecule is conferred by the amino acid sequence of a variable, or V, region. As such, V regions of different immunoglobulin molecules can vary significantly depending upon their antigen specificity. Certain portions of a V region are more conserved than others and are referred to as framework regions (FW regions). In contrast, certain portions of a V region are highly variable and are designated hypervariable regions. When the V_{L} and V_{H} domains pair in an immunoglobulin molecule, the hypervariable regions from each domain associate and create hypervariable loops that form the antigen binding sites. Thus, the hypervariable loops determine the specificity of an immunoglobulin and are termed complementarity-determining regions (CDRs) because their surfaces are complementary to antigens.

Further variability of V regions is conferred by combinatorial variability of gene segments that encode an immunoglobulin V region. Immunoglobulin genes comprise multiple germline gene segments which somatically rearrange to form a rearranged immunoglobulin gene that encodes an immunoglobulin molecule. V_{L} regions are encoded by a L chain V gene segment and J gene segment (joining segment). V_{H} regions are encoded by a H chain V gene segment, D gene segment (diversity segment) and J gene segment (joining segment).

Both a L chain and H chain V gene segment contain three regions of substantial amino acid sequence variability. Such regions are referred to as L chain CDR1, CDR2 and CDR3, and H chain CDR1, CDR2 and CDR3, respectively. The length of an L chain CDR1 can vary substantially between different V_{L} regions. For example, the length of CDR1 can vary from about 7 amino acids to about 17 amino acids. In contrast, the lengths of L chain CDR2 and CDR3 typically do not vary between different V_{L} regions. The length of a H chain CDR3 can vary substantially between different V_{H} regions. For example, the length of CDR3 can vary from about 1 amino acid to about 20 amino acids. Each H and L chain CDR region is flanked by FW regions.

Other functional aspects of an immunoglobulin molecule include the valency of an immunoglobulin molecule, the affinity of an immunoglobulin molecule, and the avidity of an immunoglobulin molecule. As used herein, affinity refers to the strength with which an immunoglobulin molecule binds to an antigen at a single site on an immunoglobulin molecule (i.e., a monovalent Fab fragment binding to a monovalent antigen). Affinity differs from avidity which refers to the sum total of the strength with which an immunoglobulin binds to an antigen. Immunoglobulin binding affinity can be measured using techniques standard in the art, such as competitive binding techniques, equilibrium dialysis or BIAcore methods. As use herein, valency refers to the number of different antigen binding sites per immunoglobulin molecule (i.e., the number of antigen binding sites per antibody molecule of antigen binding fragment). For example, a monovalent immunoglobulin molecule can only bind to one antigen at one time, whereas a bivalent immunoglobulin molecule can bind to two or more antigens at one time, and so forth. Both monovalent and bivalent antibodies that selectively bind to histones are encompassed herein.

In one embodiment, the antibody is a bi- or multi-specific antibody. A bi-specific (or multi-specific) antibody is capable of binding two (or more) antigens, as with a divalent (or multivalent) antibody, but in this case, the antigens are different antigens (i.e., the antibody exhibits dual or greater specificity). For example, an antibody that selectively binds to a biotinylated histone according to the present invention can be constructed as a bi-specific antibody, wherein the second antigen binding specificity is for a desired target. Therefore, one bi-specific antibody encompassed by the present invention includes an antibody having: (a) a first portion (e.g., a first antigen binding portion) which binds to a biotinylated histone; and (b) a second portion which binds to another protein, such as a protein associated with a particular cell type or another intracellular protein. In this manner, the biotinylated histone antibody can be effectively targeted to a particular cell or tissue type and/or to a particular compartment in a cellular extract.

In one embodiment, antibodies of the present invention include humanized antibodies. Humanized antibodies are molecules having an antigen binding site derived from an immunoglobulin from a non-human species, the remaining immunoglobulin-derived parts of the molecule being derived from a human immunoglobulin. The antigen binding site may comprise either complete variable regions fused onto human constant domains or only the complementarity determining regions (CDRs) grafted onto appropriate human framework regions in the variable domains. Humanized antibodies can be produced, for example, by modeling the antibody variable domains, and producing the antibodies using genetic engineering techniques, such as CDR grafting (described below). A description various techniques for the production of humanized antibodies is found, for example, in Morrison et al. (1984) Proc. Natl. Acad. Sci. USA 81:6851-55; Whittle et al. (1987) Prot. Eng. 1:499-505; Co et al. (1990) J. Immunol. 148:1149-1154; Co et al. (1992) Proc. Natl. Acad Sci. USA 88:2869-2873; Carter et al. (1992) Proc. Natl. Acad. Sci. 89:4285-4289; Routledge et al. (1991) Eur. J. Immunol. 21:2717-2725 and PCT Patent Publication Nos. WO 91/09967; WO 91/09968 and WO 92/113831.

Isolated antibodies of the present invention can include serum containing such antibodies, or antibodies that have been purified to varying degrees. Whole antibodies of the present invention can be polyclonal or monoclonal. Alternatively, functional equivalents of whole antibodies, such as antigen binding fragments in which one or more antibody domains are truncated or absent (e.g., Fv, Fab, Fab′, or F(ab)₂ fragments), as well as genetically-engineered antibodies or antigen binding fragments thereof, including single chain antibodies, humanized antibodies (discussed above), antibodies that can bind to more than one epitope (e.g., bi-specific antibodies), or antibodies that can bind to one or more different antigens (e.g., bi- or multi-specific antibodies), may also be employed in the invention.

Genetically engineered antibodies of the invention include those produced by standard recombinant DNA techniques involving the manipulation and re-expression of DNA encoding antibody variable and/or constant regions. Particular examples include, chimeric antibodies, where the V_{H} and/or V_{L} domains of the antibody come from a different source as compared to the remainder of the antibody, and CDR grafted antibodies (and antigen binding fragments thereof), in which at least one CDR sequence and optionally at least one variable region framework amino acid is (are) derived from one source and the remaining portions of the variable and the constant regions (as appropriate) are derived from a different source. Construction of chimeric and CDR-grafted antibodies are described, for example, in European Patent Applications: EP-A 0194276, EP-A 0239400, EP-A 0451216 and EP-A 0460617.

Generally, in the production of an antibody, a suitable experimental animal, such as, for example, but not limited to, a rabbit, a sheep, a hamster, a guinea pig, a mouse, a rat, or a chicken, is exposed to an antigen against which an antibody is desired. Typically, an animal is immunized with an effective amount of antigen that is injected into the animal. An effective amount of antigen refers to an amount needed to induce antibody production by the animal. The animal's immune system is then allowed to respond over a pre-determined period of time. The immunization process can be repeated until the immune system is found to be producing antibodies to the antigen. In order to obtain polyclonal antibodies specific for the antigen, serum is collected from the animal that contains the desired antibodies (or in the case of a chicken, antibody can be collected from the eggs). Such serum is useful as a reagent. Polyclonal antibodies can be further purified from the serum (or eggs) by, for example, treating the serum with ammonium sulfate.

Monoclonal antibodies may be produced according to the methodology of Kohler and Milstein (Nature 256:495-497, 1975). For example, B lymphocytes are recovered from the spleen (or any suitable tissue) of an immunized animal and then fused with myeloma cells to obtain a population of hybridoma cells capable of continual growth in suitable culture medium. Hybridomas producing the desired antibody are selected by testing the ability of the antibody produced by the hybridoma to bind to the desired antigen.

A preferred method to produce antibodies of the present invention includes (a) administering to an animal an effective amount of a protein or peptide (e.g., biotinylated histone or peptide a biotinylation site thereof) to produce the antibodies and (b) recovering the antibodies. In another method, antibodies of the present invention are produced recombinantly. For example, once a cell line, for example a hybridoma, expressing an antibody according to the invention has been obtained, it is possible to clone therefrom the cDNA and to identify the variable region genes encoding the desired antibody, including the sequences encoding the CDRs. From here, antibodies and antigen binding fragments according to the invention may be obtained by preparing one or more replicable expression vectors containing at least the DNA sequence encoding the variable domain of the antibody heavy or light chain and optionally other DNA sequences encoding remaining portions of the heavy and/or light chains as desired, and transforming/transfecting an appropriate host cell, in which production of the antibody will occur. Suitable expression hosts include bacteria, (for example, an E. coli strain), fungi, (in particular yeasts, e.g. members of the genera Pichia, Saccharomyces, or Kluyveromyces,) and mammalian cell lines, e.g. a non-producing myeloma cell line, such as a mouse NSO line, or CHO cells. In order to obtain efficient transcription and translation, the DNA sequence in each vector should include appropriate regulatory sequences, particularly a promoter and leader sequence operably linked to the variable domain sequence. Particular methods for producing antibodies in this way are generally well known and routinely used. For example, basic molecular biology procedures are described by Maniatis et al. (Molecular Cloning, Cold Spring Harbor Laboratory, New York, 1989); DNA sequencing can be performed as described in Sanger et al. (PNAS 74, 5463, (1977)) and the Amersham International plc sequencing handbook; and site directed mutagenesis can be carried out according to the method of Kramer et al. (Nucl. Acids Res. 12, 9441, (1984)) and the Anglian Biotechnology Ltd. handbook. Additionally, there are numerous publications, including patent specifications, detailing techniques suitable for the preparation of antibodies by manipulation of DNA, creation of expression vectors and transformation of appropriate cells, for example as reviewed by Mountain A and Adair, J R in Biotechnology and Genetic Engineering Reviews (ed. Tombs, M P, 10, Chapter 1, 1992, Intercept, Andover, UK) and in the aforementioned European Patent Applications.

Alternative methods, employing, for example, phage display technology (see for example U.S. Pat. No. 5,969,108, U.S. Pat. No. 5,565,332, U.S. Pat. No. 5,871,907, U.S. Pat. No. 5,858,657) or the selected lymphocyte antibody method of U.S. Pat. No. 5,627,052 may also be used for the production of antibodies and/or antigen fragments of the invention, as will be readily apparent to the skilled individual.

Another aspect of the present invention generally relates to compositions comprising the biotinylated histone polypeptides and/or antibodies of the invention and methods of using such peptides, antibodies, or compositions. Compositions will be discussed in more detail below.

Yet another aspect of the invention relates to a delivery vehicle comprising any of the isolated antibodies or antigen binding fragments thereof described herein linked to an agent to be delivered. The antibodies and antigen-binding fragments of the invention can be linked by any suitable method, (e.g., covalently or non-covalently, including by recombinant means or by chemical means) to a drug or other compound that is to be targeted to a site of histone biotinylation. For example, one may wish to target a drug to a site of DNA damage in a cell by using an antibody or antigen binding-fragment thereof of the present invention. In this scenario, the delivery vehicle would be first be delivered intracellularly. In another embodiment, one may wish to deliver a reagent to a biotinylated histone for a diagnostic or research purpose, and the delivery vehicle of the invention can be used for such purpose.

Methods of the Invention

The invention also includes the use of the polypeptides and antibodies described herein in a variety of methods related to the biotinylation of histones. One embodiment of the invention is a method to detect biotinylated histones in a biological sample. The method includes contacting a biological sample containing histones with an antibody or antigen-binding fragment thereof of the present invention, and detecting the amount of antibody or antigen-binding fragment thereof that binds to the biological sample. For example, suitable biological samples can include, but are not limited to, a eukaryotic cell sample or a nuclear extract thereof. The method of contacting can be any suitable method of contacting or exposing the antibody or fragment thereof to the cell or extract thereof, such as by mixing, combining or plating, and can include steps of first treating the sample to make the histones in the sample accessible to the antibodies (e.g., by lysing, preparing nuclear extracts, etc.). Assay formats suitable for detecting the amount of antibody or antigen-binding fragment thereof that binds to the biological sample include, but are not limited to, Western blot, immunoblot, enzyme-linked immunosorbant assay (ELISA), in situ hybridization, radioimmunoassay (RIA), immunoprecipitation, microscopy, fluorescence activated cell sorting (FACS), and flow cytometry. All of such methods are well known in the art.

One extension of this embodiment of the invention includes a method to detect DNA damage in a cell, comprising contacting a nuclear extract from a cell or tissue to be evaluated with an antibody or antigen-binding fragment thereof according to the present invention, and measuring the amount of antibody that binds to histones in the extract as compared to a control sample that does not have DNA damage. The polypeptides and antibodies of the invention will be useful to evaluate or diagnose a variety of cellular mechanisms that are regulated or affected by biotinylation of histones, as described elsewhere herein. All such uses are encompassed by the invention.

Another embodiment of the invention relates to a method to detect biotinyl transferase activity in a biological sample. The method includes the general steps of: (a) contacting a biological sample with a histone or polypeptide fragment thereof, wherein the polypeptide fragment thereof comprises at least one biotinylation site in the histone, and wherein the histone or polypeptide fragment thereof is not biotinylated prior to contact with the biological sample; (b) incubating the biological sample and histone or polypeptide fragment thereof with a substrate for a biotinyl transferase (e.g., biocytin, a substrate for biotinidase or biotin and ATP, substrate for holocarboxylase synthetase); and (c) measuring the amount of histone or polypeptide fragment thereof that is biotinylated after step (b), wherein the amount of biotinylated histone or polypeptide fragment thereof is indicative of the amount of biotinyl transferase activity in the biological sample.

In this method, the biological sample can include any suitable sample where biotinyl transferase activity might be detected. Such a sample can include any eukaryotic cell or tissue sample, and preferably any mammalian cell or tissue sample, such as a nuclear extract from a mammalian cell, by way of example. The step of contacting can be achieved by any suitable method of contacting or exposing the antibody or fragment thereof to the biological sample, and will depend on the assay format used (microtiter plate, well of a larger plate, other substrate), and can include, but is not limited to, adding one component to another, mixing, combining or plating. The biological sample and the histone or fragment thereof can be contacted on a solid substrate or suspended in a liquid medium or buffer. The conditions under which the step of contacting occurs accounts for the number of cells or amount of extract or other sample per container contacted, the concentration of various components, and the incubation time. Determination of effective protocols can be accomplished by those skilled in the art based on variables such as the size of the container, the volume of liquid in the container, conditions known to be suitable for the particular biological sample and for the histones or polypeptides.

Histones or polypeptide fragments thereof may, in one embodiment, be immobilized on a substrate. Such a substrate can include any suitable substrate for immobilization of a protein or peptide, including any solid support, such as any solid organic, biopolymer or inorganic support that can form a bond with the protein or peptide without significantly affecting the activity and/or ability of the assay to detect the reaction in the assay. Exemplary organic solid supports include polymers such as polystyrene, nylon, phenol-formaldehyde resins, and acrylic copolymers (e.g., polyacrylamide). Exemplary biopolymer supports include cellulose, polydextrans (e.g., Sephadex®), agarose, collagen and chitin. Exemplary inorganic supports include glass beads (porous and nonporous), stainless steel, metal oxides (e.g., porous ceramics such as ZrO₂, TiO₂, Al₂O₃, and NiO) and sand.

For example, in one embodiment, 96-well plates are coated with polypeptide fragments of various histones; these fragments contain the following biotinylation sites that are described herein. For controls, peptides in which biotinylation sites have been deleted (e.g., lysine-8 in histone H4 has been replaced with an alanine residue) or in which biotinylation sites have been modified (e.g., acetylation of lysine-8) can be used. As a positive control, wells are coated with a peptide fragment that has been biotinylated chemically. The biological sample is then added to the wells containing the peptides bound therein. In another embodiment, the same peptides are simply mixed, for example in a buffer, with the biological samples. The histone or polypeptide fragment used in step (a) can include any of the histones or polypeptide fragments thereof described herein, wherein the fragments comprise at least one of the biotinylation sites in histones as exemplified by the present inventors.

The next step of the method includes incubating the mixture of biological sample and histones or polypeptides thereof with a biotin-providing substrate for the biotinylation of histones. Such a substrate can include, but is not limited to, biocytin (substrate for biotinidase) or biotin and ATP (substrate for holocarboxylase synthetase). The substrate can be added to the plate before, after, or at the same time as the biological sample, and is preferably added at the same time or after the addition of the biological sample. The biotinyl transferases (e.g., biotinidase or holocarboxylase synthetase) in biological samples will utilize the biocytin to conduct an in vitro biotinylation of the histone or fragments in the assay. The amount of biotinylated histone generated on the plate parallels the activity of histone biotinyl transferase in biological samples.

The period of incubation of the biological sample and the peptide being tested can be varied, but is at least long enough to allow the biotinylation of histones by any biotinyl transferases in the biological sample, and can be determined by one of skill in the art. Suitable incubation times are described in the Examples section. The timing for contact and incubation can also vary depending on the substrate used, the concentrations of components in the assay, and similar variables.

The step of measuring the biotinylation of the histones or fragments as a read-out for the assay can be performed by any suitable method, including an ELISA or Western blot. In one aspect, this step comprises detecting the amount of biotinylated histones or polypeptide fragments thereof by contacting the histones or polypeptide fragments thereof with an antibody or antigen binding fragment thereof of the present invention (i.e., an antibody or antigen-binding fragment thereof that selectively binds to the histone or polypeptide fragment when the histone or polypeptide fragment is biotinylated and not to non-biotinylated histone or polypeptide fragment thereof). For example, the histone or polypeptide fragments in step (a) can be immobilized in an assay well, and after the incubation with substrate, the method can include steps of (i) washing the assay well to remove the biological sample and biocytin; (ii) incubating the immobilized histone or polypeptide fragment with the antibody; and (iii) measuring the amount of antibody in (ii) that is bound to the biotinylated histone or polypeptide fragment thereof to indicate the amount of biotinyl transferase activity in the biological sample. One can detect the antibody of the invention, for example, by using a secondary antibody incubation step, wherein the secondary antibody is labeled with a detectable label. In general, detectable labels include any label detectable by spectroscopic, photochemical, biochemical, immunochemical, electrical, optical or chemical means. Useful labels in the present invention include fluorescent dyes (e.g., fluorescein, texas red, rhodamine, green fluorescent protein, and the like), radiolabels (e.g., ³H, ¹²⁵I, ³⁵S, ¹⁴C, or ³²p), enzymes (e.g., horse radish peroxidase, alkaline phosphatase and others commonly used in an ELISA), and colorimetric labels such as colloidal gold or colored glass or plastic (e.g., polystyrene, polypropylene, latex, etc.) beads.

For example, after incubation with the primary antibodies of the invention, the plates are washed to remove unbound antibody. The plates are then incubated with a secondary antibody that binds to the primary antibody. The secondary antibody has been chemically conjugated to a detectable label, such as peroxidase. Binding of the secondary antibody will be traced by measuring the activity of the detectable label. Specifically, the plates are washed to remove unbound secondary antibody, and the amount of plate-bound secondary antibody is quantified by measuring the activity of the marker enzyme in a standard colorimetric reaction.

As another example, step (c) could include the steps of: (i) separating the proteins and polypeptides after step (b) by gel electrophoresis; (ii) performing an immunoblot of the gel using the antibody (e.g., Western blot); and (iii) measuring the amount of antibody in (ii) that is bound to the biotinylated histone or polypeptide fragment thereof to indicate the amount of biotinyl transferase activity in the biological sample. For example, after incubation with the substrate, the peptides can be electroblotted onto a PDVF membrane; unspecific protein-binding sites will be blocked by incubating the membrane with bovine serum albumin. The membranes are washed, and then incubated with the antibodies of the invention that bind biotinylated histones. The membranes are washed to remove unbound antibody, and the membranes are further incubated with a secondary antibody that binds to the primary antibody of the invention. For example, if the primary antibody has been produced in rabbits, then the secondary antibody will be an anti-rabbit antibody (e.g., from goat). The secondary antibody has been chemically conjugated to a detectable label, such as peroxidase. Finally, the membranes are washed to remove unbound secondary antibody, and the amount of membrane-bound secondary antibody is quantified by measuring the activity of the marker enzyme by chemiluminescence.

One of skill in the art will appreciate that other techniques for combining the components and measuring the biotinylation of the histones or fragments thereof are possible, and any suitable technique is encompassed by the invention. Various techniques can include, but are not limited to, Western blot, immunoblot, enzyme-linked immunosorbant assay (ELISA), radioimmunoassay (RIA), immunoprecipitation, surface plasmon resonance, chemiluminescence, fluorescent polarization, phosphorescence, immunohistochemical analysis, matrix-assisted laser desorption/ionization time-of-flight (MALDI-TOF) mass spectrometry, microcytometry, microarray, microscopy, fluorescence activated cell sorting (FACS), and flow cytometry. This method of the invention is exemplified in the Examples section.

A variation of this method of the invention is a method to identify a compound that regulates the histone biotinylation, such method comprising the steps of: (a) contacting a putative regulatory compound of histone biotinylation with a histone or a polypeptide fragment thereof, wherein the polypeptide fragment thereof comprises at least one biotinylation site in the histone, and wherein the histone or polypeptide fragment thereof is not biotinylated prior to contact with the biological sample; (b) contacting the histone or polypeptide fragment thereof with an enzyme selected from the group consisting of biotinidase and holocarboxylase synthetase, either after step (a) or at the same time as step (a); (c) contacting the histone or polypeptide fragment thereof with a substrate for the enzyme in (b), either after step (b) or at the same time as step (b); and (d) measuring the amount of histone or polypeptide fragment thereof that is biotinylated after step (c). A decrease in the amount of biotinylated histone or polypeptide fragment thereof in the presence of the putative regulatory compound as compared to in the absence of the putative regulatory compound indicates that the putative regulatory compound is an inhibitor of histone biotinylation (and potentially an inhibitor of the enzyme). An increase in the amount of biotinylated histone or polypeptide fragment thereof in the presence of the putative regulatory compound as compared to in the absence of the putative regulatory compound indicates that the putative regulatory compound is an enhancer of histone biotinylation (and potentially an enhancer of the enzyme). Such a method can include in step (c), detecting the amount of biotinylated histones or polypeptide fragments thereof by contacting the histones or polypeptide fragments thereof with an antibody that selectively binds to the histone or polypeptide fragment when the histone or polypeptide fragment is biotinylated and not to non-biotinylated histone or polypeptide fragment thereof. Such a method can also include additional steps of confirming whether the regulator inhibits or enhances the enzyme (biotinidase or holocarboxylase synthetase), such as by using binding assays and/or assays that measure the activity of the enzyme other than the assay described above.

As used herein, the term “test compound”, “putative inhibitory compound” or “putative regulatory compound” refers to compounds having an unknown or previously unappreciated regulatory activity in a particular process. As such, the term “identify” with regard to methods to identify compounds is intended to include all compounds, the usefulness of which as a regulatory compound for the purposes of regulating a biological process associated with the biotinylation of histones is determined by a method of the present invention. A preferred amount of putative regulatory compound(s) to contact with a sample according to the invention can comprise between about 1 nM to about 10 mM of putative regulatory compound(s) per well of a 96-well plate. The invention is not limited to these concentrations, as one of skill in the art will be able to determine the appropriate concentration for a given assay condition and type of compound to be tested.

Compounds to be screened in the methods of the invention include known organic compounds such as peptides (e.g., products of peptide libraries), oligonucleotides, nucleotides, carbohydrates, synthetic organic molecules (e.g., products of chemical combinatorial libraries), and antibodies. Compounds may also be identified using rational drug design relying on the structure of the product of a gene or polynucleotide. Such methods are known to those of skill in the art and involve the use of three-dimensional imaging software programs. For example, various methods of drug design, useful to design or select mimetics or other therapeutic compounds useful in the present invention are disclosed in Maulik et al., 1997, Molecular Biotechnology: Therapeutic Applications and Strategies, Wiley-Liss, Inc., which is incorporated herein by reference in its entirety.

As used herein, a mimetic, which may be a putative regulatory compound, refers to any peptide or non-peptide compound that is able to mimic the biological action of a naturally occurring peptide, often because the mimetic has a basic structure that mimics the basic structure of the naturally occurring peptide and/or has the salient biological properties of the naturally occurring peptide. Mimetics can include, but are not limited to: peptides that have substantial modifications from the prototype such as no side chain similarity with the naturally occurring peptide (such modifications, for example, may decrease its susceptibility to degradation); anti-idiotypic and/or catalytic antibodies, or fragments thereof; non-proteinaceous portions of an isolated protein (e.g., carbohydrate structures); or synthetic or natural organic molecules, including nucleic acids and drugs identified through combinatorial chemistry, for example. Such mimetics can be designed, selected and/or otherwise identified using a variety of methods known in the art.

A mimetic can be obtained, for example, from molecular diversity strategies (a combination of related strategies allowing the rapid construction of large, chemically diverse molecule libraries), libraries of natural or synthetic compounds, in particular from chemical or combinatorial libraries (i.e., libraries of compounds that differ in sequence or size but that have the similar building blocks) or by rational, directed or random drug design. See for example, Maulik et al., supra.

In a molecular diversity strategy, large compound libraries are synthesized, for example, from peptides, oligonucleotides, carbohydrates and/or synthetic organic molecules, using biological, enzymatic and/or chemical approaches. The critical parameters in developing a molecular diversity strategy include subunit diversity, molecular size, and library diversity. The general goal of screening such libraries is to utilize sequential application of combinatorial selection to obtain high-affinity ligands for a desired target, and then to optimize the lead molecules by either random or directed design strategies. Methods of molecular diversity are described in detail in Maulik, et al., ibid.

Maulik et al. also disclose, for example, methods of directed design, in which the user directs the process of creating novel molecules from a fragment library of appropriately selected fragments; random design, in which the user uses a genetic or other algorithm to randomly mutate fragments and their combinations while simultaneously applying a selection criterion to evaluate the fitness of candidate ligands; and a grid-based approach in which the user calculates the interaction energy between three dimensional receptor structures and small fragment probes, followed by linking together of favorable probe sites.

Designing a compound for testing in a method of the present invention can include creating a new chemical compound or searching databases of libraries of known compounds (e.g., a compound listed in a computational screening database containing three dimensional structures of known compounds). Designing can also be performed by simulating chemical compounds having substitute moieties at certain structural features. The step of designing can include selecting a chemical compound based on a known function of the compound. A preferred step of designing comprises computational screening of one or more databases of compounds in which the three dimensional structure of the compound is known and is interacted (e.g., docked, aligned, matched, interfaced) with the three dimensional structure of a target by computer (e.g. as described by Humblet and Dunbar, Animal Reports in Medicinal Chemistry, vol. 28, pp. 275-283, 1993, M Venuti, ed., Academic Press). Methods to synthesize suitable chemical compounds are known to those of skill in the art and depend upon the structure of the chemical being synthesized. Methods to evaluate the bioactivity of the synthesized compound depend upon the bioactivity of the compound (e.g., inhibitory or stimulatory).

Candidate compounds identified or designed by the methods of the invention can be synthesized using techniques known in the art, and depending on the type of compound. Synthesis techniques for the production of non-protein compounds, including organic and inorganic compounds are well known in the art. For example, for smaller peptides, chemical synthesis methods are preferred. For example, such methods include well known chemical procedures, such as solution or solid-phase peptide synthesis, or semi-synthesis in solution beginning with protein fragments coupled through conventional solution methods. Such methods are well known in the art and may be found in general texts and articles in the area such as: Merrifield, 1997, Methods Enzymol. 289:3-13; Wade et al., 1993, Australas Biotechnol. 3(6):332-336; Wong et al., 1991, Experientia 47(11-12):1123-1129; Carey et al., 1991, Ciba Found Symp. 158:187-203; Plaue et al., 1990, Biologicals 18(3):147-157; Bodanszky, 1985, Int. J. Pept. Protein Res. 25(5):449-474; or H. Dugas and C. Penney, BIOORGANIC CHEMISTRY, (1981) at pages 54-92, all of which are incorporated herein by reference in their entirety. For example, peptides may be synthesized by solid-phase methodology utilizing a commercially available peptide synthesizer and synthesis cycles supplied by the manufacturer. One skilled in the art recognizes that the solid phase synthesis could also be accomplished using an FMOC strategy and a TFA/scavenger cleavage mixture. A compound that is a protein or peptide can also be produced using recombinant DNA technology and methods standard in the art, particularly if larger quantities of a protein are desired.

Techniques for performing these steps of this method of the invention are largely as described for the method to detect biotinyl transferase activity described above. Biotinidase and holocarboxylase synthetase (HCS) are well known in the art and can be purchased commercially or produced recombinantly.

In this aspect of the invention, a putative regulatory compound is selected as a regulator of biotinylation of histones if the compound causes a statistically significant (p<0.05) inhibition or enhancement of the biotinylation of the histones or fragments thereof as compared to in the absence of the putative regulatory compound.

If a suitable regulatory compound is identified using the methods described herein, a composition can be formulated, including a therapeutic composition. A composition, and particularly a therapeutic composition, of the present invention generally includes the therapeutic compound and a carrier, and preferably, a pharmaceutically acceptable carrier. According to the present invention, a “pharmaceutically acceptable carrier” includes pharmaceutically acceptable excipients and/or pharmaceutically acceptable delivery vehicles, which are suitable for use in administration of the composition to a suitable in vitro, ex vivo or in vivo site. Preferred pharmaceutically acceptable carriers are capable of maintaining a compound, a protein, a peptide, nucleic acid molecule or mimetic (drug) in a form that, upon arrival of the compound, protein, peptide, nucleic acid molecule or mimetic at the target site in a culture (in the case of an in vitro or ex vivo protocol) or in patient (in vivo), the compound, protein, peptide, nucleic acid molecule or mimetic is capable of providing the desired effect at the target site.

Suitable excipients of the present invention include excipients or formularies that transport or help transport, but do not specifically target a composition to a cell (also referred to herein as non-targeting carriers). Examples of pharmaceutically acceptable excipients include, but are not limited to water, phosphate buffered saline, Ringer's solution, dextrose solution, serum-containing solutions, Hank's solution, other aqueous physiologically balanced solutions, oils, esters and glycols. Aqueous carriers can contain suitable auxiliary substances required to approximate the physiological conditions of the recipient, for example, by enhancing chemical stability and isotonicity.

One type of pharmaceutically acceptable carrier includes a controlled release formulation that is capable of slowly releasing a composition of the present invention into a patient or culture. As used herein, a controlled release formulation comprises a therapeutic compound in a controlled release vehicle. Suitable controlled release vehicles include, but are not limited to, biocompatible polymers, other polymeric matrices, capsules, microcapsules, microparticles, bolus preparations, osmotic pumps, diffusion devices, liposomes, lipospheres, and transdermal delivery systems. Other carriers include liquids that, upon administration to a patient, form a solid or a gel in situ. Preferred carriers are also biodegradable (i.e., bioerodible). When the compound is a recombinant nucleic acid molecule, suitable delivery vehicles include, but are not limited to liposomes, viral vectors or other delivery vehicles, including ribozymes. Natural lipid-containing delivery vehicles include cells and cellular membranes. Artificial lipid-containing delivery vehicles include liposomes and micelles. A delivery vehicle of the present invention can be modified to target to a particular site in a patient, thereby targeting and making use of a therapeutic compound at that site. Suitable modifications include manipulating the chemical formula of the lipid portion of the delivery vehicle and/or introducing into the vehicle a targeting agent capable of specifically targeting a delivery vehicle to a preferred site, for example, a preferred cell type. Other suitable delivery vehicles include gold particles, poly-L-lysine/DNA-molecular conjugates, and artificial chromosomes.

A compound or composition can be delivered to a cell culture or patient by any suitable method. Selection of such a method will vary with the type of compound being administered or delivered (i.e., compound, protein, peptide, nucleic acid molecule, or mimetic), the mode of delivery (i.e., in vitro, in vivo, ex vivo) and the goal to be achieved by administration/delivery of the compound or composition. According to the present invention, an effective administration protocol (i.e., administering a composition in an effective manner) comprises suitable dose parameters and modes of administration that result in delivery of a composition to a desired site (i.e., to a desired cell) and/or in the desired regulatory event.

Administration routes include in vivo, in vitro and ex vivo routes. In vivo routes include, but are not limited to, oral, nasal, intratracheal injection, inhaled, transdermal, rectal, and parenteral routes. Preferred parenteral routes can include, but are not limited to, subcutaneous, intradermal, intravenous, intramuscular and intraperitoneal routes. Intravenous, intraperitoneal, intradermal, subcutaneous and intramuscular administrations can be performed using methods standard in the art. Aerosol (inhalation) delivery can also be performed using methods standard in the art (see, for example, Stribling et al., Proc. Natl. Acad. Sci. USA 189:11277-11281, 1992, which is incorporated herein by reference in its entirety). Oral delivery can be performed by complexing a therapeutic composition of the present invention to a carrier capable of withstanding degradation by digestive enzymes in the gut of an animal. Examples of such carriers, include plastic capsules or tablets, such as those known in the art. Direct injection techniques are particularly useful for suppressing graft rejection by, for example, injecting the composition into the transplanted tissue, or for site-specific administration of a compound, such as at the site of a tumor. Ex vivo refers to performing part of the regulatory step outside of the patient, such as by transfecting a population of cells removed from a patient with a recombinant molecule comprising a nucleic acid sequence encoding a protein according to the present invention under conditions such that the recombinant molecule is subsequently expressed by the transfected cell, and returning the transfected cells to the patient. In vitro and ex vivo routes of administration of a composition to a culture of host cells can be accomplished by a method including, but not limited to, transfection, transformation, electroporation, microinjection, lipofection, adsorption, protoplast fusion, use of protein carrying agents, use of ion carrying agents, use of detergents for cell permeabilization, and simply mixing (e.g., combining) a compound in culture with a target cell.

A compound, as well as compositions comprising such compounds, can be administered to any organism, and particularly, to any member of the Vertebrate class, Mammalia, including, without limitation, primates, rodents, livestock and domestic pets. Preferred mammals include humans. Typically, it is desirable to obtain a therapeutic benefit in a patient. A therapeutic benefit is not necessarily a cure for a particular disease or condition, but rather, preferably encompasses a result which can include alleviation of the disease or condition, elimination of the disease or condition, reduction of a symptom associated with the disease or condition, prevention or alleviation of a secondary disease or condition resulting from the occurrence of a primary disease or condition, and/or prevention of the disease or condition. As used herein, the phrase “protected from a disease” refers to reducing the symptoms of the disease; reducing the occurrence of the disease, and/or reducing the severity of the disease. Protecting a patient can refer to the ability of a composition of the present invention, when administered to a patient, to prevent a disease from occurring and/or to cure or to alleviate disease symptoms, signs or causes. As such, to protect a patient from a disease includes both preventing disease occurrence (prophylactic treatment) and treating a patient that has a disease (therapeutic treatment) to reduce the symptoms of the disease. A beneficial effect can easily be assessed by one of ordinary skill in the art and/or by a trained clinician who is treating the patient. The term, “disease” refers to any deviation from the normal health of a mammal and includes a state when disease symptoms are present, as well as conditions in which a deviation (e.g., infection, gene mutation, genetic defect, etc.) has occurred, but symptoms are not yet manifested.

Another embodiment of the invention relates to an assay to detect debiotinylase activity in a biological sample. This embodiment includes the steps of: (a) incubating a biological sample with a biotinylated histone or a biotinylated polypeptide fragment thereof according to the present invention; (b) contacting the biological sample and biotinylated histone or fragment thereof with an avidin-conjugated detectable label; and (c) measuring the amount of avidin-conjugated detectable label that is bound to the biotinylated histone or fragment thereof after incubation with the biological sample as compared to prior to the incubation step. In this embodiment, an amount of reduction in the biotinylation of the histone or fragment thereof after the incubation step indicates the amount of debiotinylase activity in the biological sample. This method is described in detail in Example 4. In addition, the steps of contacting, incubating and measuring have been generally described above with regard to other methods of the invention. The specificity of this method can be enhanced through the use of the polypeptides comprising biotinylation sites and antibodies described herein. For example, the biotinylated polypeptide fragments of histones described herein can be used in place of a complete histone. In addition, the antibodies of the invention can be used to measure debiotinylation in place of the avidin-conjugated detectable label (e.g., by measuring a decrease in antibody binding as compared to the beginning of the assay). Other variations will be apparent to those of skill in the art.

Various aspects of the present invention are described in the following experiments. These experimental results are for illustrative purposes only and are not intended to limit the scope of the present invention.

### EXAMPLES

### Example 1

The following example demonstrates the identification of residues that are biotinylated in histone H4, antibodies that bind to such sites, and shows that acetylation and methylation of histone H4 regulate biotinylation in histone H4.

Materials And Methods

Peptide Synthesis

Previous studies have suggested that lysine residues in histone H4 are likely targets for biotinylation (Zempleni and Mock, 1999). Here, synthetic peptides spanning fragments of human histone H4 (GenBank accession number NM_{—}175054; amino acid sequence represented herein by SEQ ID NO:6) were used to identify lysines that are targets for biotinylation. Peptides were synthesized using N-fluoren-9-ylmethoxycarbonyl (Fmoc) chemistry by a standard solid-phase method (Fields, 1998). One-letter annotation is used for denoting amino acids throughout this example (Garrett and Grisham, 1995). All solvents were purchased from EM Science (Gibbstown, N.J.) unless noted otherwise. L-isomers of Fmoc-amino acids (25 mg/coupling; Ana Spec Inc, San Jose, Calif.) were used for peptide synthesis unless noted otherwise. Chemically modified peptides were synthesized by using biotinylated, acetylated, dimethylated, or formylated ε-NH2-derivatives of Fmoc-lysine. For pilot studies, the following two peptides were synthesized using a Pioneer peptide synthesizer (ABI Inc, Foster City, Calif.) using manufacturer recommended protocols: (i) 12 N-terminus of histone H4, spanning amino acids 1 through 19 (SGRGKGGKGLGKGGAKRHR; SEQ ID NO:7); the N-terminus contains lysines in position 5, 8, 12, and 16; (ii) C-terminus of histone H4, spanning amino acids 82 through 102 (TAMDVVYALKRQGRTLYGFGG; SEQ ID NO:8). For peptide analogs, a base peptide of the sequence, Fmoc-GGABBRC-amide (SEQ ID NO:9), was assembled on PAL resin (ABI Inc, Foster City, Calif.; B=beta-alanine) using a Pioneer peptide synthesizer (ABI Inc, Foster City, Calif.). Aliquots of approximately 25 mg of the base resin (˜20 μmol of peptide) were used to manually synthesize the different H4 peptide analogs using established procedures (Sigal et al., 1995; Smit et al., 2003). A majority of the studies described below focused on the N-terminus in histone H4, based on the following lines of reasoning: (i) Pilot studies suggested that the N-terminus of histone H4 is a good target for biotinylation whereas the C-terminus is not (see below); (ii) lysine residues in the N-termini of histones are likely targets for biotinylation (Zempleni and Mock, 1999); (iii) lysines 8 and 12 in histone H4 are less likely to be occupied by acetylation than lysine-16 (Smit et al., 2003); this is consistent with the availability of lysine-8 and lysine-12 for biotinylation. Thus, the majority of these studies were based on using the following H4 fragment and variations thereof: GGK(8)GLGK(12)GGA (SEQ ID NO:10)(“K” denotes lysines in position 8 and 12, respectively, in the H4 molecule); modifications were introduced in positions 8 and 12 during peptide synthesis (Table 1).

Peptide Quantification

Lyophilized peptides were dissolved in 2 mL of distilled water, and quantified based on their cysteine residue using Ellman's reagent (Ellman, 1958). Briefly, aliquots (20 μL) of peptide solutions were mixed with 178 μL of 1.0 mol/L Tris (pH 8.2) containing 0.02 mol/L EDTA, and with 2 μL of 0.01 mol/L 5,5′-dithiobis-2-nitrobenzoic acid in methanol. Cysteine standard curves (0-1.14 mol/L) were used for calibration. Samples were incubated for 10 min at room temperature and absorbance was measured at 405 nm. Equal amounts of peptides were used in subsequent biotinylation experiments.

Enzymatic Biotinylation of Peptides

It has been proposed that the following catalytic sequence leads to biotinylation in histones (Hymes et al., 1995; Hyme and Wolf, 1999). First, biocytin is cleaved by biotinidase to form an intermediate, cysteine-bound biotin. Second, the biotinyl moiety from the cysteine residue is transferred to the ε-amino group of lysines in histones. In the present study, synthetic peptides were biotinylated enzymatically using human plasma (as source of biotinidase) and biocytin (as source of biotin) as described previously (Hymes et al., 1995). Peptide concentrations in stock solutions were adjusted to 50 mg/L; 20 μL of peptide solution was mixed with 1.88 mL of 50 mmol/L Tris (pH 8.0), 40 μL of 0.75 mmol/L biocytin and 60 μL of human plasma. Samples were incubated 1 d at 37° C. for 45 min and stored at −70° C. unless stated otherwise.

Gel Electrophoresis

After enzymatic biotinylation, peptides were electrophoresed using 16% tricine polyacrylamide gels according to the manufacturer's instructions (Invitrogen, Carlsbad, Calif.). Peptides were electroblotted onto polyvinylidene fluoride membranes (Millipore, Bedford, Mass.), which were blocked with 50 mL of 30 g/L BSA. Peptide-bound biotin was probed with streptavidin peroxidase (Stanley et al., 2001).

HPLC Analysis

Peptides were chromatographed by HPLC (Shimadzu, Columbia, Md.) (i) to determine purity of synthetic peptides; (ii) to prepare samples for analysis by mass spectrometry; and (iii) to confirm enzymatic biotinylation of peptides. Synthetic peptides were chromatographed by HPLC, using a 0.46×25 cm C18 column and the following binary gradient system (buffer A=0.001 L trifluoroacetic acid/1 L water; buffer B=0.001 L trifluoroacetic acid/0.9 L acetonitrile/0.1 L water): 85% A and 15% B for 2 min; linear increase to 100% of buffer B over 12 min; 100% of buffer B for 3 min; linear decrease to 15% of buffer B over 3 min; 85% of buffer A and 15% of buffer B for 5 min. Flow rate was 1.0 mL/min. Peptides in the eluate were monitored at 220 nm, using a diode array detector (SPD18M10Avp, Shimadzu).

Mass Spectrometry

Purified peptides were analyzed by matrix assisted laser desorption ionization-time of flight as well as by quadrupole-time of flight mass spectrometry at the University of Nebraska-Lincoln mass spectrometry facility.

Polyclonal Antibody

A polyclonal antibody to human H4 (biotinylated at lysine-12) was generated using a commercial facility (Cocalico Biologicals, Reamstown, Pa.). This antibody was used to detect biotinylated histone H4 in human cells. Briefly, a conjugate of a synthetic peptide, biotinylated at lysine-12, (peptide 11 in Table 1) and keyhole limpet hemocyanin was injected into white New Zealand rabbits. Booster injections were given after 14, 21, and 49 days. Serum was collected before immunization and 2 days after each booster injection. Pre-immunization serum did not bind to histones in Western blot analysis (data not shown); serum collected after the second and third booster injection were used for assays described below. First, the inventors determined whether the antibody was specific for biotinylation sites. Electroblots of synthetic peptides (biotinylated at either lysine-8 or lysine-12) were probed with the anti-histone H4 (biotinylated at lysine-12) antibody and a polyclonal goat anti-rabbit IgG peroxidase conjugate (Griffin et al., 2002). Second, the inventors determined whether human cells contain histone H4, biotinylated at lysine-12. Nuclear histones were extracted from human lymphoid (Jurkat) cells (Peters et al., 2002) using hydrochloric acid (Stanley et al., 2001). Extracts were electrophoresed using 18% Tris-Glycine polyacrylamide gels (Invitrogen) as described (Stanley et al., 2001). Biotinylated histone H4 was probed using the anti-histone H4 (biotinylated at lysine-12) antibody using standard procedures (Griffin et al., 2002). Biotin-free controls for Western blot analysis were prepared as follows: 0.1 mL of histone extract (approximately 0.5 mg of histones) were incubated with 0.05 mL of avidin beads (Pierce; Rockford, Ill.) at 4° C. for 1 h. The supernatant did not contain detectable quantities of biotinylated histones, as judged by probing with streptavidin-peroxidase (Stanley et al., 2001); treatment with avidin beads decreased the amount of total histones in the extract by about 50%, as judged by staining with Coomassie blue (Stanley et al., 2001).

Results

Biotinylation Sites in Histone H4

First, the inventors determined whether the N-terminus or the C-terminus of histone H4 is a better substrate for biotinylation by biotinidase. Peptides spanning the N-terminal 19 amino acids (SGRGKGGKGLGKGGAKRHR; SEQ ID NO:7) and the C-terminal 21 amino acids (TAMDVVYALKRQGRTLYGFGG; SEQ ID NO:8) of histone H4 were incubated with biotinidase and biocytin for enzymatic biotinylation. The N-terminal peptide was biotinylated by biotinidase, whereas the C-terminal peptide was not biotinylated; controls incubated without biocytin and biotinidase did not produce a detectable signal (data not shown). This is consistent with the hypothesis that the N-terminal tail of histone H4 contains a biotinylation motif that is not present in the C-terminal domain. Subsequent studies focused on peptides derived from the N-terminus of histone H4.

A time course was conducted to determine when biotinylation of peptides reaches maximal levels. The N-terminal peptide (SGRGKGGKGLGKGGAKRHR; SEQ ID NO:7) was incubated with plasma and biocytin for 0 (control), 2, 4, 8, 12, 16, 20, 30, 40, 50, and 60 min. Abundance of biotinylated peptide reached a plateau 20-60 min after starting the reaction (data not shown). All subsequent enzymatic biotinylations were conducted for 45 minutes.

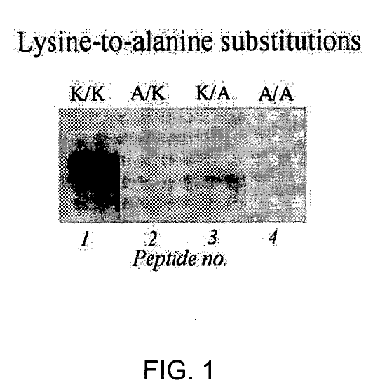

For reasons described above, the inventors focused on lysine residues 8 and 12 in histone H4 when investigating biotinylation sites. The following peptide spans amino acids 6 through 15 in histone H4, and was used as a native control: GGKGLGKGGA (SEQ ID NO:10) (peptide 1 in Table 1). This peptide was efficiently biotinylated by biotinidase, suggesting that lysines in position 8 or 12 (or both) are targets for biotinylation (FIG. 1, peptide 1, SEQ ID NO:10, “K/K”). If one of the lysines in position 8 or 12 was replaced by an alanine (peptides 2 (SEQ ID NO:11) and 3 (SEQ ID NO:12), respectively in Table 1), the covalent binding of biotin decreased substantially (FIG. 1; “A/K” and “K/A”); deletion of lysine-8 had a greater effect than deletion of lysine-12. When both lysines were replaced by alanines (peptide 4 (SEQ ID NO:13) in Table 1), the synthetic peptide did not undergo biotinylation (FIG. 1; “A/A”). Collectively, these data suggest that both lysines 8 and 12 are targets for biotinylation, and that lysine-8 seems to be a better target for biotinylation by biotinidase than lysine-12.

TABLE 1

| | Position^{b} | | | | | | | | | | |
|---|---|---|---|---|---|---|---|---|---|---|---|
| | | | | | | | | | | | SEQ- |
| Pep- | | | | | | | | | | | ID |
| tide | 6 | 7 | 8 | 9 | 10 | 11 | 12 | 13 | 14 | 15 | NO: |
| 1 | _{Ac}-G | G | K | G | L | G | K | G | G | A | 10 |
| 2 | _{Ac}-G | G | A | G | L | G | K | G | G | A | 11 |
| 3 | _{Ac}-G | G | K | G | L | G | A | G | G | A | 12 |
| 4 | _{Ac}-G | G | A | G | L | G | A | G | G | A | 13 |
| 5 | _{Ac}-G | G | _{Ac}-K | G | L | G | K | G | G | A | 14 |
| 6 | _{Ac}-G | G | K | G | L | G | _{Ac}-K | G | G | A | 15 |
| 7 | _{Ac}-G | G | _{Dme}-K | G | L | G | K | G | G | A | 16 |
| 8 | _{Ac}-G | G | K | G | L | G | _{Dme}-K | G | G | A | 17 |
| 9 | _{Ac}-G | G | K | G | L | G | _{For}-K | G | G | A | 18 |
| 10 | _{Ac}-G | G | _{Bio}-K | G | L | G | K | G | G | A | 19 |
| 11 | _{Ac}-G | G | K | G | L | K | _{Bio}-K | G | G | A | 20 |
| 12 | _{Ac}-G | G | R | G | L | G | K | G | G | A | 21 |
| 13 | _{Ac}-G | G | K | G | L | G | R | G | G | A | 22 |
| 14 | _{Ac}-G | G | R | G | L | G | R | G | G | A | 23 |
| 15 | _{Ac}-G | G | E | G | L | G | K | G | G | A | 24 |
| 16 | _{Ac}-G | G | K | G | L | G | E | G | G | A | 25 |
| 17 | _{Ac}-G | G | Q | G | L | G | K | G | G | A | 26 |
| 18 | _{Ac}-G | G | K | G | L | G | Q | G | G | A | 27 |
| 19 | _{Ac}-G | G | Q | G | L | G | Q | G | G | A | 28 |
| 20 | _{Ac}-G | G | K | G | L | G | _{D}-K | G | G | A | 29 |
| ^{a}One-letter amino acid code and abbreviations: A, _{L}-alanine; _{Ac}-G, acetyl-α-NH₂-_{L}-glycine; _{Ac}-K, acetyl-ε-NH₂-_{L}-lysine; bio-K, | | | | | | | | | | | |
| # biotin-ε-NH₂-_{L}-lysine; _{D}-K, _{D}-lysine; Dme-K, | | | | | | | | | | | |
| # dimethyl-ε-NH₂-_{L}-lysine; E, _{L}-glutamate; For-K, formyl-ε-NH₂-_{L}-lysine; G, _{L}-glycine; K, _{L}-Lysine; L, _{L}-leucin; | | | | | | | | | | | |
| # Q, _{L}-glutamine; R, _{L}-arginine. Deviations from the native sequence (peptide 1) are in bold. | | | | | | | | | | | |

^{b}Numbers refer to the positions of amino acids in human histone H4 (GenBank accession number NM_175054) after removal of the N-terminal methionine.

Effects of Amino Acid Modifications in Positions 8 and 12

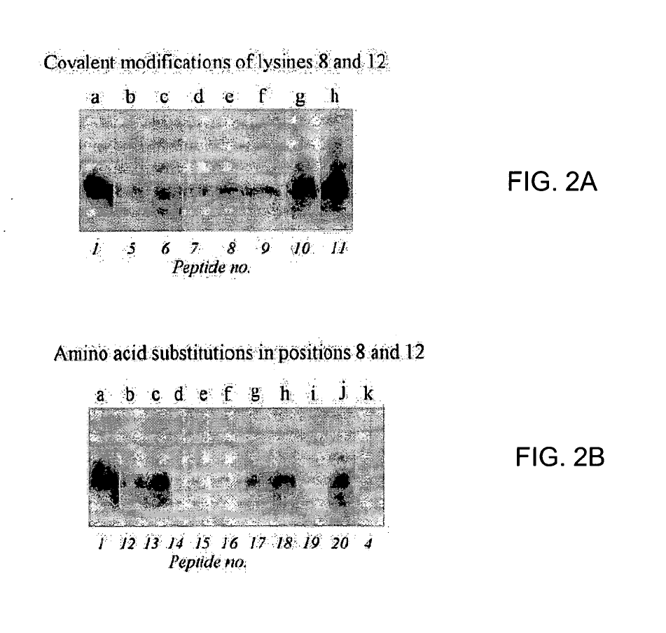

Biotinylation of lysines-8 and -12 decreases if neighboring lysine residues were covalently modified by acetylation, formylation, or dimethylation. If lysine-8 was acetylated (peptide 5 (SEQ ID NO:14) in Table 1), biotinylation was barely detectable (FIG. 2A, lane b) compared to the native peptide (SEQ ID NO:10; FIG. 2A, lane a). Likewise, acetylation of lysine-12 (peptide 6 (SEQ ID NO:15) in Table 1) decreased biotinylation of the peptide (FIG. 2A, lane c). If one of the lysines in position 8 or 12 was dimethylated (peptides 7 (SEQ ID NO:16) and 8 (SEQ ID NO:17), respectively in Table 1), covalent modification by biotin decreased substantially (FIG. 2A, lanes d and e). When lysine-12 was replaced by formyl-lysine (peptide 9 (SEQ ID NO: 18) in Table 1), biotinylation of lysine-8 decreased compared to the native peptide (FIG. 2A, lane f). Lanes g and h in FIG. 2A depict synthetic peptides that were chemically biotinylated in positions -8 or -12 (peptides 10 (SEQ ID NO: 19) and 11 (SEQ ID NO:20), respectively in Table 1).

Previous studies provided preliminary evidence that guanidino groups in arginine residues are not good targets for biotinylation (Zempleni and Mock, 1999). This was confirmed in the present study: if lysine-8 was replaced by arginine (peptide 12 (SEQ ID NO:21) in Table 1) efficiency of enzymatic biotinylation decreased substantially (FIG. 2B, compare lanes “a” and “b”). Similarly, if lysine-12 was replaced by arginine (peptide 13 (SEQ ID NO:22) in Table 1), efficiency of biotinylation decreased moderately (FIG. 2B, compare lanes “a” and “c”). Finally, if both lysine-8 and lysine-12 were replaced by arginines (peptide 14 (SEQ ID NO:23) in Table 1), biotinylation was not detected (FIG. 2B, lane d).

Covalent modifications of histones can change the net charge of the molecule, e.g., phosphorylation and poly (ADP-ribosylation) introduce negative charges and subsequently influence other post-translational modifications of nearby residues (Wolffe, 1998; Jenuwein and Allis, 2001; Strahl and Allis, 2000). Theoretically, localized changes in charge could affect biotinylation of histones. To verify this scenario, lysine residues were substituted by glutamates to introduce negative charges into synthetic peptides. If lysine-8 (peptide 15 (SEQ ID NO:24) in Table 1) or lysine-12 (peptide 16 (SEQ ID NO:25) in Table 1) was replaced by glutamate, enzymatic biotinylation was not detectable (FIG. 2B, lanes e and f, respectively). Next, the inventors sought to formally exclude the possibility that effects of glutamate were caused by steric hindrance rather than by charge effects. Glutamine is of similar size as glutamate but does not carry a net charge. Thus, lysine-8 or lysine-12 were replaced with glutamine (peptide 17 (SEQ ID NO:26) and 18 (SEQ ID NO:27), respectively in Table 1). Enzymatic biotinylation of glutamine-substituted peptides decreased compared to the native peptide (FIG. 2A, compare lanes g and h to lane a), but effects of glutamine substitution were smaller than effects of glutamate substitution. If both lysines-8 and -12 were replaced with glutamine (negative control), no enzymatic biotinylation was detectable (FIG. 2B, lane i; peptide 19 (SEQ ID NO:28) in Table 1). These results suggest that charge interactions between histones and biotinidase are important for enzymatic biotinylation.

Biotinylation of lysine residues in histones is not stereospecific. If L-lysine in position 12 was replaced with D-lysine, enzymatic biotinylation decreased only moderately (FIG. 2B, lane j; peptide 20 (SEQ ID NO:29) in Table 1) compared with the native peptide (FIG. 2B, lane a). Lane k in FIG. 2B depicts a peptide where both lysines were replaced by alanine (negative control).

Identification of Biotinylated Peptides by HPLC/MS

Analysis of peptides incubated with biotinidase and biocytin by HPLC/mass spectrometry confirmed that biotinidase mediated covalent biotinylation. First, an HPLC method was developed to separate non-biotinylated peptides from biotinylated peptides. Non-biotinylated peptide derived from the N-terminus in histone H4 (e.g., peptide 1 (SEQ ID NO: 10) in Table 1) eluted at t=6.0 min; peptides that were chemically biotinylated at either lysine-8 (peptide 10 in Table 1) or lysine-12 (peptide 11 in Table 1) eluted at t=9.5 min (data not shown). This is consistent with a decreased polarity of biotinylated peptides compared to non-biotinylated controls. HPLC fractions eluting at 6 min (native peptide) and 9.5 min (biotinylated peptide) were analyzed by mass spectrometry at the Nebraska Center for Mass Spectrometry, University of Nebraska-Lincoln. Molecules of the following masses were detected: 1243.4 for the native, non biotinylated peptide (expected mass=1242.6) and 1469.8 for the chemically biotinylated peptides (expected mass=1468.6). These data confirmed the identities of synthetic peptides.

Next, the native, non-biotinylated peptide derived from the N-terminus in histone H4 (peptide 1 in Table 1) was incubated with biocytin and biotinidase before separation by HPLC. The HPLC fraction eluting at 9.5 min was collected and subjected to mass spectrometry as described above. A molecule with a mass of 1469.7 daltons was detected, confirming enzymatic biotinylation of the peptide.

Polyclonal Antibody

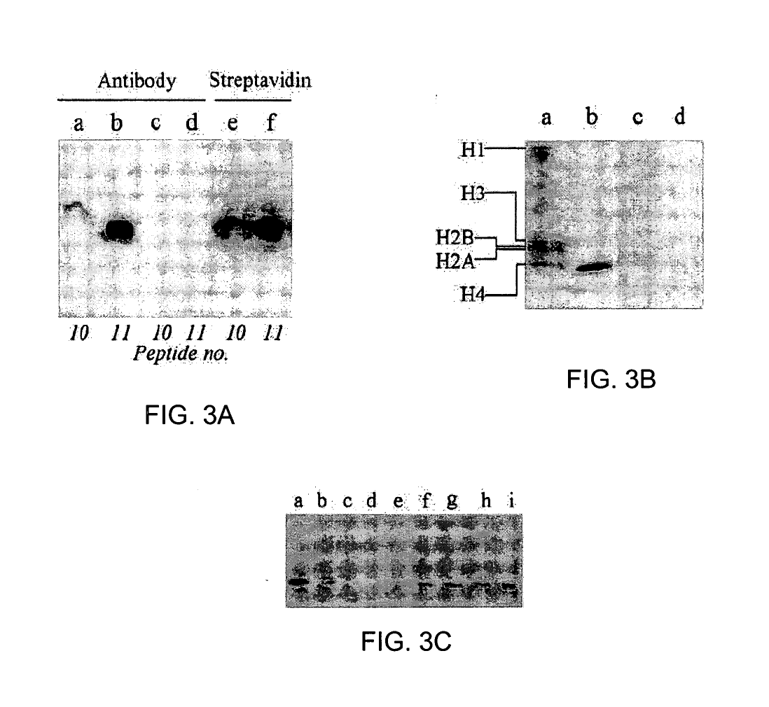

A polyclonal antibody was generated to determine whether histone H4 is biotinylated at lysine-12 in human cell nuclei. First, the inventors determined whether the antibody was specific for biotinylation sites. Transblots of biotinylated peptides 10 and 11 (Table 1) were probed with the newly synthesized antibody. The antibody bound to the peptide that was chemically biotinylated at lysine-12, but did not bind to the peptide biotinylated at lysine-8 (FIG. 3A, compare lanes “a” and “b”); both peptides showed similar reactivity when biotin was probed with streptavidin peroxidase (FIG. 3A, compare lanes “c” and “d”). These observations suggest that the two peptides contained biotin, and that the antibody would specifically recognize histone H4, biotinylated at lysine-12. Next, nuclear extracts from Jurkat cells were probed with the antibody. The nuclear extract contained biotinylated histones H1, H2A, H2B, H3 and H4, as judged by staining with streptavidin-peroxidase (FIG. 3B, lane a). The polyclonal antibody bound to histone H4 but did not cross-react with other classes of histones (FIG. 3B, lane b). If biotinylated histones were removed by using avidin beads before electrophoresis, the antibody did not bind to the remaining non-biotinylated histones (FIG. 3B, lane “c”). Collectively, these findings suggest (i) that human cells contain histone H4, biotinylated at lysine-12; (ii) that the present inventors' antibody is specific for histone H4 and does not cross-react with other classes of histones; and (iii) that this antibody does not cross react with non-biotinylated histone H4.

Discussion

This study provides the first evidence (i) that lysine-8 and lysine-12 in histone H4 are targets for biotinylation by biotinidase; (ii) that the C-terminal region of histone H4 is not a target for biotinylation; (iii) that arginine residues are not likely to be biotinylated; (iv) that charge interactions play an important role in biotinylation; and (v) that acetylation and dimethylation of histones decrease biotinylation of neighboring lysine residues.

Biotinylation of histones is believed to be physiologically meaningful. For example, peripheral blood mononuclear cells respond to proliferation with increased biotinylation of histones as compared to quiescent cells (Stanley et al., 2001). Moreover, biotinylation of histones increases in response to DNA damage caused by UV light in human lymphoid cells (Peters et al., 2002). Finally, evidence has been provided that biotinylated histones are enriched in transcriptionally silent chromatin (Peters et al., 2002). These previous studies were limited to using streptavidin-peroxidase as a probe for biotin. The present study is an important first step in developing antibodies that are specific for biotinylation sites in a given class of histones. The availability of such antibodies will foster future studies of biological functions of biotinylated histones.

This example provides evidence that biotinylation occurs in the N-terminus of histone H4 rather than in the C-terminus. The N-terminus of histone H4 contains lysine residues in positions 5, 8, 12, and 16. These lysines are known to be also targets for covalent acetylation, mediating transcriptional activation of genes (Allfrey et al., 1964; Mathis et al, 1978). Among the four lysine residues in the N-terminus of histone H4, lysine-16 is acetylated more abundantly than lysine-12 and lysine-5; the abundance of acetylated lysine-8 is relatively small (Smith et al., 2003). The present study suggests that some of the same lysines are also targets for biotinylation: lysine-8 and lysine-12. Preliminary studies provided evidence that lysine-5 is also biotinylated (data not shown). Lysine-16 may also be a target for biotinylation. Biotinylated histones are enriched in transcriptionally silent heterochromatin (Peters et al., 2002), whereas acetylated histones are enriched in transcriptionally active euchromatin (Wolffe, 1998). Competition between biotin and acetate for the same binding sites is consistent with the mutually exclusive effects of these modifiers on transcriptional activity of chromatin.

Modifications other than acetylation may also play a role in regulating biotinylation. The present study provides evidence that methylation of histones may down-regulate biotinylation. The in vivo relevance of this observation is under investigation, but this study did not investigate classical methylation sites in histone H4. Finally, evidence indicates that phosphorylation of serine residues decreases biotinylation in histone H3 (see Example 2). The “cross-talk” among histone modifications is expected to be important.

The present study provides strong evidence that lysines-8 and -12 in histone H4 are biotinylated enzymatically in-vitro. However, does biotinylation of lysines in histones also occur in vivo? Previous studies suggested that all five major classes of histones are biotinylated in human cells (Stanley et al., 2001) and in chicken erythrocytes (Peters et al., 2002). The value of these previous studies was limited by the fact that biotinylated histones were probed using streptavidin-peroxidase. This probe is neither specific for a given class of histones, nor is it specific for biotinylation sites within a class. The present study for the first time provides evidence that biotinylation of lysine-12 in human histone H4 occurs in vivo. This conclusion is based on probing nuclear extracts from human lymphoid cells with a novel antibody against biotinylated histone H4.

Human cells maintain normal biotinylation of histones if the biotin concentration in culture medium is low (Manthey et al., 2002); under these conditions, biotinylation of carboxylases is barely detectable. It was proposed that biotin-deficient cells maintain normal biotinylation of histones by increasing the nuclear import of biotinidase (Manthey et al., 2002). Alternatively, nuclear accumulation of holocarboxylase synthetase (Narang et al., 2004) or slow turnover of biotinylated histones (Ballard et al., 2002) may contribute to maintaining biotinylation of histones in biotin-deficient cells. The present inventors are knocking down expression of the genes encoding biotinidase and holocarboxylase synthetase. These studies will provide information regarding the roles for these enzymes in maintaining biotinylation of histones in human cells.

### Example 2

The following example demonstrates the identification of residues that are biotinylated in histone H3 and antibodies that bind to such sites, and further demonstrates crosstalk between biotinylation of histones and other known modifications of histones.

Materials and Methods

Peptide Synthesis

Synthetic peptides were used as substrates for biotinidase to identify biotinylation sites in histone H3; the amino acid sequences in these peptides were based on human histone H3 (GenBank accession number NP_{—}066403; amino acid sequence represented herein by SEQ ID NO:5). Peptides were synthesized using N-fluoren-9-ylmethoxycarbonyl (Fmoc) chemistry by a standard solid-phase method (Fields, 1998) as described in Example 1; L-isomers of amino acids were used in all syntheses. One-letter annotation is used for denoting amino acids throughout this example (Garrett and Grisham, 1995). Chemically modified peptides were synthesized by using biotinylated, dimethylated, and phosphorylated Fmoc-ε-NH₂-D-biotinyl-L-lysine, Fmoc-dimethyl-L-arginine, and Fmoc-phospho-L-serine. Identities of synthetic peptides were confirmed by using mass spectrometry (see Example 1).

Posttranslational modifications of histone H3 cluster in the N-terminal region of the molecule (amino acids 1 to 36), e.g., methylation of K4 and K9, acetylation of K9, K18, K23, and K36, phosphorylation of S10, and mono- or dimethylation of R17 (Fischle et al., 2003). In pilot studies the following synthetic peptides were used to determine whether biotinylation of histone H3 also takes place in the N-terminal region: (i) N-terminus of histone H3, spanning amino acids 1 to 25 (ARTKQTARKSTGGKAPRKQLATKAA (SEQ ID NO:30); this peptide was denoted “N₁₋₂₅”), and (ii) a peptide based on amino acids 15 to 39 in histone H3 (APRKQLATKAARKSAPATGGVKKPH (SEQ ID NO:31); denoted “N₁₅₋₃₉”). As a negative control, a peptide spanning the C-terminus of histone H3 was used, i.e., amino acids 116 to 136 (KRVTIMPKDIQLARRIRGERA (SEQ ID NO:32); denoted “C₁₁₆₋₁₃₆”). Pilot studies using these peptides and previous studies of histone H4 (Example 1) suggested that lysines located in the N-terminus of histone H3 are the primary targets for biotinylation (see below). Thus, the studies presented below focused on lysine residues in the N-terminal region; the amino acid sequences of the synthetic peptides used to identify biotinylation sites are provided below.

Enzymatic Biotinylation of Peptides

Synthetic peptides were incubated with biotinidase for enzymatic biotinylation as described previously (Example 1 and Humes et al., 1995); biocytin (biotinyl-ε-lysine) was used as a biotin donor.

Gel Electrophoresis

After enzymatic biotinylation, peptides were resolved using 16% tricine polyacrylamide gels according to the manufacturer's instructions (Invitrogen, Carlsbad, Calif.). Peptides were electroblotted onto polyvinylidene fluoride membranes (Millipore, Bedford, Mass.); peptide-bound biotin was probed with streptavidin-peroxidase (Stanley et al., 2001; Example 1). In previous studies both HPLC and mass spectrometry were used to confirm covalent biotinylation of peptides (Example 1).

Polyclonal Antibody

The following polyclonal antibodies to human histone H3 were generated using a commercial facility (Cocalico Biologicals, Reamstown, Pa.): anti-H3 (biotinylated at K4), anti-H3 (biotinylated at K9), and anti-H3 (biotinylated at K18). In order to raise these antibodies, the following peptides were custom-synthesized by the University of Virginia Biomolecular Research Facility: (i) N₁₋₁₃bioK4=ARTK(biotin)QTARKSTGGC (SEQ ID NO:33) (amino acids 1-13 in histone H3); (ii) N₁₋₁₃bioK9=ARTKQTARK(biotin)STGGC (SEQ ID NO:34) (amino acids 1-13); and (iii) N₁₃₋₂₅bioK18=GKAPRK(biotin)QLATKAAC (SEQ ID NO:35) (amino acids 13-25). Peptide identities were confirmed by mass spectrometry. Peptides were conjugated to keyhole limpet hemocyanin by utilizing the C-terminal cysteine (Example 1); these peptide conjugates were injected into white New Zealand rabbits. Booster injections were given after 14, 21, and 49 days. Serum was collected before immunization (pre-immune serum) and 2 days after each booster injection. Serum collected after the third booster injection was used for the assays described below; pre-immune serum was used as a control. For assessment of antibody specificities, electroblots of peptides N₁₋₁₃bioK4, N₁₋₁₃bioK9, and N₁₃₋₂₅bioK18 were probed with the anti-histone H3 antibodies and a monoclonal mouse anti-rabbit IgG peroxidase conjugate as described in Example 1; non-biotinylated peptide (N₁₋₂₅) was used as a control.

Immunocytochemistry

JAr human choriocarcinoma cells were cultured as described (Crisp et al., 2004). Biotinylated histones H3 in JAr human choriocarcinoma cells were visualized by standard procedures of immunohistochemistry (Cheung et al., 2003). Primary antibodies (serum) were diluted 250 fold. Pre-immune sera were used as negative controls. As secondary antibody we used Cy2-conjugated AffiniPure Donkey anti-Rabbit IgG (Jackson ImmunoResearch, West Grove, Pa.) at an 80-fold dilution. The nuclear compartment was stained using 4′,6-diamidino-2-phenylindole (DAPI), and the cytoplasm was stained using rhodamine phalloidin (Molecular Probes, Eugene, Oreg.). Images were obtained using Olympus FV500 confocal microscope equipped with an oil immersion lens.

Results

Biotinylation Sites in Histone H3

The N-terminal tail of histone H3 was efficiently biotinylated by biotinidase. The binding of biotin was substantially greater in peptide N₁₋₂₅ compared to peptide N₁₅₋₃₉, if equal amounts of both peptides were incubated with biotinidase and biocytin for 45 min (data not shown). The peptide (C₁₁₆₋₁₃₆) based on the C-terminus of histone H3 was not biotinylated if incubated with biotinidase (data not shown). This is consistent with previous observations that biotinylation and other modifications of histones cluster in the N-terminal region (Fischle et al., 2003; Example 1). Also these findings indicate that the primary targets for biotinylation are located in the region spanning the 25 N-terminal amino acids. Thus, subsequent studies focused on this region in the histone H3 molecule.

The studies in Example 1 above suggested that lysine residues in histones are targets for biotinylation. Thus, the inventors sub-divided the N-terminal 25 amino acids into four synthetic peptides to allow for easier identification of biotinylated lysines in histone H3: N₁₋₉ (including K4 and K9), N₉₋₁₆ (including K9 and K14), N₁₆₋₂₃ (including K18 and K23), and N₁₈₋₂₅ (including K18 and K23); subscripts denote the amino acid residues in the histone H3 sequence (amino acid sequence represented herein by SEQ ID NO:5). These peptides were incubated with biotinidase and biocytin for up to 45 min; at timed intervals aliquots were collected and biotinylated peptides on transblots were probed using streptavidin peroxidase. Peptide N₁₈₋₂₅ was a better substrate for biotinylation than peptides N₁₋₉, N₉₋₁₆, and N₁₆₋₂₃ (data not shown). Peptide N₁₋₂₅ was used as a reference and was heavily biotinylated (data not shown): 100% relative biotinylation after 45 min of incubation. Peptide C₁₁₆₋₁₃₆ was used as a negative control and was not biotinylated after 45 min. These results of this experiment indicated that either K18, K23, or both, are targets for biotinylation (see below). However, evidence is provided below that modifications of arginines may substantially enhance the biotinylation of histone H3 by biotinidase, and that K4 and K9 may also be targets for biotinylation in vivo. All subsequent enzymatic biotinylations were conducted for 45 minutes.

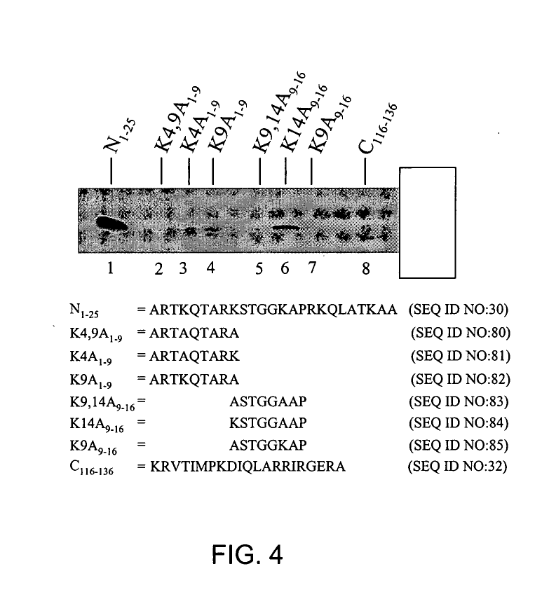

The next series of experiments focused on K4, K9, and K14. Peptide N₁₋₂₅ (SEQ ID NO:30) was used as a positive control and was heavily biotinylated (FIG. 4, lane 1). As expected, if both lysines (K4 and K9) in a peptide spanning amino acids 1 to 9 in histone H3 were substituted by alanine (K4,9A₁₋₉; SEQ ID NO:80), no binding of biotin was detectable (lane 2). This is consistent with the results of Example 1, indicating that lysines rather than other amino acids are targets for biotinylation. If K4 was substituted with alanine (K4A₁₋₉ SEQ ID NO:81), biotinylation of K9 was barely detectable (lane 3). In contrast, if K9 was substituted with alanine (K9A₁₋₉, SEQ ID NO:82), K4 was biotinylated considerably (lane 4). These findings indicate that K4 is a target for biotinylation.

Next, variations of a peptide spanning amino acids 9 to 16 in histone H3 (i.e., including K9 and K14) were tested. If both K9 and K14 were substituted with alanine (K9,14A₉₋₁₆; SEQ ID NO:83), no binding of biotin was detectable (lane 5). If K14 was substituted with alanine (K14A₉₋₁₆; SEQ ID NO:84), K9 was heavily biotinylated (lane 6). This is in contrast to the findings described above, which suggested that K9 is a poor target for biotinylation (peptide K4A₁₋₉ in lane 3). There is an explanation for these apparently contradictory observations: peptide K14A₉₋₁₆ is lacking the positively charged and bulky arginine residue in position 8; in contrast peptide K4A₁₋₉ includes R8. Biotinylation of K14A₉₋₁₆ can not be explained by biotinylation of K14, given that K14 is a poor target for biotinylation (peptide K9A₉₋₁₆, SEQ ID NO:85, lane 7). These findings are consistent with the hypothesis that K9 might be a good target for biotinylation if R8 is modified covalently; this hypothesis was further tested in dimethylation experiments described below. Peptide C₁₁₆₋₁₃₆ (SEQ ID NO:32) was used as a negative control; no biotinylation was detectable (lane 8).

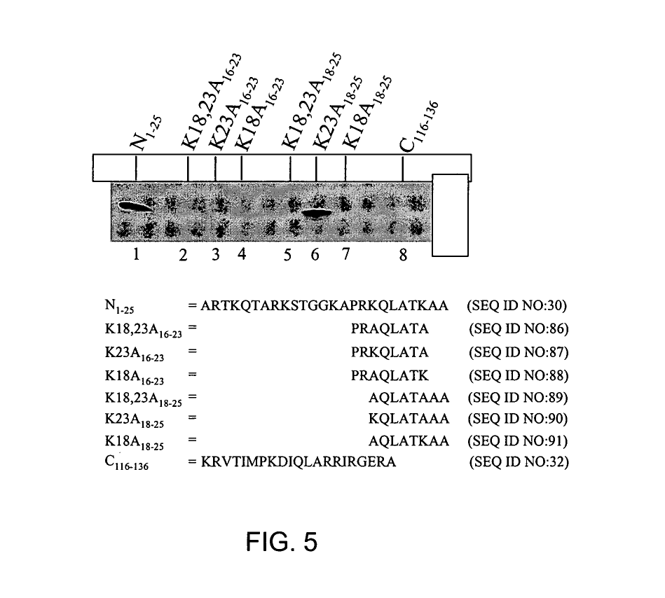

The following series of experiments focused on K18 and K23. Peptide N₁₋₂₅ (SEQ ID NO:30) was used as a positive control and was heavily biotinylated (FIG. 5, lane 1). As expected, if both lysines (K18 and K23) in a peptide based on amino acids 16 to 23 in histone H3 were substituted with alanine (peptide K18,23A₁₆₋₂₃; SEQ ID NO:86), no binding of biotin was detectable (lane 2). Likewise, biotinylation of K18 was weak if K23 was substituted with alanine (K23A₁₆₋₂₃; SEQ ID NO:87, lane 3), and biotinylation of K23 was weak if K18 was substituted with alanine (K18A₁₆₋₂₃; SEQ ID NO:88, lane 4). This is in apparent contrast to the findings discussed above, which suggested that K18 or K23 are good targets for biotinylation. Based on the following lines of reasoning, the inventors hypothesize that R17 in peptide K23A₁₆₋₂₃ interfered with biotinylation of K18 in the experiments depicted in FIG. 5: (i) Peptide N₁₈₋₂₅ starts with K18, i.e., does not include R17; (ii) peptide K23A₁₆₋₂₃ (FIG. 5) starts with A16, i.e., this peptide includes R17; (iii) experiments involving K9 suggested that arginine residues may interfere with biotinylation (see above). This hypothesis was tested as follows. Peptides were synthesized that started with K18 in histone H3; hence, these peptides did not include R17 but did include both K18 and K23 unless noted otherwise. No biotinylation was detected if both K18 and K23 were substituted with alanine (K18,23A₁₈₋₂₅; SEQ ID NO:89, lane 5). If K23 was substituted with alanine (K23A₁₈₋₂₅; SEQ ID NO:90), K18 was heavily biotinylated (lane 6). In contrast, if K18 was substituted with alanine (K₁₈A₁₈₋₂₅; SEQ ID NO:91), biotinylation of K23 was barely detectable (lane 7). Peptide C₁₁₆₋₁₃₆ (SEQ ID NO:32) was used as a negative control; no biotinylation was detectable (lane 8). These findings are consistent with the hypothesis that K18 is a target for biotinylation if R17 is modified; this hypothesis was further tested as described below. Also, these findings suggest that K23 is a poor target for biotinylation.

R2, R17, and many other arginine and lysine residues in human histones are modified by mono-, di-, and tri-methylation (Fischle et al., 2003; Lachner et al., 2003). Here the inventors determined whether naturally occurring modifications of arginines render lysines a better target for biotinylation in histone H3. Peptide N₁₆₋₂₃ was used as a control; this peptide includes K18 and K23, and an arginine residue (R₁₇) that is not di-methylated. Peptide N₁₆₋₂₃ was a moderate target for biotinylation by biotinidase (data not shown), confirming findings presented above. Likewise, peptides N₁₋₉ (including K4 and K9) and N₉₋₁₆ (including K9 and K14) were relatively poor targets for biotinylation (data not shown). Dimethylation of R2 and R8 (combined or individually) moderately increased the enzymatic biotinylation of K4 and K9 by biotinidase (data not shown). Dimethylation of R17 (peptide dmeR17₁₆₋₂₃) substantially increased the enzymatic biotinylation of K18 (data not shown). Note that peptide dmeR17₁₆₋₂₃ also contains K23; however, studies presented above suggested that K23 is a poor target for biotinylation.

Effects of arginine residues on biotinylation of lysines were further corroborated in the following series of experiments. The synthetic peptide N₆₋₁₃ (including R8 and K9) was used as a control; this peptide was a moderate target for biotinylation (Table 2). If R8 was substituted with an alanine (peptide R8A₆₋₁₃) biotinylation increased considerably, suggesting that unmodified arginines interfere with biotinylation of lysines by biotinidase. Substitution of arginine with omithine leaves intact the positive charge in position 8. If R8 was substituted with an ornithine (peptide R8O₆₋₁₃) biotinylation increased considerably, suggesting that the positive charge of arginine is not responsible for inhibiting biotinylation of lysines. If a negative charge was introduced by phosphorylation of S10 during peptide synthesis [S10S(p)₆₋₁₃], K9 became a poor target for biotinylation. This suggests that the naturally occurring phosphorylation of S10 (Fischle et al., 2003) may play a role in decreasing the availability of K9 for biotinylation. If K9 was substituted with an alanine (peptide K9A₆₋₁₃), no biotinylation was observed (negative control). Finally, changing the sequence of amino acids 7 and 8 from AR to RA did not substantially affect biotinylation of K9.

TABLE 2
Amino acid modifications affect biotinylation
of K9 by biotinidase^{a}

| | Amino acid | Relative | Sequence |
|---|---|---|---|
| Identifier | sequence | biotinylation | Identifier |
| N₆₋₁₃^{b} | TARKSTGG | ++ | SEQ ID NO: 36 |
| R8A₆₋₁₃ | TAAKSTGG | +++ | SEQ ID NO: 37 |
| R8O₆₋₁₃ | TAOKSTGG | +++ | SEQ ID NO: 38 |
| S10S(p)₆₋₁₃ | TARKS(p)TGG | − | SEQ ID NO: 39 |
| K9A₆₋₁₃ | TARASTGG | − | SEQ ID NO: 40 |
| AR7, 8RA₆₋₁₃ | TRAKSTGG | + | SEQ ID NO: 41 |

^{a}Peptides are denoted by using one-letter amino acid code.
^{b}TARKSTGG represents the native unmodified peptide, based on the amino acid sequence in position 6-13 in histone H3.

Polyclonal Antibody

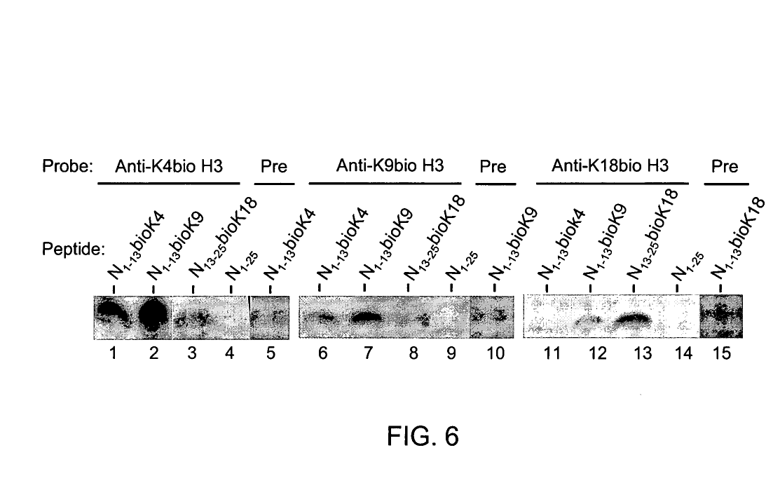

Polyclonal antibodies were generated to determine whether histone H3 is biotinylated at K4, K9, and K18 in vivo. First, the inventors determined whether the antibodies were specific for biotinylation sites. Transblots of the following biotinylated peptides were probed with the newly developed antibodies in all possible combinations: N₁₋₁₃bioK4, N₁₋₁₃bioK9, and N₁₃₋₂₅bioK18 (see Materials and Methods for sequence information). The following observations were made with regard to antibody specificities. The antibody raised against histone H3 (biotinylated at K4) reacted with N₁₋₁₃bioK4 and cross-reacted with N₁₋₁₃bioK9, but did not bind to N₁₃₋₂₅bioK18 (FIG. 6, lanes 1-3). No signal was detectable if non-biotinylated peptide (N₁₋₂₅) was used as a target (lane 4), or if N₁₋₁₃bioK4 was probed using pre-immune serum (lane 5). The antibody raised against histone H3 (biotinylated at K9) reacted with N₁₋₁₃bioK9, but cross-reacted only very weakly with N₁₋₁₃bioK4 and N₁₃₋₂₅bioK18 (lanes 6-8). No signal was detectable if non-biotinylated peptide (N₁₋₂₅) was used as a target (lane 9), or if N₁₋₁₃bioK9 was probed using pre-immune serum (lane 10). The antibody raised against histone H3 (biotinylated at K18) reacted with N₁₃₋₂₅bioK18, but did not bind to N₁₋₁₃bioK4 and cross-reacted only very weakly with N₁₋₁₃bioK9 (lanes 11-13). No signal was detectable if non-biotinylated peptide (N₁₋₂₅) was used as a target (lane 14), or if N₁₃₋₂₅bioK18 was probed using pre-immune serum (lane 15). Peptides N₁₋₁₃bioK4, N₁₋₁₃bioK9, and N₁₃₋₂₅bioK18 produced equal signals if biotin was probed with streptavidin-peroxidase (data not shown). This is consistent with the notion that equal amounts of peptide were loaded per lane in specificity experiments.

Finally, biotinylated species of histone H3 were visualized in JAr cells by using immunocytochemistry. Antibody to K4-biotinylated histone H3 localized primarily to the cell nucleus (data not shown); pre-immune serum did not generate a detectable signal. Likewise, staining with antibodies to K9-biotinylated and K18-biotinylated histone H3 was consistent with nuclear localization of biotinylated histones (data not shown). No signal was detectable if cells were stained with secondary antibody alone (data not shown). Staining with an antibody to K12-biotinylated histone H4 (see Example 1) also produced a nuclear signal (positive control; data not shown). Collectively, these findings indicate that human cells contain histone H3, biotinylated at K4, K9, and K18.

Discussion

This study provides evidence (i) that K4, K9, and K18 in histone H3 are good targets for biotinylation by human biotinidase; (ii) that K14 and K23 are relatively poor targets for biotinylation; (iii) that human cells contain histone H3, biotinylated in positions K4, K9, and K18; and (iv) that dimethylation of arginine residues in histone H3 enhances biotinylation of adjacent lysine residues, whereas phosphorylation of serine residues is likely to abolish biotinylation of adjacent lysine residues.

The following observations suggest that biotinylation of K4, K9, and K18 in histone H3 is physiologically important. First, evidence has been provided that biotinylation of histones might play a role in the cellular response to DNA damage (Peters et al., 2002; Kothapalli and Zempleni, 2004). Second, biotinylation of histones might be associated with gene silencing (Peters et al., 2002). Third, K4 and K9 are targets for both methylation (Fischle et al., 2003) and biotinylation; methylation and biotinylation of the same lysine residue are mutually exclusive. Methylation of K4 is associated with transcriptionally active chromatin whereas methylation of K9 is associated with transcriptionally silent chromatin (Jenuwein and Allis, 2001; Bird, 2001). Thus, biotinylation of K4 and K9 is likely to affect transcriptional activity of chromatin. Fourth, K18 is a target for both acetylation (Fischle et al., 2003; Lacher et al., 2003) and biotinylation. Acetylation of K18 is associated with transcriptionally active chromatin (Lachner et al., 2003). It is unknown whether biotinylation of K18 affects acetylation-dependent activation of chromatin.

Modifications of arginine residues in histones affect biotinylation of adjacent lysine residues. The following lines of evidence support this notion. (i) Dimethylation of R2, R8, and R17 increased biotinylation of K4, K9, and K18, respectively, by biotinidase. Dimethylation of R2 and R17 in histone H3 has been shown to occur in vivo (Fischle et al., 2003; Lacher et al., 2003), suggesting that the findings presented here are physiologically relevant. (ii) Substitution of R8 with ornithine was associated with increased biotinylation of K9. This is of potential physiological significance, given that monomethyl- and dimethyl-arginines in histones can be hydrolyzed to produce citrulline and, perhaps, ornithine (Bannister et al., 2002). Formally, the inventors cannot exclude the possibility that free amino groups in omithine and citrulline are substrates for biotinylation rather than enhancing biotinylation of adjacent lysines. However, the investigations of biotinylation motifs described herein suggested that ornithine is not biotinylated by biotinidase, and that citrulline is only a relatively poor target for biotinylation (data not shown).

Finally, the present study provides evidence that phosphorylation of serine residues may prevent biotinylation of adjacent lysine residues. This may be important for processes such as mitotic and meiotic chromosome condensation (phosphorylation of S10 and S28 in histone H3), transcriptional activation of chromatin (phosphorylation of S10 and S28 in histone H3), and DNA repair (phosphorylation of S14 in histone H2B) (Cheung et al., 2003; Lachner et al., 2003).

In the present study only biotinidase was used to identify biotinylation sites in histone H3. Theoretically, holocarboxylase synthetase might target distinct amino acid residues for biotinylation.

Taken together, the present study has revealed three new modifications of human histone H3: biotinylation of K4, K9, and K18. Previous studies suggested that K8 and K12 in histone The availability of site-specific antibodies to biotinylated histones described herein will generate novel insights into roles for histone biotinylation in eukaryotic cells.

### Example 3

The following example demonstrates the identification of residues that are biotinylated in histone 2A and antibodies that bind to such sites.

Materials and Methods

Identification of Biotinylation Sites

In Examples described above, the inventors developed a procedure to identify amino acid residues in histones that are targets for biotinylation. Briefly, this procedure is based on the following analytical sequence: (i) short peptides (<20 amino acids in length) are synthesized chemically; amino acid sequences in these peptides are based on the sequence in a given region of a histone; (ii) peptides are incubated with biotinidase or holocarboxylase synthetase (HCS) to conduct enzymatic biotinylation; and (iii) peptides are resolved by gel electrophoresis, and peptide-bound biotin is probed using streptavidin peroxidase. Amino acid substitutions (e.g., lysine-to-alanine substitutions) in synthetic peptides are used to corroborate identification of biotinylation sites. In addition, amino acid modifications (e.g., acetylation of lysines) in peptides can be used to investigate the cross-talk between biotinylation of histones and other known modifications of histones.

Here, peptides were synthesized based on the amino acid sequences in human H2A.1 (GenBank accession number M60752; amino acid sequence represented herein by SEQ ID NO:2) and H2A.X (GenBank accession number P16104; amino acid sequence represented herein by SEQ ID NO:3). Peptides were synthesized using N-fluoren-9-ylmethoxycarbonyl (Fmoc)-activated L-isomers of amino acids (see Example 1). One-letter annotation is used for denoting amino acids in this example (Garrett and Grisham, 1995). Chemically modified peptides were synthesized by using biotinylated, acetylated, and dimethylated ε-NH₂-derivatives of Fmoc-lysine, dimethylated guanidino derivatives of Fmoc-arginine, and phosphorylated derivatives of Fmoc-serine. Peptides were quantified as described in Example 1. Identities of peptides were confirmed by matrix assisted laser desorption ionization (MALDI)-time of flight and by quadrupole-time of flight mass spectrometry in the Nebraska Center of Mass Spectrometry (University of Nebraska-Lincoln). Amino acid sequences of synthetic peptides are provided below.

Peptides from both the N- and C-terminal regions of histone H2A and H2A.X were included in the analysis of biotinylation sites. Synthetic peptides were biotinylated enzymatically as described in Example 1 with the following modifications. Five micrograms of a given peptide were dissolved in 100 μL of a mixture containing 15 μL of human plasma (as a source of biotinidase), 10 μL of biocytin solution (75 μmol/L final concentration, as a source of biotin), and 75 μL of Tris buffer (50 mmol/L final concentration, pH 8.0). Samples were incubated at 37° C. for up to 45 minutes. Reactions were quenched by adding an equal volume of Tricine gel loading buffer (Invitrogen, Carlsbad, Calif.). Peptides were resolved by gel electrophoresis and peptide-bound biotin was probed by using streptavidin peroxidase (see Example 1).

Polyclonal Antibodies

The following biotinylation sites were identified in histone H2A in the experiments described below: K9 and K13 in the N-terminal region, and K125, K127, and K129 in the C-terminal region. Here, the inventors generated antibodies against K9-biotinylated histone H2A and K13-biotinylated histone H2A. In addition, the inventors generated antibodies against the two human enzymes that mediate biotinylation of histones: biotinidase and HCS. Polyclonal antibodies were produced using a commercial facility (Cocalico Biologicals, Reamstown, Pa.). The following peptides were synthesized by AnaSpec, Inc. (San Jose, Calif.) and the University of Virginia Biomolecular Research Facility (Charlottesville, Va.), respectively, for injection into rabbits: (i) N₁₋₁₂bioK9=SGRGKQGGK(biotin)ARAC (SEQ ID NO:42) (amino acids 1-12 in histone H2A plus a cysteine); (ii) N₁₀₋₂₄bioK13=ARAK(biotin)AKTRSSRAGLQC (SEQ ID NO:43) (amino acids 10-25 in histone H2A plus a cysteine); (iii) biotinidase (GenBank accession number NM_{—}000060; amino acid sequence represented herein by SEQ ID NO:44)=CLRKSRLSSGLVTAALYGRLYERD (SEQ ID NO:45) (amino acids 520-542 in biotinidase plus one cysteine); and (iv) HCS (GenBank accession number NM_{—}000411; amino acid sequence represented herein by SEQ ID NO:46)=EHVGRDDPKALGEEPKQRRGC (SEQ ID NO:47) (amino acids 58-77 in HCS plus one cysteine). Identities and purities of these peptides were confirmed by using high-performance liquid chromatography (HPLC) and MALDI (data not shown). Peptides were conjugated to keyhole limpet hemocyanin before injection into White New Zealand rabbits as described in Example 1. Rabbit serum was collected before (pre-immune serum) and after three injections with peptides mixed with Freund's adjuvant over a period of 49 days. Immunoglobulin G was purified from serum by using the ImmunoPure (A) IgG Purification Kit (Pierce, Rockford, Ill.) according to the manufacturer's protocol. Antibody specificities were investigated by using synthetic peptides and histone extracts from human cells as described in Example 1.

Cell Culture

Human-derived Jurkat lymphoma cells and JAr choriocarcinoma cells (ATCC, Manassas, Va.) were cultured as described (Manthey et al., 2002; Crisp et al., 2004). Acid extracts from Jurkat cell nuclei (Stanley et al., 2001) were used for western blot analysis of biotinylated histone H2A, as described for H4 in Example 1, whereas JAr cells were used for analysis of biotinylated histone H2A by immunocytochemistry.

Immunocytochemistry

K9-biotinylated histone H2A, K13-biotinylated histone H2A, biotinidase, and HCS were visualized in JAr cells by using immunocytochemistry as described (Cheung et al., 2003). Primary antibodies were as described above. As a secondary antibody the inventors used donkey anti-rabbit Cy2-labeled antibody (Jackson ImmunoResearch, West Grove, Pa.). Cytoplasmic and nuclear compartments were stained with rhodamine phalloidin and 4′, 6-diamidino-2-phenylindole (DAPI) (Sigma, St. Louis, Mo.) as described (Cheung et al., 2003). Images were obtained by using an Olympus FV500 confocal microscope (Microscopy Core Facility, University of Nebraska-Lincoln).

Results

Biotinylation Sites in Histones H2A and H2A.X

Both the N- and C-termini of histone H2A contain targets for biotinylation by biotinidase. The inventors synthesized the following five peptides based on the N- and C-termini of histone H2A: N₁₋₉=amino acid sequence SGRGKQGGK (SEQ ID NO:48); N₇₋₁₄=GGKARAKA (SEQ ID NO:49); N₁₂₋₂₀=AKAKTRSSR (SEQ ID NO:50); C₁₁₃₋₁₂₁=AVLLPKKTE (SEQ ID NO:51); and C₁₂₂₋₁₂₉=SHHKAKGK (SEQ ID NO:52); subscript numbers denote the position of amino acid residues in histone H2A. These peptides were subjected to enzymatic biotinylation, and peptide-bound biotin was probed using gel electrophoresis and streptavidin peroxidase. Both N₁₋₉ and N₇₋₁₄ were good targets for biotinylation by biotinidase but N₁₂₋₂₀ was not a good target (data not shown). Moreover, peptide C₁₁₃₋₁₂₁ was not good target, but the C-terminal C₁₂₂₋₁₂₉ was a good target for biotinylation. These results are consistent with the results in Examples 1 and 2 showing that lysine residues in these peptides are the most likely targets for biotinylation.

The inventors verified that peptide biotinylation approached maximal levels under the conditions described in Methods and Materials. First, the time course of biotinylation of peptide N₁₋₉ was monitored at timed intervals for up to 45 minutes; concentrations of peptide, biocytin, and biotinidase were kept constant as described above. Biotinylation of peptide N₁₋₉ was detectable 15 minutes after starting the incubation with biotinidase and reached maximal levels after 45 minutes (data not shown). Second, the inventors tested effects of substrate (biocytin) availability. Peptide N₁₋₉ was incubated with biotinidase at various concentrations of biocytin (7.5, 37.5, 75, 112.5, and 150 μmol/L) for 45 minutes. Biotinylation of N₁₋₉ reached a plateau at 75 μmol/L of biocytin (data not shown). Finally, the inventors varied the concentration of peptide N₁₋₉ in the biotinylation reaction. The biotinylation signal paralleled the amount of N₁₋₉ added to incubation mixtures (data not shown).

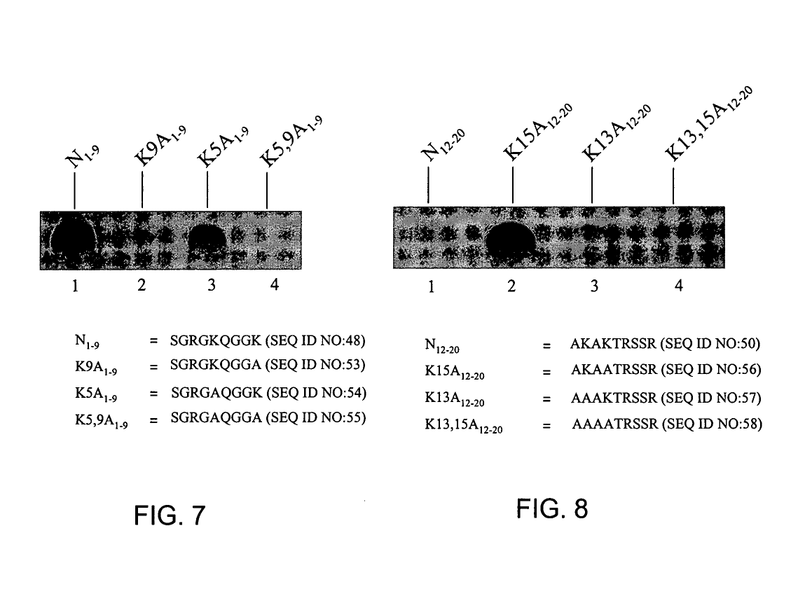

Next, biotinylation targets were identified in the N-terminus of histone H2A. A first series of experiments suggested that K9 is a biotinylation target, based on the following lines of evidence. Peptide N₁₋₉ (SEQ ID NO:48; containing both K5 and K9) was heavily biotinylated in response to incubation with biotinidase (FIG. 7, lane 1). If K9 was substituted with alanine (peptide K9A₁₋₉; SEQ ID NO:53) no biotinylation was detectable (lane 2). In contrast, substitution of K5 with alanine residues (peptide K5A₁₋₉; SEQ ID NO:54) did not decrease biotinylation (lane 3). If both lysine residues in peptide N₁₋₉ were substituted with alanines (peptide K5,9A₁₋₉; SEQ ID NO:55) no biotinylation was detectable (lane 4). Biotinylation of K9 by biotinidase was further corroborated using the following control. Peptide N₇₋₁₄ contains both K9 and K13 from histone H2A, and was heavily biotinylated in response to incubation with biocytin and biotinidase (data not shown). If K13 in N₇₋₁₄ was substituted with alanine, the biotinylation signal decreased only moderately; in contrast, if K9 was substituted with an alanine the biotinylation signal decreased substantially (data not shown).

A second series of experiments suggested that K13 in the N-terminus of histone H2A becomes a target for biotinylation if the neighboring K15 is modified. This notion is based on the following lines of evidence. Peptide N₁₂₋₂₀ (SEQ ID NO:50) contains both K13 and K15 and was a poor target for biotinylation by biotinidase (FIG. 8, lane 1). However, if K15 was substituted with an alanine (peptide K15A₁₂₋₂₀; SEQ ID NO:56), K13 became a good target for biotinylation (lane 2). Substitution of K13 with an alanine (peptide K13A₁₂₋₂₀; SEQ ID NO:57) did not render K15 a good target for biotinylation (lane 3). If both lysine residues in peptide N₁₂₋₂₀ were substituted with alanine residues (peptide K13,15A₁₂₋₂₀; SEQ ID NO:58) no biotinylation was detectable (lane 4). Note that the lysine-to-alanine substitutions used here are an artificial system that does not necessarily represent histones from human cells. However, the findings described below suggest that naturally occurring variations in amino acid sequences (see histone variant H2A.X) and posttranslational modifications of amino acids (see cross-talk among histone modifications) render K13 a good target for biotinylation.

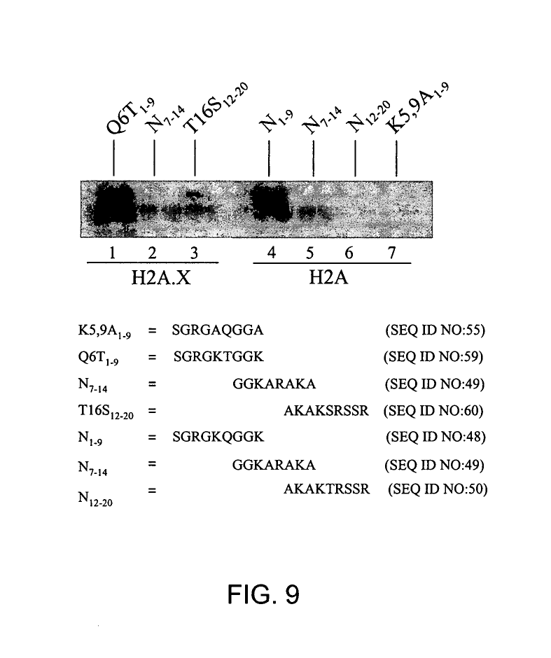

The N-terminal tail of human histone H2A.X differs from the tail in histone H2A in two positions (Wyatt et al., 2003): glutamine in position 6 is substituted with threonine, and threonine in position 16 is substituted with serine in histone H2A.X. First, the inventors synthesized the following two peptides based on the N-terminus of histone H2A.X: Q6T₁₋₉=amino acid sequence SGRGKTGGK (SEQ ID NO:59), and T16S₁₂₋₂₀=AKAKSRSSR (SEQ ID NO:60). Both peptides were good targets for biotinylation by biotinidase (FIG. 9, lanes 1 and 3). Peptide N₇₋₁₄ represents a moderate target for biotinylation (see above) and was used as a control (lanes 2 and 5). In fact, the N-terminus of histone H2A.X was a better target for biotinylation than the N-terminus of histone H2A (compare lanes 1-3 with lanes 4-6). Specifically, the peptide containing both K9 and K13 (Q6T₁₋₉) was a good target for biotinylation (lane 1), whereas the peptide containing K13 and K15 (T16S₁₂₋₂₀) was biotinylated only moderately in response to incubation with biotinidase (lane 3). Peptide K5,9A₁₋₉ does not contain any lysine residues and was used as a negative control (lane 7); no biotinylation was detectable after incubation with biotinidase.

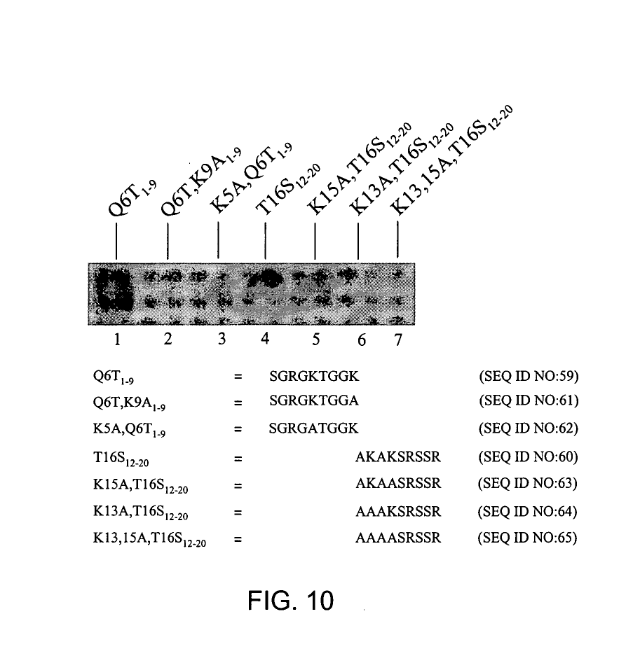

In a next series of experiments, the inventors confirmed that K9 and K13 in variant H2A.X are specifically targeted by biotinylation in analogy to the findings described for histone H2A. Overall, the same trends were observed for peptides based on histone H2A.X compared with histone H2A. Peptide Q6T₁₋₉ (SEQ ID NO:59) contains both K9 and K13 from histone H2A.X and was biotinylated in response to incubation with biotinidase (FIG. 10, lane 1). Substitution of K9 with alanine (peptide Q6T,K9A₁₋₉; SEQ ID NO:61) substantially decreased biotinylation (lane 2), whereas substitution of K5 with alanine (peptide Q6T,K5A₁₋₉; SEQ ID NO:62) decreased biotinylation only moderately (lane 3). Peptide T16S₁₂₋₂₀ (SEQ ID NO:60) contains both K13 and K15 and was biotinylated in response to incubation with biotinidase (lane 4). If K15 was substituted with alanine (peptide K15A,T16S₁₂₋₂₀; SEQ ID NO:63) biotinylation decreased moderately (lane 5). No biotinylation was detectable if K13 was substituted with alanine (peptide K13A,T16S₁₂₋₂₀; SEQ ID NO:64, lane 6). If both lysine residues in peptide T16S₁₂₋₂₀ were substituted with alanine (peptide K13,15A,T16S₁₂₋₂₀; SEQ ID NO:65) no biotinylation was detectable (lane 7).

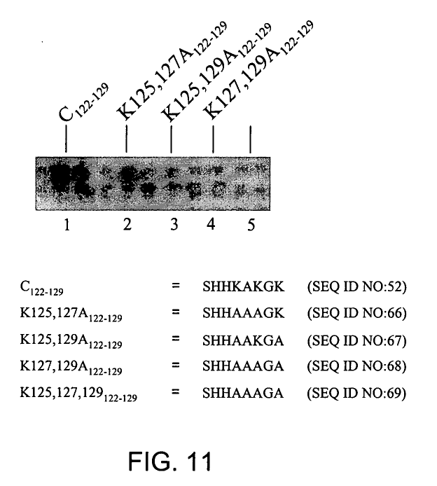

Lysines in the C-terminus of histone H2A were targeted for biotinylation by biotinidase. The C-terminus of histone H2A contains three lysine residues in positions 125, 127, and 129. A synthetic peptide including all three of these lysines (C₁₂₂₋₁₂₉) was a good substrate for biotinylation by biotinidase (FIG. 11, lane 1). Biotinylation decreased only moderately, if K125 and K127 were substituted with alanine residues (peptide K125,127A₁₂₂₋₁₂₉; SEQ ID NO:66, lane 2), suggesting that K129 is a good target for biotinylation. Consistent with this hypothesis, substitution of K125 and K129 (peptide K125,129A₁₂₂₋₁₂₉; SEQ ID NO:67), and K127 and K129 (peptide K127,129A₁₂₂₋₁₂₉; SEQ ID NO:68) with alanine residues caused a considerable decrease of biotinylation (lanes 3 and 4, respectively). If all lysine residues in peptide C₁₂₂₋₁₂₉ were substituted with alanine residues (peptide K125,127,129A₁₂₂₋₁₂₉; SEQ ID NO:69) no biotinylation was detectable (lane 5).

The C-terminus of histone H2A.X was not a good target for biotinylation. Note, that N-terminal sequences are highly conserved between histones H2A and H2A.X, but that the C-terminal sequences of these two histones are unique (Wyatt et al., 2003). Here, the inventors synthesized the following three peptides based on the C-terminus of histone H2A.X: C₁₁₃₋₁₂₁=AVLLPKKTS (SEQ ID NO:70), C₁₂₂₋₁₃₁=ATVGPKAPSG (SEQ ID NO:71), and C₁₃₂₋₁₄₂=GKKATQASQEY (SEQ ID NO:72). These peptides were not biotinylated in response to incubation with biotinidase (data not shown).

Cross-talk Among Histone Modifications

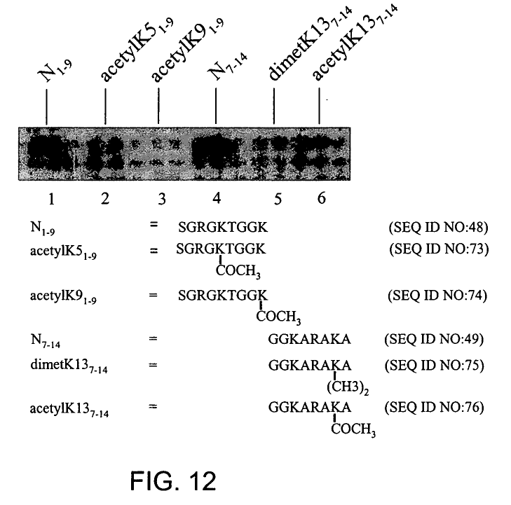

The studies in Example 1 indicated that acetylation and methylation of histone H4 affect subsequent biotinylation. Of note, K5, K9, K13, and other lysine residues in human histone H2A are targets for acetylation (Zhang et al., 2003). Here, the inventors provide evidence that acetylation and methylation of histone H2A are likely to affect subsequent biotinylation. Peptide N₁₋₉ (SEQ ID NO:48, containing both K5 and K9) was heavily biotinylated in response to incubation with biotinidase, and was used as a positive control (FIG. 12, lane 1). Acetylation of K5 caused a moderate decrease in the decreased the biotinylation of K9 (SEQ ID NO:73, lane 2). A peptide containing acetylated K9 and free K5 was not a target for biotinylation (SEQ ID NO:74, lane 3). This is consistent with the observation that K9 but not K5 is a target for biotinylation (see above). Moreover, dimethylation of R3 did not cause a change in the biotinylation signal (data not shown), because the adjacent K5 is not a biotinylation target. Peptide N₇₋₁₄ (SEQ ID NO:49, containing both K9 and K13) was a good target for biotinylation (lane 4). Again, acetylation of K9 decreased the biotinylation signal substantially (data not shown). Moreover, both dimethylation and acetylation of K13 decreased the biotinylation of K9 (SEQ ID NO:75, lane 5 and SEQ ID NO:76, lane 6). On the other hand, dimethylation R11 considerably increased the enzymatic biotinylation of K9 or K13 (or both) by biotinidase (data not shown). Peptide N₁₂₋₂₀ (containing both K13 and K15) was a poor target for biotinylation, but dimethylation of R17 substantially increased the biotinylation of K13 or K15 (or both) (data not shown).

Biotinylation of Histone H2A in Human Cells

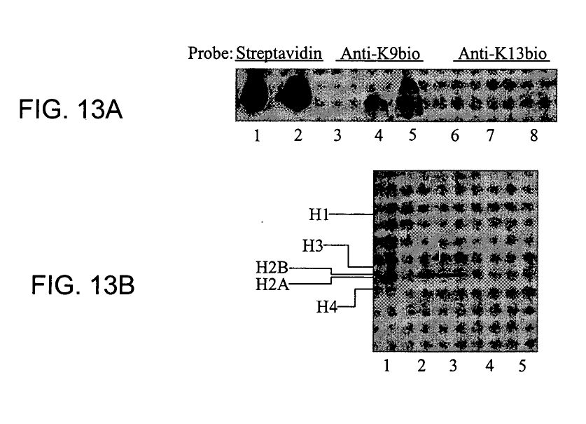

Human cells contain biotinylated histone H2A, as judged by using novel biotinylation site-specific antibodies. In a first series of experiments the inventors raised antibodies to K9-biotinylated and K13-biotinylated histone H2A, and validated the specificity of these antibodies by using synthetic peptides. The same two peptides that were used for injections into rabbits (N₁₋₁₂bioK9 and N₁₀₋₂₅bioK13) were run on a polyacrylamide gel, and the biotin tag was probed by using streptavidin peroxidase. The two peptides produced a similar signal (FIG. 13A, compare lane 1 and 2), suggesting that biotinylation of peptides and loading of peptides on gels was similar. Second, the two peptides and a non-biotinylated control (N₁₋₂₀) were probed with antibodies to K9- and K13-biotinylated histone H2A. Anti-K9bio antibody did not bind to peptide N₁₋₂₀ (lanes 3) but cross-reacted with both biotinylated peptides: N₁₀₋₂₅bioK9 and N₁₀₋₂₅bioK13 (lanes 4 and 5); pre-immune serum did not produce a detectable signal (data not shown). In contrast, anti-K13bio antibody did not bind to N₁₋₂₀ and peptide N₁₀₋₂₅bioK9 (lanes 6 and 7), but was specific for peptide N₁₀₋₂₅bioK13 containing biotinylated K13 (lane 8); pre-immune serum did not produce a detectable signal (data not shown). Moreover, antibodies to biotinylated histone H2A did not cross-reacted with biotinylated peptides based on histone H4: N₆₋₁₅bioK8=GGK(biotin)GLGKGGA (SEQ ID NO:77) and N₆₋₁₅bioK12=GGKGLGK(biotin)GGA (SEQ ID NO:78) (data not shown). Collectively, these data suggest that both anti-K9bio and anti-K13bio are specific for biotinylated histone H2A peptides and are unlikely to cross-react with other biotinylated histones (see below). These data also suggest that antibody anti-K13bio is biotinylation site-specific, whereas antibody anti-K9bio cross-reacts with both biotinylated K9 and K13.

Next, histone extracts from Jurkat cell nuclei were probed with antibodies to biotinylated histone H2A. The histone extracts contained biotinylated histones H1, H2A, H2B, H3 and H4, as judged by staining with streptavidin peroxidase (FIG. 13B, lane 1). The polyclonal antibodies raised in this study were specific for histone H2A and did not cross-react with other classes of histones (lanes 2 and 3). If biotinylated histones were probed with pre-immune serum, no detectable signal was produced (lanes 4 and 5).

Biotinylated histone H2A localized to the nucleus in JAr choriocarcinoma cells, as judged by confocal microscopy and antibodies against biotinylated histone H2A. First, the subcellular localization of K9-biotinylated histone H2A was visualized using antibody anti-K9bio (data not shown). Nuclear and cytoplasmic compartment were stained with DAPI and rhodamine phalloidin, respectively. Merged images are consistent with nuclear localization of K9-biotinylated histone H2A. Pre-immune serum did not generate a detectable signal. Analogous experiments were conducted for K13-biotinylated histone H2A. Antibody anti-K13bio localized primarily to the cell nucleus (data not shown).

Both biotinidase and HCS showed considerable nuclear localization in JAr cells. First, we validated the specificity of antibodies against biotinidase and HCS using synthetic peptides as described for histone antibodies (data not shown). The subcellular localization of biotinidase in JAr cells was visualized using confocal microscopy and anti-biotinidase (data not shown). Nuclear and cytoplasmic compartment were stained with DAPI and rhodamine phalloidin, respectively. Merged images were consistent with nuclear localization of biotinidase. Pre-immune serum did not generate a detectable signal. Analogous experiments were conducted for HCS. Anti-HCS also localized to the cell nucleus; pre-immune serum (negative control) did not generate a detectable signal (data not shown). These findings are consistent with a role for biotinidase and HCS in chromatin structure, mediated by biotinylation of histones.

Discussion

This study provides evidence that (a) K9 and K13 in the N-terminus of histones H2A and H2A.X are targets for biotinylation by biotinidase; (b) that K125, K127, and K129 in the C-terminus of histone H2A are targets for biotinylation by biotinidase; (c) that K9- and K13-biotinylated histone H2A reside in human cell nuclei; (d) that acetylation and dimethylation of lysine residues in histones decrease subsequent biotinylation of adjacent lysine residues; (e) that dimethylation of arginine residues increases subsequent biotinylation of adjacent lysine residues; and (f) that both HCS and biotinidase reside primarily in the nuclear compartment.

The following observations suggest that biotinylation of histone H2A is physiologically important. First, biotinylation of histones plays a role in the regulation of gene expression (Peters et al., 2002), cell proliferation (Stanley et al., 2001; Narang et al., 2004), and cellular response to DNA damage (Peters et al., 2002; Kothapalli et al., 2004). Second, acetylation of K9 in histone H2A might be associated with transcriptionally active chromatin (Turner, 2002). The present study suggests that K9 is targeted by two mutually exclusive modifications: acetylation and biotinylation. Without being bound by theory, the present inventors believe that biotinylation of K9 affects the transcriptional activity of chromatin. Third, phosphorylation of histone H2A.X is known to participate in DNA repair events, mediated by accumulation at sites of DNA damage (Paull et al., 2000). Biotinylation of histones is known to change in response to DNA damage (Peters et al., 2002; Kothapalli et al., 2004), but it remains to be determined whether biotinylation of K9 and K13 in histone H2A plays a role in repair events. Fourth, biotinylation of lysines in the C-terminus of histone H2A might affect histone-histone interactions in nucleosomes, based on the following lines of reasoning. Histone H2A is unique among core histones in having its C-terminal tail exposed at the nucleosomal surface (Wolffe, 1998; Luger et al., 1997). However, the larger part of the C-terminal domain of histone H2A and other histones is buried inside the nucleosomes (Wolffe, 1998). The C-terminal histone fold domain is predominantly α-helical with a long central helix bordered on each side by a loop segment (β-bridge, hinge region) and a shorter helix (Wolffe, 1998). The long helix acts as a dimerization interface between histones (Wolffe, 1998). Without being bound by theory, the present inventors believe that the biotinylation of C-terminal lysine residues in histone H2A affects the dimerization of histones. Note that K125 or K127 are also targets for methylation (Zhang et al., 2003). Effects of lysine methylation in the C-terminus of histone H2A are uncertain, but interactions between biotinylation and methylation are likely to occur in vivo.

The results in Example 1 suggested that dimethylation of arginine residues in histone H4 increases biotinylation of adjacent lysine residues. The inventors observed a similar pattern for histone H2A: dimethylation of R11 increased the biotinylation of K9 or K13 (or both) by biotinidase, and dimethylation of R17 increased the biotinylation of K13 or K15 (or both). Note that arginine residues in histones can be converted to citrulline and omithine by deimination (Bannister et al., 2002; Cuthbert et al., 2004). Citrulline and ornithine residues are good targets for biotinylation by biotinidase (data not shown). Collectively, posttranslational modifications of arginine residues are likely to play important roles in histone biotinylation.

Finally, this study provides evidence that significant fractions of cellular biotinidase and HCS localize to the nuclear compartment. Previous studies are consistent with a nuclear localization of HCS (Narang et al., 2004). Narang et al. suggested that the majority of HCS localized to the nuclear periphery rather than the nucleoplasm. The functional significance of this observation is currently being investigated. The amino acid residues in histones that are targets for biotinylation by HCS await identification, whereas targets for biotinidase have been characterized (Example 1 and Example 2). Unlike for HCS, the cellular distribution of biotinidase is controversial. This study and a previous study (Pispa, 1965) are consistent with nuclear localization of biotinidase; in contrast, Wolf and co-workers suggested that biotinidase localizes to the cytoplasm but not to the nucleus (Stanley et al., 2004). The reasons for these apparently conflicting observations are unknown.

### Example 4

The following example describes an avidin-based assay to quantify histone debiotinylase activities in nuclear extracts from eukaryotic cells and the use of such assay to (i) to quantify histone debiotinylase activities in nuclei from various human tissues; and (ii) to determine whether histone debiotinylase activity depends on the cell cycle.

Materials and Methods

Cell Culture

The following cell lines were obtained from American Type Culture Collection (Manassas, Va.): HepG2 hepatocarcinoma cells, JAr choriocarcinoma cells, Jurkat cells (clone E6-1), HCT-116 colone cancer cells, and NCI-H69 small cell lung cancer cells. Cells were cultured in humidified atmosphere (5% CO2 at 37° C.) as described (Rodriguez-Melendez et al., 2005; Manthey et al., 2002; Scheerger and Zempleni, 2003; Crisp et al., 2004). Cell viability was monitored periodically using the Trypan blue exclusion test (Zempleni and Mock, 1998). For cell cycle studies, NCI-H69 cells were treated with 2 mM thymidine for 48 h (G1 phase arrest), 2 mM hydroxyurea for 40 h (S phase arrest), and 10 μM etoposide for 40 h (G2 phase arrest) (Chaudhry et al., 2002; Van Hooser et al., 1998; Allison et al., 2003). M phase arrest was achieved by the following sequential treatment of cells: 2 mM thymidine for 18 h; culturing without thymidine for 3 h; and 0.33 μM nocodazole for 12 h (Whitfield et al., 2000). Cell cycle arrest was confirmed using propidium iodide-stained cells and flow cytometry (Vindelov, 1977) in the Flow Analysis Core Facility of the University of Nebraska Medical Center (Omaha, Nebr.).

Protein Extracts

Proteins were extracted from cell nuclei and cytoplasm by using the Nuclear Extract Kit (Active Motif, Carlsbad, Calif.) according to the manufacturer's instructions. Protein concentrations in extracts were determined using the bicinchoninic acid method (Pierce, Rockford, Ill.). Protein concentrations were adjusted as needed by dilution with water.

Biotin Debiotinylation Assay

Calf thymus histone H1 (Calbiochem, San Diego, Calif.) was biotinylated using biotinidase to produce substrate for histone debiotinylases in subsequent assays. Briefly, 1 mg of histone H1 was dissolved in a mixture of 0.6 ml of human plasma (as a source of biotinidase), 0.4 ml of 750 μM biocytin (biotinyl-ε-lysine, as a source of biotin), and 19 ml of 50 mM Tris buffer [pH 8.0]; the mixture was incubated at 37° C. for 45 min in a waterbath (see Example 1). In Example 1, the inventors confirmed covalent binding of biotin to histones by using HPLC and mass spectrometry. Thirty milliliters of 50 mM carbonate buffer [pH 9.6] were added to the histone solution, and 100 μl of the mixture were dispensed into 96-well plates for overnight coating at 4° C. Coating efficiency depended substantially on the brand of plate used. The best results were obtained using Falcon Microtest plates (Becton Dickinson, Franklin Lakes, N.J.), although other brands may be sufficient. Next, the histone-containing buffer was discarded and wells were blocked using 200 μl of 0.1% bovine serum albumin [w/v] and 0.05% Tween-20 [v/v] in phosphate-buffered saline (PBS) [pH 7.4] at 4° C. for at least 4 h. For histone debiotinylation assays, plates were washed twice using 250 μl of PBS. Fifty microliters of cellular protein extract (typically containing 20 μg of protein) were mixed with 100 μl of 50 mM Tris buffer [pH 7.4], and were transferred into microwell plates to initiate enzymatic debiotinylation of histones adhered to plastic surfaces; incubation times and temperatures were as provided in Results. Typically, protein-free Tris buffer was used as a negative control but other controls were also tested (see below). Debiotinylation was terminated by washing the plates twice with 200 μl of PBS. Histone-bound biotin remaining in the plates was probed using 100 μl of avidin-conjugated horseradish peroxidase (10 μg/l in a buffer containing 0.1% BSA [w/v] in PBS) at room temperature for 1 h. Plates were washed twice using 0.05% Tween-20 in PBS [w/v]. Immobilized horseradish peroxidase was visualized using 100 μl of SureBlue TMB [3,3′,5,5′-tetra-methylbenzidine] Microwell Peroxidase Substrate (KPL, Inc.; Gaithersburg, Md.) at room temperature for 30 min; the reaction was terminated by adding 100 μl of TMB Stop Solution (KPL, Inc.; Gaithersburg, Md.). The absorbance was read at 450 nm in an Emax Microplate reader (Molecular Devices, Sunnyvale, Calif.). Note that a low absorbance at 450 nm is consistent with a great histone debiotinylase activity in biological samples. A calibration curve was generated by incubating a dilution series of avidin-conjugated horseradish peroxidase (up to 1.4 fmol/well) in uncoated plates with 100 μl of SureBlue TMB Microwell Peroxidase Substrate. Calibration was based on the assumption that on average one molecule of avidin is conjugated to two molecules of horseradish peroxidase, producing a molecular weight of 147 kDa.

Proteolytic Digestion of Histone H1

Here the inventors determined whether biotin release is mediated by proteolytic digestion of histones. One milligram of histone H1 was dissolved in 100 μl of 20 mM sodium acetate [pH 4.5]. 0.4 microliters of histone solution was mixed with 7.1 μl of nuclear extract containing 20 μg of protein; samples were incubated at 37° C. for 20 min. The following controls were tested: histones incubated without nuclear extract, and histones incubated with 2.5 microliter trypsin [with or without 20 mM of the trypsin inhibitor, phenylmethylsulphonylfluoride (PMSF)]. Reactions were terminated by heating the samples with equal volume of loading buffer at 72° C. for 10 min. Proteins were resolved using 4-12% Bis-Tris gels (Invitrogen, Carlsbad, Calif.) as described (Stanley et al., 2001) and were visualized using coomassie blue.

Antibody to Human Biotinidase

A peptide based on amino acids 520 to 542 (amino acid sequence=LRKSRLSSGLVTAALYGRLYERD; SEQ ID NO:79) in human biotinidase (GenBank NM_{—}000060; amino acid sequence represented herein by SEQ ID NO:44) was purchased from the University of Virginia Biomolecular Research Facility (Charlotteville, Va.); identity and purity of the peptide were confirmed by mass spectrometry and HPLC. The peptide was conjugated to keyhole limpet cyanine using an N-terminal cysteine residue, and polyclonal antibodies to human biotinidase were raised in rabbits using a commercial facility (Cocalico, Inc. Reamstown, Pa.) as described in Example 1. Antibody specificity was validated extensively by using synthetic peptides, recombinant biotinidase, and human plasma as described in Example 1. Pre-immune serum was used as a negative control.

Immunocytochemistry

The cellular distribution of biotinidase was visualized by using standard procedures of immunocytochemistry (Cheung et al., 2003). JAr cells were stained with rabbit anti-human biotinidase antibody and Cy™2-conjugated donkey anti-rabbit IgG (Jackson ImmunoResearch, West Grove, Pa.). Cytoplasmic compartment was stained with rhodamine phalloidin (Molecular Probes, Eugene, Oreg.). 4′,6′-Diamidino-2-phenylindole (DAPI) was used to stain DNA in the nucleus. Cells were viewed with an Olympus FV500 confocal microscope equipped with a 40× oil immersion lens (Microscopy Core Facility, University of Nebraska-Lincoln).

Statistics

Homogeneity of variances among groups was confirmed using Bartlett's test (SAS Institute Inc., 1999). Significance of differences among groups was tested by one-way ANOVA. Fisher's Protected Least Significant Difference procedure was used for posthoc testing (SAS Institute Inc., 1999). Student's paired t-test was used for pairwise comparisons. StatView 5.0.1 (SAS Institute; Cary, N.C.) was used to perform all calculations. Differences were considered significant if P<0.05. Data are expressed as mean±SD.

Results

Calibration and Linearity of the Histone Debiotinylase Assay

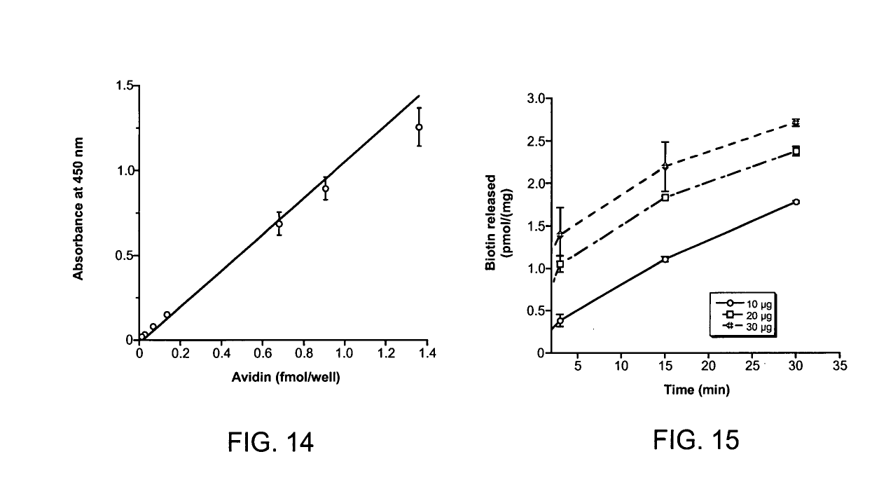

First, TMB substrate was mixed with avidin-horseradish peroxidase in uncoated 96-well plates to identify the linear range of the detection system. The apparent oxidation of TMB increased linearly up to 0.7 fmoles of avidin-horseradish peroxidase per well, as judged by the absorbance at 450 nm (FIG. 14). Subsequent histone debiotinylation assays were calibrated using avidin standards from within the linear range. For purposes of calibration it was assumed that one molecule of avidin binds four molecules of biotin (Green, 1975). This might slightly overestimate the amount of biotin released by histone debiotinylases, given that not all biotin-binding sites in avidin might participate in biotin binding due to spatial effects (Green, 1990).

Incubation of 96-well plates with nuclear extracts from NCI-H69 cells caused a time- and protein-dependent release of biotin from histone H1. This is consistent with the presence of histone debiotinylases in human cell nuclei. The release of biotin was linear for up to at least 5 μg of protein added per well (FIG. 15). The assays described below were conducted using 2.5 μg of nuclear protein per well and an incubation time of 15 min.

Temperature and pH

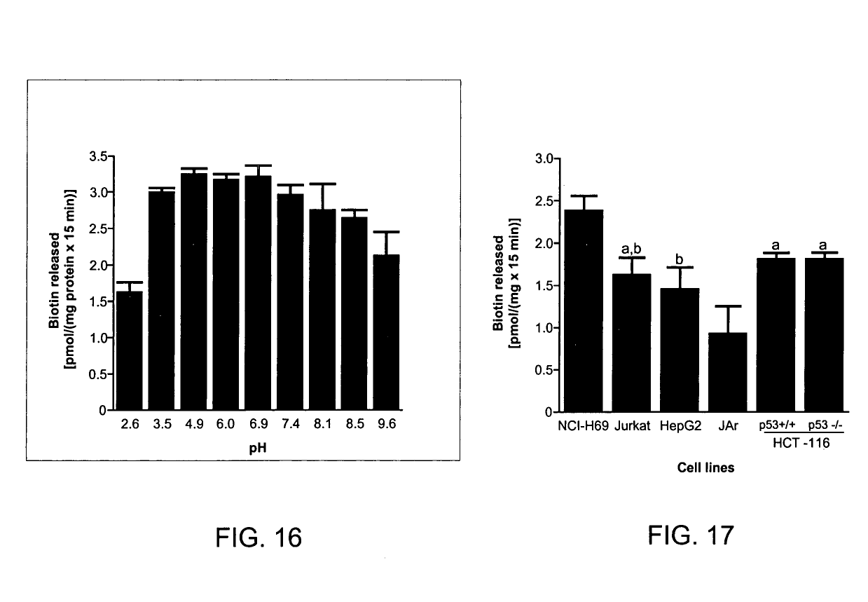

The debiotinylation of histone H1 by nuclear extracts from NCI-H69 cells was temperature dependent [units=pmol biotin released/(mg protein×15 min)]: 1.8±0.02 at 37° C. and no debiotinylation at 22° C. (data not shown). Moreover, the rate of histone debiotinylation depended on the pH of the incubation buffer (FIG. 16). The pH optimum of putative histone debiotinylases was rather broad and spanned the range from pH 4 to 8; all subsequent experiments were conducted at pH 7.4 and 3.7° C. Both temperature and pH dependence of histone debiotinylation are consistent with an enzyme-mediated process. Consistent with this notion, histone debiotinylase activity was destroyed if nuclear extracts were boiled before incubation of plates. Finally, no debiotinylation of histone H1 was detectable if plates were incubated with protein-free nuclear extraction buffer (data not shown). This is consistent with the hypothesis that release of biotin was not caused by physical desorption of histones from plastic surfaces.

Proteolysis

Release of biotin from histones was mediated by debiotinylases rather than by proteolytic degradation of plate-bound histone H1. This notion is based on the following lines of evidence. If histone H1 was incubated with nuclear extract from NCI-H69 cells, no degradation of histone was detectable by gel electrophoresis (data not shown). In contrast, histone H1 was degraded completely if incubated with trypsin (data not shown); degradation was prevented if trypsin activity was inhibited using PMSF. Note that the extraction buffer used for preparation of nuclear extracts contains protease inhibitors, consistent with low rates of proteolysis in debiotinylation assays. Finally, rates of histone debiotinylation were compared with rates of histone proteolysis, using nuclear extracts and trypsin as sources of enzyme.

Tissue Distribution and Cellular Localization

The activities of histone debiotinylases depended on the tissue from which cells originated. Enzyme activities in NCI-H69 lung cancer cells and Jurkat lymphoma cells were approximately twice the activities in HepG2 hepatocarcinoma cells and JAr choriocarcinoma cells (FIG. 17). Enzyme active in HC_{—}116 colon cancer cells was slightly less that the enzyme activities in NCI-H69 (FIG. 16), (P<0.05; n=3). Moreover, the activities of histone debiotinylases were greater in cell nuclei compared with cytoplasm. For example, debiotinylase activity was 1.8±0.1 pmol biotin released/(mg protein×15 min) in nuclei from NCI-H69 cells, but only 1.1±0.3 pmol biotin released/(mg protein×15 min) in cytoplasm.

Identity of Histone Debiotinylase

Biotinidase localized to the human cell nucleus, consistent with a role for biotinidase in histone debiotinylation in vivo. In immunocytochemistry experiments, the majority of anti-biotinidase antibody localized to JAr cell nuclei (data not shown). Staining with DAPI was used to confirm nuclear localization (data not shown). As a specificity control, cytoplasm was stained using rhodamine phalloidin (data not shown). The merged image is consistent with nuclear localization of biotinidase (data not shown); pre-immune serum did not generate a signal (data not shown).

Cell Cycle

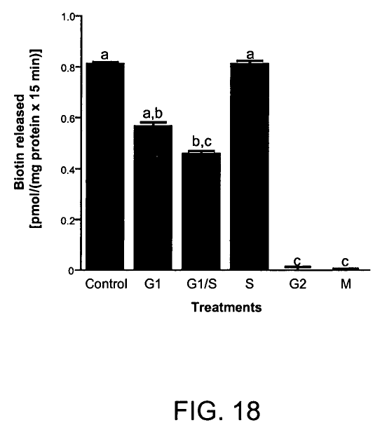

Previous studies suggested that biotinylation of histone might play a role in the regulation of cell proliferation (Stanley et al., 2001; Narang et al., 2004). Here the inventors quantified the activities of nuclear histone debiotinylases at various phases of the cell cycle. Debiotinylase activities were greater in S phase of the cell cycle compared with other phases (FIG. 18). Lowest activities were observed during G2 and M phase of the cell cycle. Note that whole cell extracts were used for analysis of M phase cells, given the disintegration of the nuclear envelope during mitosis. Potential mechanisms of histone debiotinylase regulation are reviewed in the Discussion section below.

Discussion

The inventors have developed an avidin-based assay to quantify activities of histone debiotinylases in extracts from eukaryotic cells. Using this assay, the inventors have shown (i) that human cell nuclei contain histone debiotinylase activity; (ii) that debiotinylation of histones is mediated by debiotinylases rather than proteases; (iii) that the activities of histone debiotinylases are greater in cells derived from lung and lymphoid tissues compared with liver and placenta and enzyme activity in HCT-116 colon cancer cells was slightly less that the enzyme activities in NCI-H69; (iv) that debiotinylation of histones is mediated by biotinidase and, perhaps, other histone debiotinylases; (v) that biotinidase accumulates in the cell nucleus, consistent with the cellular distribution of histone debiotinylase activity; and (vi) that the activities of histone debiotinylases depend on the cell cycle: activities are maximal during S phase, and are minimal during G2 and M phase of the cycle.

As discussed above, biotinylation of histones is believed to play a role in cell proliferation (Stanley et al., 2001; Narang et al., 2004), gene silencing (Peters et al., 2002), and the cellular response to DNA damage (Peters et al., 2002; Kothapalli and Zempleni, 2004). Deviations from the normal path in these processes are associated with detrimental events such as fetal malformations and malignant transformation. Second, enzymes that mediate the binding of biotin to histones have been well characterized (see below), but relatively little is known about the enzymes that mediate removal of the biotin mark from histones. Previous studies provided circumstantial evidence that biotinidase might mediate debiotinylation of histones (Ballard et al., 2002). The present study has demonstrated that human cell nuclei contain histone debiotinylases. Third, inborn errors causing biotinidase deficiency are fairly common in humans. The estimated incidence of profound biotinidase deficiency (<10% of normal biotinidase activity) is one in 112,271 live births, and the incidence of partial biotinidase deficiency (<30% of normal biotinidase activity) is one in 129,282 (Wolf, 1991). The combined incidence of profound and partial deficiency is 1 in 60,089 live births; an estimated 1 in 123 individuals is heterozygous for the disorder (Wolf, 1991). Mutations of the biotinidase gene have been well characterized at the molecular level (Moslinger et al., 2003; Laszlo et al., 2003; Neto et al., 2004). It remains to be determined whether biotinidase deficiency is associated with abnormal gene expression, cell proliferation, and DNA repair activity.

The present study provides evidence that histone debiotinylases play a role in cell cycle progression. The specific mechanisms regulating histone debiotinylase (biotinidase) activity during the cell cycle remain to be elucidated. Without being bound by theory, the present inventors believe that regulation could be achieved by covalent modifications of biotinidase (see below), but the identities of these modifications are currently unknown.

Biotinidase mediates the binding of biotin to histones (Hymes et al., 1995; Example 1). The present study provides evidence that biotinidase is also capable of mediating debiotinylation of histones. Without being bound by theory, the inventors believe that variables such as the microenvironment in chromatin, and posttranslational modifications, and alternate splicing of biotinidase might determine whether biotinidase acts as biotinyl histone transferase or histone debiotinylase. This theory is based on the following lines of reasoning. First, the availability of substrate might favor either biotinylation or debiotinylation of histones. For example, biocytin is a biotin donor in biotinyl transferase reactions (Hymes et al., 1995); locally high concentrations of biocytin might increase the rate of histone biotinylation in confined regions of chromatin. Second, proteins may interact with biotinidase at the chromatin level in analogy to interactions among other chromatin-remodeling enzymes (Bottomley, 2004), favoring either biotinylation or debiotinylation of histones. Third, three alternatively spliced variants of biotinidase have been identified (Stanley et al., 2004). Theoretically, these variants may have unique functions in histone metabolism. Fourth, some variants of biotinidase are modified posttranslationally by glycosylation (Stanley et al., 2004; Cole et al., 2004), potentially affecting enzymatic activity.

Enzymes other than biotinidase may also mediate debiotinylation of histones. Biotinidase belongs to the nitrilase superfamily of enzymes, which consists of 12 families of amidases, N-acyltransferases, and nitrilases Brenner, 2002). Some members of the nitrilase superfamily (vanins-1, -2, and -3) share significant sequence similarities with biotinidase (Maras et al., 1999).

Each publication cited herein is incorporated herein by reference in its entirety. In addition, all information in each sequence database accession number cited herein is incorporated by reference in entirety.

References

Zempleni, J., Biotin, in Present Knowledge in Nutrition, B. A. Bowman and R. M. Russell, Editors. 2001, ILSI Press: Washington, D.C. p. 241-252.
Solorzano-Vargas, Proc. Natl. Acad. Sci. USA, 2002. 99: p. 5325-5330.
Rodriguez-Melendez, et al. Int. J. Vitam. Nutr. Res., 2004. 74: p. 209-216.
Griffin, et al., J. Nutr., 2003. 133: p. 3409-3415.
Wiedmann, et al., J. Nutr. Biochem., 2004. 15: p. 433-439.
Rodriguez-Melendez et al., J. Nutr. Biochem., 2005. (in press).
Rodriguez-Melendez, et al., J. Nutr., 2003. 133: p. 1259-1264.
Hymes, J., et al., Biochem. Mol. Med., 1995. 56: p. 76-83.
Stanley et al., Eur. J. Biochem., 2001. 268: p. 5424-5429.
Wolffe, A., Chromatin. 3th ed. 1998, San Diego, Calif.: Academic Press.
Fischle et al., Curr. Opin. Cell Biol., 2003. 15: p. 172-183.
Jenuwein et al., Science, 2001. 293: p. 1074-1080.
Wang, et al., Science, 2004. 306(5694): p. 279-283.
Narang, et al., Hum. Mol. Genet., 2004. 13: p. 15-23.
Zempleni, Annu. Rev. Nutr., 2005. (in press).
Sarath et al., FASEB J., 2004. 18: p. A103.
Camporeale et al., Eur. J. Biochem., 2004. 271: p. 2257-2263.
Peters et al., Am. J. Physiol. Cell Physiol., 2002. 283: p. C878-C884.
Kothapalli et al., FASEB J., 2004. 18: p. A103-104.
Ballard et al., Eur. J. Nutr., 2002. 41: p. 78-84.
Manthey et al., J. Nutr., 2002. 132: p. 887-892.
Scheerger et al., Int. J. Vitam. Nutr. Res., 2003. 73: p. 461-467.
Crisp et al., Eur. J. Nutr., 2004. 43: p. 23-31.
Zempleni et al., Am. J. Physiol. Cell Physiol., 1998. 275: p. C382-C388.
Chaudhry et al., Oncogene, 2002. 21(12): p. 1934-1942.
Van Hooser et al., J. Cell Sci., 1998. 111: p. 3497-3506.
Allison et al., Cancer Res., 2003. 63(6674-6679).
Whitfield et al., Mol. Cell. Biol., 2000. 20(12): p. 4188-4198.
Vindelov, Virchows Arch. B Cell Path., 1977. 24: p. 227-242.
Cheung et al., Cell, 2003. 113: p. 507-517.
SAS Institute Inc., StatView Reference. 3th ed. 1999, Cary, N.C.: SAS Publishing.
Green, Avidin. Adv. Protein Chem., 1975. 29: p. 85-133.
Green, Avidin and Streptavidin, in Methods in Enzymology. 1990, Academic Press, Inc.: New York. p. 51-67.
Wolf, J. Inher. Metab. Dis., 1991. 14: p. 923-927.
Moslinger et al., Eur. J. Pediatr., 2003. 162 Suppl 1: p. S46-49.
Laszlo et al., J. Inherit. Metab. Dis., 2003. 26(7): p. 693-698.
Neto et al., Braz. J. Med. Biol. Res., 2004. 37(3): p. 295-299.
Bottomley, EMBO Rep., 2004. 5(5): p. 464-469.
Stanley et al., Mol. Genet. Metab., 2004. 81(4): p. 300-312.
Cole et al., J. Biol. Chem., 1994. 269(9): p. 6566-70.
Brenner, Curr. Opin. Struct. Biol., 2002. 12: p. 775-782.
Maras et al., FEBS Lett., 1999. 461: p. 149-152.
D'Amours et al., Biochem J 1999;342 (Pt 2):249-268.
Grewal et al. Science 2003;301:798-802.
Cheng et al., Annu Rev Biophys Biomol Struct 2004;
Fischle et al., Nature 2003;425:475-479.
Zhang et al., Chromosoma 2003;112:77-86.
Luger et al., Nature 1997;389:251-260.
Ausio et al. Biochem Cell Biol 2001;79:693-708.
Downs et al., Mol Cell 2004;16:979-990.
Paull et al., Curr Biol 2000;10:886-895.
Wyatt et al., Genetics 2003;164:47-64.
Pantazis et al., J Biol Chem 1981;256:4669-4675.
Goll et al., Genes Dev 2002;16:1739-1742.
Aihara et al., Genes Dev 2004;18:877-888.
Camporeale et al., Use of synthetic peptides for identifying biotinylation sites in human histones. In: McMahon, R J, McMahon, R Js. Avidin-Biotin Technology in the Life Sciences. Totowa, N.J.: Humana Press; 2005 (in press).
Garrett et al., Biochemistry Fort Worth, Tex.: Saunders College Publishing; 1995.
Turner, Cell 2002;111:285-291.
Bannister et al., Cell 2002;109:801-806.
Cuthbert et al., Cell 2004;118:545-553.
Pispa, Ann Med Exp Biol Fenniae 1965;43:4-39.
Boulikas et al., (1990), Exp. Cell Res. 187, 77-84.
Shiio et al., (2003), Proc. Natl. Acad. Sci. USA. 100, 13225-30.
Swango et al., (1998), Hum. Genet. 102, 571-575.
Wolf et al., (2002), Mol. Genet. Metab. 77, 108-111.
Yang et al., (2001), Hum. Genet. 109, 526-534.
Wolf et al. (1991) Biotinidase deficiency in Advances in Pediatrics (Barness, L. & Oski, F., eds) pp. Medical Book Publishers, Chicago, Ill.
Plath et al., (2003), Science. 300, 131-135.
Santos-Rosa et al., (2002), Nature. 419, 407-411.
Schneider et al., (2004), Nat. Cell. Biol. 6, 73-77.
Fields (1998) Solid-phase peptide synthesis in Molecular Biomethods Handbook (Rapley, R. & Walker, J. M., eds) pp. 527-545, Humana Press, Inc., Totowa, N.J.
Lachner et al., (2003), J. Cell Sci. 116, 2117-2124.
Bird, A. (2001), Science. 294, 2113-2115.
Lee et al., (1993), Cell 72, 73-84.
Durkacz et al., (1980), Nature 283, 593-596.
Althaus (1992), J. Cell Sci. 102, 663-670.
Zempleni et al., (1999), Arch. Biochem. Biophys. 371, 83-88.
Sigal et al., (1995), Mol. Immunol. 32, 623-32.
Sigal et al., (1996), Mol. Immunol. 33, 1323-33.
Smith et al., (2003), Anal. Biochem. 316, 23-33.
Ellman (1958), Arch. Biochem. Biophys. 74, 443 -450.
Hymes (1999), J. Nutr. 129, 485S-489S.
Griffin (2002), Int. J. Vitam. Nutr. Res. 72, 195-198.
Strahl et al., C. D. (2000), Nature. 403, 41-45.
Allfrey et al., (1964), Proc. Soc. Natl. Acad. Sci. USA 51, 786-794.
Mathis et al., (1978), Nucleic Acids Res. 5, 3523-3547.

While various embodiments of the present invention have been described in detail, it is apparent that modifications and adaptations of those embodiments will occur to those skilled in the art. It is to be expressly understood, however, that such modifications and adaptations are within the scope of the present invention, as set forth in the following claims.

What is claimed is:

1. An isolated antibody or antigen-binding fragment thereof that selectively binds to a biotinylated histone selected from the group consisting of biotinylated histone H2A, biotinylated histone H3, and biotinylated histone H4.

2. The isolated antibody or antigen-binding fragment thereof of claim 1, wherein the antibody or antigen-binding fragment thereof does not bind to a non-biotinylated histone.

3. The isolated antibody or antigen-binding fragment thereof of claim 1, wherein the antibody or antigen binding fragment thereof selectively binds to biotinylated histone H4.

4. The isolated antibody or antigen-binding fragment thereof of claim 3, wherein the antibody or antigen binding fragment thereof selectively binds to:
a) an epitope comprising the second lysine residue from the N-terminus in histone H4, wherein the second lysine residue is biotinylated; or
b) an epitope comprising the third lysine residue from the N-terminus in histone H4, wherein the third lysine residue is biotinylated.

5. The isolated antibody or antigen-binding fragment thereof of claim 3, wherein the antibody or antigen binding fragment thereof selectively binds to:
a) an epitope comprising the lysine at position 8 of SEQ ID NO:6, or the equivalent position thereto in a non-human histone H4 sequence, wherein the lysine residue is biotinylated; or
b) an epitope comprising the lysine at position 12 of SEQ ID NO:6, or the equivalent position thereto in a non-human histone H4 sequence, wherein the lysine residue is biotinylated.

6. The isolated antibody or antigen-binding fragment thereof of claim 3, wherein the antibody or antigen binding fragment thereof selectively binds to an amino acid sequence selected from the group consisting of: SEQ ID NO:6, SEQ ID NO:7 and SEQ ID NO:10, wherein said amino acid sequence is biotinylated.

7. The isolated antibody or antigen-binding fragment thereof of claim 3, wherein the antibody or antigen binding fragment thereof does not cross-react with histones H1, H2A, H2B and H3.

8. The isolated antibody or antigen-binding fragment thereof of claim 1, wherein the antibody or antigen binding fragment thereof selectively binds to biotinylated histone H3.

9. The isolated antibody or antigen-binding fragment thereof of claim 8, wherein the antibody or antigen binding fragment thereof selectively binds to:
a) an epitope comprising the first lysine residue from the N-terminus in histone H3, wherein the first lysine residue is biotinylated;
b) an epitope comprising the second lysine residue from the N-terminus in histone H3, wherein the second lysine residue is biotinylated; or
c) an epitope comprising the fourth lysine residue from the N-terminus in histone H3, wherein the fourth lysine residue is biotinylated.

10. The isolated antibody or antigen-binding fragment thereof of claim 8, wherein the antibody or antigen binding fragment thereof selectively binds to:
a) an epitope comprising the lysine at position 4 of SEQ ID NO:5, or the equivalent position thereto in a non-human histone H3 sequence, wherein the lysine residue is biotinylated;
b) an epitope comprising the lysine at position 9 of SEQ ID NO:5, or the equivalent position thereto in a non-human histone H3 sequence, wherein the lysine residue is biotinylated; or
c) an epitope comprising the lysine at position 18 of SEQ ID NO:5, or the equivalent position thereto in a non-human histone H3 sequence, wherein the lysine residue is biotinylated.

11. The isolated antibody or antigen-binding fragment thereof of claim 8, wherein the antibody or antigen binding fragment thereof selectively binds to an amino acid sequence selected from the group consisting of: SEQ ID NO:5, SEQ ID NO:30 and SEQ ID NO:32, wherein said amino acid sequence is biotinylated.

12. The isolated antibody or antigen-binding fragment thereof of claim 8, wherein the antibody or antigen binding fragment thereof does not cross-react with histones H1, H2A, H2B and H4.

13. The isolated antibody or antigen-binding fragment thereof of claim 1, wherein the antibody or antigen binding fragment thereof selectively binds to biotinylated histone H2A.

14. The isolated antibody or antigen-binding fragment thereof of claim 13, wherein the antibody or antigen binding fragment thereof selectively binds to:
a) an epitope comprising the second lysine residue from the N-terminus in histone H2A, wherein the second lysine residue is biotinylated;
b) an epitope comprising the third lysine residue from the N-terminus in histone H2A, wherein the third lysine residue is biotinylated;
c) an epitope comprising the first lysine residue from the C-terminus in histone H2A, wherein the first lysine residue is biotinylated;
d) an epitope comprising the second lysine residue from the C-terminus in histone H2A, wherein the second lysine residue is biotinylated; or
e) an epitope comprising the third lysine residue from the C-terminus in histone H2A, wherein the third lysine residue is biotinylated.

15. The isolated antibody or antigen-binding fragment thereof of claim 13, wherein the antibody or antigen binding fragment thereof selectively binds to:
a) an epitope comprising the lysine at position 9 of SEQ ID NO:2, or the equivalent position thereto in a non-human histone H2A sequence, wherein the lysine residue is biotinylated;
b) an epitope comprising the lysine at position 13 of SEQ ID NO:2, or the equivalent position thereto in a non-human histone H2A sequence, wherein the lysine residue is biotinylated;
c) an epitope comprising the lysine at position 125 of SEQ ID NO:2, or the equivalent position thereto in a non-human histone H2A sequence, wherein the lysine residue is biotinylated;
d) an epitope comprising the lysine at position 127 of SEQ ID NO:2, or the equivalent position thereto in a non-human histone H2A sequence, wherein the lysine residue is biotinylated; or
e) an epitope comprising the lysine at position 129 of SEQ ID NO:2, or the equivalent position thereto in a non-human histone H2A sequence, wherein the lysine residue is biotinylated.

16. The isolated antibody or antigen-binding fragment thereof of claim 13, wherein the antibody or antigen binding fragment thereof selectively binds to an amino acid sequence selected from the group consisting of: SEQ ID NO:2, SEQ ID NO:3, SEQ ID NO:48, SEQ ID NO:49 and SEQ ID NO:52, wherein said amino acid sequence is biotinylated.

17. The isolated antibody or antigen-binding fragment thereof of claim 13, wherein the antibody or antigen binding fragment thereof does not cross-react with histones H1, H2B, H3, and H4.

18. The isolated antibody or antigen-binding fragment thereof of claim 1, wherein the antibody is a monoclonal antibody.

19. The isolated antibody or antigen-binding fragment thereof of claim 1, wherein the antigen binding fragment is an Fab fragment.

20. The isolated antibody or antigen-binding fragment thereof of claim 1, wherein the antibody is a humanized antibody.

21. The isolated antibody or antigen-binding fragment thereof of claim 1, wherein the antibody is a bispecific antibody.

22. The isolated antibody or antigen-binding fragment thereof of claim 1, wherein the antibody is a monovalent antibody.

23. A composition comprising the isolated antibody or antigen binding fragment thereof of claim 1.

24. A delivery vehicle comprising the isolated antibody or antigen binding fragment thereof of claim 1 linked to an agent to be delivered.

25. A method to detect biotinylated histones in a biological sample, comprising: contacting a biological sample containing histones with an antibody or antigen-binding fragment thereof of claim 1, and detecting the amount of antibody or antigen-binding fragment thereof that binds to the biological sample.

26. The method of claim 25, wherein the biological sample is a eukaryotic cell sample or a nuclear extract thereof.

27. A method to detect DNA damage in a cell, comprising contacting a nuclear extract from a cell or tissue to be evaluated with an antibody or antigen-binding fragment thereof according to claim 1, and measuring the amount of antibody that binds to histones in the extract as compared to a control sample that does not have DNA damage.

28. A method to detect biotinyl transferase activity in a biological sample, comprising:
a) contacting a biological sample with a histone or polypeptide fragment thereof, wherein the polypeptide fragment thereof comprises at least one biotinylation site in the histone, and wherein the histone or polypeptide fragment thereof is not biotinylated prior to contact with the biological sample;
b) incubating the biological sample and histone or polypeptide fragment thereof with biocytin or biotin and ATP; and
c) measuring the amount of histone or polypeptide fragment thereof that is biotinylated after step (b), wherein the amount of biotinylated histone or polypeptide fragment thereof is indicative of the amount of biotinyl transferase activity in the biological sample.

29. The method of claim 28, wherein the biological sample is a nuclear extract from a mammalian cell.

30. The method of claim 28, wherein the histone is selected from the group consisting of histone H1, histone H2A, histone H2B, histone H3 and histone H4.

31. The method of claim 28, wherein the polypeptide fragment thereof is an at least about 8 amino acid polypeptide fragment selected from the group consisting of:
a) a polypeptide fragment of human histone H4 (SEQ ID NO:6), comprising at least one lysine residue selected from the group consisting of: the lysine at position 8 and the lysine at position 12;
b) a polypeptide fragment of human histone H3 (SEQ ID NO:5), comprising at least one lysine residue selected from the group consisting of: the lysine at position 4, the lysine at position 9 and the lysine at position 18;
c) a polypeptide fragment of human histone H2A (SEQ ID NO:2) or H2A.X (SEQ ID NO:3), comprising at least one lysine residue selected from the group consisting of: the lysine at position 9 and the lysine at position 13; and
d) a polypeptide fragment of human histone H2A (SEQ ID NO:2), comprising at least one lysine residue selected from the group consisting of: the lysine at position 125, the lysine at position 127 and the lysine at position 129.

32. The method of claim 28, wherein step (c) comprises detecting the amount of biotinylated histones or polypeptide fragments thereof by contacting the histones or polypeptide fragments thereof with an antibody that selectively binds to the histone or polypeptide fragment when the histone or polypeptide fragment is biotinylated and not to non-biotinylated histone or polypeptide fragment thereof.

33. The method of claim 28, wherein the histone or polypeptide fragment in step (a) are immobilized in an assay well, and wherein step (c) comprises the steps of:
i) washing the assay well to remove the biological sample and biocytin;
ii) incubating the immobilized histone or polypeptide fragment with an antibody that selectively binds to the histone or polypeptide fragment when the histone or polypeptide fragment is biotinylated and not to non-biotinylated histone or polypeptide fragment thereof; and
iii) measuring the amount of antibody in (ii) that is bound to the biotinylated histone or polypeptide fragment thereof to indicate the amount of biotinyl transferase activity in the biological sample.

34. The method of claim 33, wherein step (iii) comprises contacting the antibody with a labeled secondary antibody and detecting the amount of bound label.

35. The method of claim 28, wherein step (c) comprises the steps of:
i) separating the proteins and polypeptides after step (b) by gel electrophoresis;
ii) performing an immunoblot of the gel using an antibody that selectively binds to the histone or polypeptide fragment when the histone or polypeptide fragment is biotinylated and not to non-biotinylated histone or polypeptide fragment thereof; and
iii) measuring the amount of antibody in (ii) that is bound to the biotinylated histone or polypeptide fragment thereof to indicate the amount of biotinyl transferase activity in the biological sample.

36. An assay to detect debiotinylase activity in a biological sample, comprising:
a) incubating a biological sample with a biotinylated histone or a biotinylated polypeptide fragment thereof;
b) contacting the biological sample and biotinylated histone or fragment thereof with an avidin-conjugated detectable label; and
c) measuring the amount of avidin-conjugated detectable label that is bound to the biotinylated histone or fragment thereof after incubation with the biological sample as compared to prior to the incubation step, wherein an amount of reduction in the biotinylation of the histone or fragment thereof after the incubation step indicates the amount of debiotinylase activity in the biological sample.

37. A method to identify regulators of histone biotinylation, comprising:
a) contacting a putative regulatory compound of histone biotinylation with a histone or a polypeptide fragment thereof, wherein the polypeptide fragment thereof comprises at least one biotinylation site in the histone, and wherein the histone or polypeptide fragment thereof is not biotinylated prior to contact with the biological sample;
b) contacting the histone or polypeptide fragment thereof with an enzyme selected from the group consisting of biotinidase and holocarboxylase synthetase, either after step (a) or at the same time as step (a);
c) contacting the histone or polypeptide fragment thereof with a substrate for the enzyme in (b), either after step (b) or at the same time as step (b); and
d) measuring the amount of histone or polypeptide fragment thereof that is biotinylated after step (c), wherein a decrease in the amount of biotinylated histone or polypeptide fragment thereof in the presence of the putative regulatory compound as compared to in the absence of the putative regulatory compound indicates that the putative regulatory compound is an inhibitor of histone biotinylation, and wherein an increase in the amount of biotinylated histone or polypeptide fragment thereof in the presence of the putative regulatory compound as compared to in the absence of the putative regulatory compound indicates that the putative regulatory compound is an enhancer of histone biotinylation.

38. The method of claim 37, wherein step (c) comprises detecting the amount of biotinylated histones or polypeptide fragments thereof by contacting the histones or polypeptide fragments thereof with an antibody that selectively binds to the histone or polypeptide fragment when the histone or polypeptide fragment is biotinylated and not to non-biotinylated histone or polypeptide fragment thereof.

39. The method of claim 37, wherein the histone is selected from the group consisting of histone H1, histone H2A, histone H2B, histone H3 and histone H4.

40. The method of claim 37, wherein the polypeptide fragment thereof is an at least about 8 amino acid polypeptide fragment selected from the group consisting of:
a) a polypeptide fragment of human histone H4 (SEQ ID NO:6), comprising at least one lysine residue selected from the group consisting of: the lysine at position 8 and the lysine at position 12;
b) a polypeptide fragment of human histone H3 (SEQ ID NO:5), comprising at least one lysine residue selected from the group consisting of: the lysine at position 4, the lysine at position 9 and the lysine at position 18;
c) a polypeptide fragment of human histone H2A (SEQ ID NO:2) or H2A.X (SEQ ID NO:3), comprising at least one lysine residue selected from the group consisting of: the lysine at position 9 and the lysine at position 13; and
d) a polypeptide fragment of human histone H2A (SEQ ID NO:2), comprising at least one lysine residue selected from the group consisting of: the lysine at position 125, the lysine at position 127 and the lysine at position 129.


SEQUENCE LISTING (91 sequences)

SEQ ID NO: 1 - protein, 215 aa, Homo sapiens
MSETVPPAPAASAAPEKPLAGKKAKKPAKAAAASKKKPAGPSVSELIVQAASSSKERGGVSLAALKKALAAAGYDVEKNNSRIKLGIKSLVSKGTLVQTKGTGASGSFKLNKKASSVETKPGASKVATKTKATGASKKLKKATGASKKSVKTPKKAKKPAATRKSSKNPKKPKTVKPKKVAKSPAKAKAVKPKAAKARVTKPKTAKPKKAAPKKK

SEQ ID NO: 2 - protein, 130 aa, Homo sapiens
MSGRGKQGGKARAKAKTRSSRAGLQFPVGRVHRLLRKGNYSERVGAGAPVYLAAVLEYLTAEILELAGNAARDNKKTRIIPRHLQLAIRNDEELNKLLGRVTIAQGGVLPNIQAVLLPKKTESHHKAKGK

SEQ ID NO: 3 - protein, 143 aa, Homo sapiens
MSGRGKTGGKARAKAKSRSSRAGLQFPVGRVHRLLRKGHYAERVGAGAPVYLAAVLEYLTAEILELAGNAARDNKKTRIIPRHLQLAIRNDEELNKLLGGVTIAQGGVLPNIQAVLLPKKTSATVGPKAPSGGKKATQASQEY

SEQ ID NO: 4 - protein, 126 aa, Homo sapiens
MPEPAKSAPAPKKGSKKAVTKAQKKDGKKRKRSRKESYSVYVYKVLKQVHPDTGISSKAMGIMNSFVNDIFERIAGEASRLAHYNKRSTITSREIQTAVRLLLPGELAKHAVSEGTKAVTKYTSSK

SEQ ID NO: 5 - protein, 136 aa, Homo sapiens [sequence omitted]

SEQ ID NO: 6 - protein, 103 aa, Homo sapiens [sequence omitted]

SEQ ID NO: 7 - protein, 19 aa, Artificial
  Chemically Synthesized
SGRGKGGKGLGKGGAKRHR

SEQ ID NO: 8 - protein, 21 aa, Artificial
  Chemically Synthesized
TAMDVVYALKRQGRTLYGFGG

SEQ ID NO: 9 - protein, 7 aa, Artificial
  Chemically Synthesized
GGABBRC

SEQ ID NO: 10 - protein, 10 aa, Artificial
  Chemically Synthesized
  MOD_RES (1)..(1): acetyl-alpha-NH2-L-glycine
GGKGLGKGGA

SEQ ID NO: 11 - protein, 10 aa, Artificial
  Chemically Synthesized
  MOD_RES (1)..(1): acetyl-alpha-NH2-L-glycine
GGAGLGKGGA

SEQ ID NO: 12 - protein, 10 aa, Artificial
  Chemically Synthesized
  MOD_RES (1)..(1): acetyl-alpha-NH2-L-glycine
GGKGLGAGGA

SEQ ID NO: 13 - protein, 10 aa, Artificial
  Chemically synthesized
  MOD_RES (1)..(1): acetyl-alpha-NH2-L-glycine
GGAGLGAGGA

SEQ ID NO: 14 - protein, 10 aa, Artificial
  Chemically Synthesized
  MOD_RES (1)..(1): acetyl-alpha-NH2-L-glycine
  MOD_RES (3)..(3): acetyl-epsilon-NH2-L-lysine
GGKGLGKGGA

SEQ ID NO: 15 - protein, 10 aa, Artificial
  Chemically Synthesized
  MOD_RES (1)..(1): acetyl-alpha-NH2-L-glycine
  MOD_RES (7)..(7): acetyl-epsilon-NH2-L-lysine
GGKGLGKGGA

SEQ ID NO: 16 - protein, 10 aa, Artificial
  Chemically Synthesized
  MOD_RES (1)..(1): acetyl-alpha-NH2-L-glycine
  MOD_RES (3)..(3): dimethyl-epsilon-NH2-L-lysine
GGKGLGKGGA

SEQ ID NO: 17 - protein, 10 aa, Artificial
  Chemically Synthesized
  MOD_RES (1)..(1): acetyl-alpha-NH2-L-glycine
  MOD_RES (7)..(7): dimethyl-epsilon-NH2-L-lysine
GGKGLGKGGA

SEQ ID NO: 18 - protein, 10 aa, Artificial
  Chemically Synthesized
  MOD_RES (1)..(1): acetyl-alpha-NH2-L-glycine
  MOD_RES (7)..(7): formyl-epsilon-NH2-L-lysine
GGKGLGKGGA

SEQ ID NO: 19 - protein, 10 aa, Artificial
  Chemically Synthesized
  MOD_RES (1)..(1): acetyl-alpha-NH2-L-glycine
  MOD_RES (3)..(3): biotin-epsilon-NH2-L-lysine
GGKGLGKGGA

SEQ ID NO: 20 - protein, 10 aa, Artificial
  Chemically Synthesized
  MOD_RES (1)..(1): acetyl-alpha-NH2-L-glycine
  MOD_RES (7)..(7): biotin-epsilon-NH2-L-lysine
GGKGLGKGGA

SEQ ID NO: 21 - protein, 10 aa, Artificial
  Chemically Synthesized
  MOD_RES (1)..(1): acetyl-alpha-NH2-L-glycine
GGRGLGKGGA

SEQ ID NO: 22 - protein, 10 aa, Artificial
  Chemically Synthesized
  MOD_RES (1)..(1): acetyl-alpha-NH2-L-glycine
GGKGLGRGGA

SEQ ID NO: 23 - protein, 10 aa, Artificial
  Chemically Synthesized
  MOD_RES (1)..(1): acetyl-alpha-NH2-L-glycine
GGRGLGRGGA

SEQ ID NO: 24 - protein, 10 aa, Artificial
  CHEMICALLY SYNTHESIZED
  MOD_RES (1)..(1): acetyl-alpha-NH2-L-glycine
GGEGLGKGGA

SEQ ID NO: 25 - protein, 10 aa, Artificial
  CHEMICALLY SYNTHESIZED
  MOD_RES (1)..(1): acetyl-alpha-NH2-L-glycine
GGKGLGEGGA

SEQ ID NO: 26 - protein, 10 aa, Artificial
  CHEMICALLY SYNTHESIZED
  MOD_RES (1)..(1): acetyl-alpha-NH2-L-glycine
GGQGLGKGGA

SEQ ID NO: 27 - protein, 10 aa, Artificial
  CHEMICALLY SYNTHESIZED
  MOD_RES (1)..(1): acetyl-alpha-NH2-L-glycine
GGKGLGQGGA

SEQ ID NO: 28 - protein, 10 aa, Artificial
  CHEMICALLY SYNTHESIZED
  MOD_RES (1)..(1): acetyl-alpha-NH2-L-glycine
GGQGLGQGGA

SEQ ID NO: 29 - protein, 10 aa, Artificial
  CHEMICALLY SYNTHESIZED
  MOD_RES (1)..(1): acetyl-alpha-NH2-L-glycine
  MOD_RES (7)..(7): D-lysine
GGKGLGKGGA

SEQ ID NO: 30 - protein, 25 aa, Artificial
  CHEMICALLY SYNTHESIZED
ARTKQTARKSTGGKAPRKQLATKAA

SEQ ID NO: 31 - protein, 25 aa, Artificial
  CHEMICALLY SYNTHESIZED
APRKQLATKAARKSAPATGGVKKPH

SEQ ID NO: 32 - protein, 21 aa, Artificial
  CHEMICALLY SYNTHESIZED
KRVTIMPKDIQLARRIRGERA

SEQ ID NO: 33 - protein, 14 aa, Artificial
  CHEMICALLY SYNTHESIZED
  MOD_RES (4)..(4): BIOTINYLATION
ARTKQTARKSTGGC

SEQ ID NO: 34 - protein, 14 aa, Artificial
  CHEMICALLY SYNTHESIZED
  MOD_RES (9)..(9): BIOTINYLATION
ARTKQTARKSTGGC

SEQ ID NO: 35 - protein, 14 aa, Artificial
  CHEMICALLY SYNTHESIZED
  MOD_RES (6)..(6): BIOTINYLATION
GKAPRKQLATKAAC

SEQ ID NO: 36 - protein, 8 aa, Artificial
  CHEMICALLY SYNTHESIZED
TARKSTGG

SEQ ID NO: 37 - protein, 8 aa, Artificial
  CHEMICALLY SYNTHESIZED
TAAKSTGG

SEQ ID NO: 38 - protein, 8 aa, Artificial
  CHEMICALLY SYNTHESIZED
  MOD_RES (3)..(3): Orn
TAXKSTGG

SEQ ID NO: 39 - protein, 8 aa, Artificial
  CHEMICALLY SYNTHESIZED
  MOD_RES (5)..(5): PHOSPHORYLATION
TARKSTGG

SEQ ID NO: 40 - protein, 8 aa, Artificial
  CHEMICALLY SYNTHESIZED
TARASTGG

SEQ ID NO: 41 - protein, 8 aa, Artificial
  CHEMICALLY SYNTHESIZED
TRAKSTGG

SEQ ID NO: 42 - protein, 13 aa, Artificial
  CHEMICALLY SYNTHESIZED
  MOD_RES (9)..(9): BIOTINYLATION
SGRGKQGGKARAC

SEQ ID NO: 43 - protein, 16 aa, Artificial
  CHEMICALLY SYNTHESIZED
  MOD_RES (4)..(4): BIOTINYLATION
ARAKAKTRSSRAGLQC

SEQ ID NO: 44 - protein, 543 aa, Homo sapiens
MAHAHIQGGRRAKSRFVVCIMSGARSKLALFLCGCYVVALGAHTGEESVADHHEAEYYVAAVYEHPSILSLNPLALISRQEALELMNQNLDIYEQQVMTAAQKDVQIIVFPEDGIHGFNFTRTSIYPFLDFMPSPQVVRWNPCLEPHRFNDTEVLQRLSCMAIRGDMFLVANLGTKEPCHSSDPRCPKDGRYQFNTNVVFSNNGTLVDRYRKHNLYFEAAFDVPLKVDLITFDTPFAGRFGIFTCFDILFFDPAIRVLRDYKVKHVVYPTAWMNQLPLLAAIEIQKAFAVAFGINVLAANVHHPVLGMTGSGIHTPLESFWYHDMENPKSHLIIAQVAKNPVGLIGAENATGETDPSHSKFLKILSGDPYCEKDAQEVHCDEATKWNVNAPPTFHSEMMYDNFTLVPVWGKEGYLHVCSNGLCCYLLYERPTLSKELYALGVFDGLHTVHGTYYIQVCALVRCGGLGFDTCGQEITEATGIFEFHLWGNFSTSYIFPLFLTSGMTLEVPDQLGWENDHYFLRKSRLSSGLVTAALYGRLYERD

SEQ ID NO: 45 - protein, 24 aa, Artificial
  CHEMICALLY SYNTHESIZED
CLRKSRLSSGLVTAALYGRLYERD

SEQ ID NO: 46 - protein, 726 aa, Homo sapiens
MEDRLHMDNGLVPQKIVSVHLQDSTLKEVKDQVSNKQAQILEPKPEPSLEIKPEQDGMEHVGRDDPKALGEEPKQRRGSASGSEPAGDSDRGGGPVEHYHLHLSSCHECLELENSTIESVKFASAENIPDLPYDYSSSLESVADETSPEREGRRVNLTGKAPNILLYVGSDSQEALGRFHEVRSVLADCVDIDSYILYHLLEDSALRDPWTDNCLLLVIATRESIPEDLYQKFMAYLSQGGKVLGLSSSFTFGGFQVTSKGALHKTVQNLVFSKADQSEVKLSVLSSGCRYQEGPVRLSPGRLQGHLENEDKDRMIVHVPFGTRGGEAVLCQVHLELPPSSNIVQTPEDFNLLKSSNFRRYEVLREILTTLGLSCDMKQVPALTPLYLLSAAEEIRDPLMQWLGKHVDSEGEIKSGQLSLRFVSSYVSEVEITPSCIPVVTNMEAFSSEHFNLEIYRQNLQTKQLGKVILFAEVTPTTMRLLDGLMFQTPQEMGLIVIAARQTEGKGRGGNVWLSPVGCALSTLLISIPLRSQLGQRIPFVQHLMSVAVVEAVRSIPEYQDINLRVKWPNDIYYSDLMKIGGVLVNSTLMGETFYILIGCGFNVTNSNPTICINDLITEYNKQHKAELKPLRADYLIARVVTVLEKLIKEFQDKGPNSVLPLYYRYWVHSGQQVHLGSAEGPKVSIVGLDDSGFLQVHQEGGEVVTVHPDGNSFDMLRNLILPKRR

SEQ ID NO: 47 - protein, 21 aa, Artificial
  CHEMICALLY SYNTHESIZED
EHVGRDDPKALGEEPKQRRGC

SEQ ID NO: 48 - protein, 9 aa, Artificial
  CHEMICALLY SYNTHESIZED
SGRGKQGGK

SEQ ID NO: 49 - protein, 8 aa, Artificial
  CHEMICALLY SYNTHESIZED
GGKARAKA

SEQ ID NO: 50 - protein, 9 aa, Artificial
  CHEMICALLY SYNTHESIZED
AKAKTRSSR

SEQ ID NO: 51 - protein, 9 aa, Artificial
  CHEMICALLY SYNTHESIZED
AVLLPKKTE

SEQ ID NO: 52 - protein, 8 aa, Artificial
  CHEMICALLY SYNTHESIZED
SHHKAKGK

SEQ ID NO: 53 - protein, 9 aa, Artificial
  CHEMICALLY SYNTHESIZED
SGRGKQGGA

SEQ ID NO: 54 - protein, 9 aa, Artificial
  CHEMICALLY SYNTHESIZED
SGRGAQGGK

SEQ ID NO: 55 - protein, 9 aa, Artificial
  CHEMICALLY SYNTHESIZED
SGRGAQGGA

SEQ ID NO: 56 - protein, 9 aa, Artificial
  CHEMICALLY SYNTHESIZED
AKAATRSSR

SEQ ID NO: 57 - protein, 9 aa, Artificial
  CHEMICALLY SYNTHESIZED
AAAKTRSSR

SEQ ID NO: 58 - protein, 9 aa, Artificial
  CHEMICALLY SYNTHESIZED
AAAATRSSR

SEQ ID NO: 59 - protein, 9 aa, Artificial
  CHEMICALLY SYNTHESIZED
SGRGKTGGK

SEQ ID NO: 60 - protein, 9 aa, Artificial
  CHEMICALLY SYNTHESIZED
AKAKSRSSR

SEQ ID NO: 61 - protein, 9 aa, Artificial
  CHEMICALLY SYNTHESIZED
SGRGKTGGA

SEQ ID NO: 62 - protein, 9 aa, Artificial
  CHEMICALLY SYNTHESIZED
SGRGATGGK

SEQ ID NO: 63 - protein, 9 aa, Artificial
  CHEMICALLY SYNTHESIZED
AKAASRSSR

SEQ ID NO: 64 - protein, 9 aa, Artificial
  CHEMICALLY SYNTHESIZED
AAAKSRSSR

SEQ ID NO: 65 - protein, 9 aa, Artificial
  CHEMICALLY SYNTHESIZED
AAAASRSSR

SEQ ID NO: 66 - protein, 8 aa, Artificial
  CHEMICALLY SYNTHESIZED
SHHAAAGK

SEQ ID NO: 67 - protein, 8 aa, Artificial
  CHEMICALLY SYNTHESIZED
SHHAAKGA

SEQ ID NO: 68 - protein, 8 aa, Artificial
  CHEMICALLY SYNTHESIZED
SHHAAAGA

SEQ ID NO: 69 - protein, 8 aa, Artificial
  CHEMICALLY SYNTHESIZED
SHHAAAGA

SEQ ID NO: 70 - protein, 9 aa, Artificial
  CHEMICALLY SYNTHESIZED
AVLLPKKTS

SEQ ID NO: 71 - protein, 10 aa, Artificial
  CHEMICALLY SYNTHESIZED
ATVGPKAPSG

SEQ ID NO: 72 - protein, 11 aa, Artificial
  CHEMICALLY SYNTHESIZED
GKKATQASQEY

SEQ ID NO: 73 - protein, 9 aa, Artificial
  CHEMICALLY SYNTHESIZED
  MOD_RES (5)..(5): ACETYLATION
SGRGKTGGK

SEQ ID NO: 74 - protein, 9 aa, Artificial
  CHEMICALLY SYNTHESIZED
  MOD_RES (9)..(9): ACETYLATION
SGRGKTGGK

SEQ ID NO: 75 - protein, 8 aa, Artificial
  CHEMICALLY SYNTHESIZED
  MOD_RES (7)..(7): DIMETHYLATION
GGKARAKA

SEQ ID NO: 76 - protein, 8 aa, Artificial
  CHEMICALLY SYNTHESIZED
  MOD_RES (7)..(7): ACETYLATION
GGKARAKA

SEQ ID NO: 77 - protein, 10 aa, Artificial
  CHEMICALLY SYNTHESIZED
  MOD_RES (3)..(3): BIOTINYLATION
GGKGLGKGGA

SEQ ID NO: 78 - protein, 10 aa, Artificial
  CHEMICALLY SYNTHESIZED
  MOD_RES (7)..(7): BIOTINYLATION
GGKGLGKGGA

SEQ ID NO: 79 - protein, 23 aa, Artificial
  CHEMICALLY SYNTHESIZED
LRKSRLSSGLVTAALYGRLYERD

SEQ ID NO: 80 - protein, 9 aa, Artificial
  CHEMICALLY SYNTHESIZED
ARTAQTARA

SEQ ID NO: 81 - protein, 9 aa, Artificial
  CHEMICALLY SYNTHESIZED
ARTAQTARK

SEQ ID NO: 82 - protein, 9 aa, Artificial
  CHEMICALLY SYNTHESIZED
ARTKQTARA

SEQ ID NO: 83 - protein, 8 aa, Artificial
  CHEMICALLY SYNTHESIZED
ASTGGAAP

SEQ ID NO: 84 - protein, 8 aa, Artificial
  CHEMICALLY SYNTHESIZED
KSTGGAAP

SEQ ID NO: 85 - protein, 8 aa, Artificial
  CHEMICALLY SYNTHESIZED
ASTGGKAP

SEQ ID NO: 86 - protein, 8 aa, Artificial
  CHEMICALLY SYNTHESIZED
PRAQLATA

SEQ ID NO: 87 - protein, 8 aa, Artificial
  CHEMICALLY SYNTHESIZED
PRKQLATA

SEQ ID NO: 88 - protein, 8 aa, Artificial
  CHEMICALLY SYNTHESIZED
PRAQLATK

SEQ ID NO: 89 - protein, 8 aa, Artificial
  CHEMICALLY SYNTHESIZED
AQLATAAA

SEQ ID NO: 90 - protein, 8 aa, Artificial
  CHEMICALLY SYNTHESIZED
KQLATAAA

SEQ ID NO: 91 - protein, 8 aa, Artificial
  CHEMICALLY SYNTHESIZED
AQLATKAA